SEQUENCE LISTING (198 sequences)

SEQ ID NO: 1 - protein, 120 aa, Artificial Sequence
  VH TPP-8236
EVQLLESGGGLVQPGGSLRLSCAASGFTFSDYYMNWVRQAPGKGLEWVSTISGTGAAAFYADSVRGRFTISRDNSKNTLYLQMNSLRAEDTAVYYCARDQYLTGGAFDIWGQGTLVTVSS

SEQ ID NO: 2 - protein, 5 aa, Artificial Sequence
  HCDR1 TPP-8236
DYYMN

SEQ ID NO: 3 - protein, 17 aa, Artificial Sequence
  HCDR2 TPP-8236
TISGTGAAAFYADSVRG

SEQ ID NO: 4 - protein, 11 aa, Artificial Sequence
  HCDR3 TPP-8236
DQYLTGGAFDI

SEQ ID NO: 5 - protein, 110 aa, Artificial Sequence
  VL TPP-8236
QSVLTQPPSASGTPGQRVTISCSGSSSNIGNNAVNWYQQLPGTAPKLLIYGNSNRPSGVPDRFSGSKSGTSASLAISGLRSEDEADYYCSSYRGSSTLVVFGGGTKLTVL

SEQ ID NO: 6 - protein, 13 aa, Artificial Sequence
  LCDR1 TPP-8236
SGSSSNIGNNAVN

SEQ ID NO: 7 - protein, 7 aa, Artificial Sequence
  LCDR2 TPP-8236
GNSNRPS

SEQ ID NO: 8 - protein, 11 aa, Artificial Sequence
  LCDR3 TPP-8236
SSYRGSSTLVV

SEQ ID NO: 9 - DNA, 360 bp, Artificial Sequence
  VH TPP-8236
gaagtgcagctgctggaaagcggcggaggcctggtgcagcctggcggatctctgagactgagctgtgccgccagcggcttcaccttcagcgactactacatgaactgggtgcgccaggcccctggcaagggactggaatgggtgtccaccatcagcggcacaggcgccgctgccttttacgccgattctgtgcggggcagattcaccatctcccgggacaacagcaagaacaccctgtacctgcagatgaacagcctgcgggccgaggacaccgccgtgtactactgtgccagggaccagtacctgaccggcggagcctttgacatctggggccagggaacactcgtgaccgtgtcctca

SEQ ID NO: 10 - DNA, 330 bp, Artificial Sequence
  VL TPP-8236
cagtctgtgctgacacagcctcctagcgcctctggcacacctggccagagagtgaccatcagctgtagcggcagcagctccaacatcggcaacaacgccgtgaactggtatcagcagctgcccggcacagcccccaaactgctgatctacggcaacagcaaccggcccagcggcgtgcccgatagattttccggcagcaagagcggcaccagcgccagcctggctatcagcggactgagaagcgaggacgaggccgactactactgcagcagctacagaggcagcagcaccctggtggtgtttggcggaggcaccaagctgaccgtgcta

SEQ ID NO: 11 - protein, 449 aa, Artificial Sequence
  Heavy Chain TPP-8236
EVQLLESGGGLVQPGGSLRLSCAASGFTFSDYYMNWVRQAPGKGLEWVSTISGTGAAAFYADSVRGRFTISRDNSKNTLYLQMNSLRAEDTAVYYCARDQYLTGGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 216 aa, Artificial Sequence
  Light Chain TPP-8236
QSVLTQPPSASGTPGQRVTISCSGSSSNIGNNAVNWYQQLPGTAPKLLIYGNSNRPSGVPDRFSGSKSGTSASLAISGLRSEDEADYYCSSYRGSSTLVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 13 - DNA, 1347 bp, Artificial Sequence
  Heavy Chain TPP-8236
gaagtgcagctgctggaaagcggcggaggcctggtgcagcctggcggatctctgagactgagctgtgccgccagcggcttcaccttcagcgactactacatgaactgggtgcgccaggcccctggcaagggactggaatgggtgtccaccatcagcggcacaggcgccgctgccttttacgccgattctgtgcggggcagattcaccatctcccgggacaacagcaagaacaccctgtacctgcagatgaacagcctgcgggccgaggacaccgccgtgtactactgtgccagggaccagtacctgaccggcggagcctttgacatctggggccagggaacactcgtgaccgtgtcctcagccagcaccaagggccccagcgtgttccctctggcccctagcagcaagagcacatctggcggaacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagctctctgggcacccagacctacatctgcaacgtgaaccacaagcccagcaacaccaaggtggacaagaaggtggaacccaagagctgcgacaagacccacacctgtcccccttgtcctgcccccgaactgctgggaggcccttccgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagcctagagaggaacagtacaacagcacctaccgggtggtgtccgtgctgacagtgctgcaccaggactggctgaacggcaaagagtacaagtgcaaggtgtccaacaaggccctgcctgcccccatcgagaaaaccatcagcaaggccaagggccagccccgcgaaccccaggtgtacacactgcccccaagcagggacgagctgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccctggc

SEQ ID NO: 14 - DNA, 648 bp, Artificial Sequence
  Light Chain TPP-8236
cagtctgtgctgacacagcctcctagcgcctctggcacacctggccagagagtgaccatcagctgtagcggcagcagctccaacatcggcaacaacgccgtgaactggtatcagcagctgcccggcacagcccccaaactgctgatctacggcaacagcaaccggcccagcggcgtgcccgatagattttccggcagcaagagcggcaccagcgccagcctggctatcagcggactgagaagcgaggacgaggccgactactactgcagcagctacagaggcagcagcaccctggtggtgtttggcggaggcaccaagctgaccgtgctaggccagcctaaagccgcccctagcgtgaccctgttccctccaagcagcgaggaactgcaggccaacaaggccaccctcgtgtgcctgatcagcgacttctatcctggcgccgtgaccgtggcctggaaggccgatagctctcctgtgaaggccggcgtggaaaccaccacccctagcaagcagagcaacaacaaatacgccgccagcagctacctgagcctgacccccgagcagtggaagtcccacagatcctacagctgccaagtgacccacgagggcagcaccgtggaaaagacagtggcccctaccgagtgcagc

SEQ ID NO: 15 - protein, 118 aa, Artificial Sequence
  VH TPP-8237
EVQLLESGGGLVQPGGSLRLSCAASGFTFSSYEMNWVRQAPGKGLEWVSAISSHVGSTYYADSVKGRFTISRDNSKNTLYLQMNSLRAEDTAVYYCVGSTIPTRFDYWGQGTLVTVSS

SEQ ID NO: 16 - protein, 5 aa, Artificial Sequence
  HCDR1 TPP-8237
SYEMN

SEQ ID NO: 17 - protein, 17 aa, Artificial Sequence
  HCDR2 TPP-8237
AISSHVGSTYYADSVKG

SEQ ID NO: 18 - protein, 9 aa, Artificial Sequence
  HCDR3 TPP-8237
STIPTRFDY

SEQ ID NO: 19 - protein, 110 aa, Artificial Sequence
  VL TPP-8237
QSVLTQPPSASGTPGQRVTISCSGSSSNIGRNIVNWYQQLPGTAPKLLIYRNNQRPSGVPDRFSGSKSGTSASLAISGLRSEDEADYYCAAWDDSLNGVVFGGGTKLTVL

SEQ ID NO: 20 - protein, 13 aa, Artificial Sequence
  LCDR1 TPP-8237
SGSSSNIGRNIVN

SEQ ID NO: 21 - protein, 7 aa, Artificial Sequence
  LCDR2 TPP-8237
RNNQRPS

SEQ ID NO: 22 - protein, 11 aa, Artificial Sequence
  LCDR3 TPP-8237
AAWDDSLNGVV

SEQ ID NO: 23 - DNA, 354 bp, Artificial Sequence
  VH TPP-8237
gaagtgcagctgctggaaagcggcggaggcctggtgcagcctggcggatctctgagactgagctgtgccgccagcggcttcaccttcagcagctacgagatgaactgggtgcgccaggcccctggcaagggactggaatgggtgtccgccatcagcagccacgtgggcagcacctactacgccgatagcgtgaagggccggttcaccatcagccgggacaacagcaagaacaccctgtacctgcagatgaacagcctgcgggccgaggacaccgccgtgtactattgtgtgggctccaccatccccaccagattcgactattggggccagggcaccctcgtgaccgtgtcatca

SEQ ID NO: 24 - DNA, 330 bp, Artificial Sequence
  VL TPP-8237
cagtctgtgctgacacagcctcctagcgcctctggcacacctggccagagagtgaccatcagctgtagcggcagcagctccaacatcggccggaacatcgtgaactggtatcagcagctgcccggcaccgcccccaaactgctgatctaccggaacaaccagcggcccagcggcgtgcccgatagattttctggcagcaagagcggcaccagcgccagcctggctatcagcggactgagaagcgaggacgaggccgactactactgcgccgcctgggacgatagcctgaacggcgtggtgtttggcggaggcaccaagctgacagtgcta

SEQ ID NO: 25 - protein, 447 aa, Artificial Sequence
  Heavy Chain TPP-8237
EVQLLESGGGLVQPGGSLRLSCAASGFTFSSYEMNWVRQAPGKGLEWVSAISSHVGSTYYADSVKGRFTISRDNSKNTLYLQMNSLRAEDTAVYYCVGSTIPTRFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 216 aa, Artificial Sequence
  Light Chain TPP-8237
QSVLTQPPSASGTPGQRVTISCSGSSSNIGRNIVNWYQQLPGTAPKLLIYRNNQRPSGVPDRFSGSKSGTSASLAISGLRSEDEADYYCAAWDDSLNGVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 27 - DNA, 1341 bp, Artificial Sequence
  Heavy Chain TPP-8237
gaagtgcagctgctggaaagcggcggaggcctggtgcagcctggcggatctctgagactgagctgtgccgccagcggcttcaccttcagcagctacgagatgaactgggtgcgccaggcccctggcaagggactggaatgggtgtccgccatcagcagccacgtgggcagcacctactacgccgatagcgtgaagggccggttcaccatcagccgggacaacagcaagaacaccctgtacctgcagatgaacagcctgcgggccgaggacaccgccgtgtactattgtgtgggctccaccatccccaccagattcgactattggggccagggcaccctcgtgaccgtgtcatcagccagcaccaagggccccagcgtgttccctctggcccctagcagcaagagcacatctggcggaacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagctctctgggcacccagacctacatctgcaacgtgaaccacaagcccagcaacaccaaggtggacaagaaggtggaacccaagagctgcgacaagacccacacctgtcccccttgtcctgcccccgaactgctgggaggcccttccgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagcctagagaggaacagtacaacagcacctaccgggtggtgtccgtgctgacagtgctgcaccaggactggctgaacggcaaagagtacaagtgcaaggtgtccaacaaggccctgcctgcccccatcgagaaaaccatcagcaaggccaagggccagccccgcgaaccccaggtgtacacactgcccccaagcagggacgagctgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccctggc

SEQ ID NO: 28 - DNA, 648 bp, Artificial Sequence
  Light Chain TPP-8237
cagtctgtgctgacacagcctcctagcgcctctggcacacctggccagagagtgaccatcagctgtagcggcagcagctccaacatcggccggaacatcgtgaactggtatcagcagctgcccggcaccgcccccaaactgctgatctaccggaacaaccagcggcccagcggcgtgcccgatagattttctggcagcaagagcggcaccagcgccagcctggctatcagcggactgagaagcgaggacgaggccgactactactgcgccgcctgggacgatagcctgaacggcgtggtgtttggcggaggcaccaagctgacagtgctaggccagcctaaagccgcccctagcgtgaccctgttccctccaagcagcgaggaactgcaggccaacaaggccaccctcgtgtgcctgatcagcgacttctatcctggcgccgtgaccgtggcctggaaggccgatagctctcctgtgaaggccggcgtggaaaccaccacccctagcaagcagagcaacaacaaatacgccgccagcagctacctgagcctgacccccgagcagtggaagtcccacagatcctacagctgccaagtgacccacgagggcagcaccgtggaaaagacagtggcccctaccgagtgcagc

SEQ ID NO: 29 - protein, 129 aa, Artificial Sequence
  VH TPP-8238
EVQLLESGGGLVQPGGSLRLSCAASGFTFSSYSMNWVRQAPGKGLEWVASISGSGANTYFADTVKGRFTISRDNSKNTLYLQMNSLRAEDTAMYYCARHGQVRLNYGSPTTVYNWFDRWGQGTLVTVSS

SEQ ID NO: 30 - protein, 5 aa, Artificial Sequence
  HCDR1 TPP-8238
SYSMN

SEQ ID NO: 31 - protein, 17 aa, Artificial Sequence
  HCDR2 TPP-8238
SISGSGANTYFADTVKG

SEQ ID NO: 32 - protein, 20 aa, Artificial Sequence
  HCDR3 TPP-8238
HGQVRLNYGSPTTVYNWFDR

SEQ ID NO: 33 - protein, 111 aa, Artificial Sequence
  VL TPP-8238
QSVLTQPPSASGTPGQRVTISCTGSSSNIGAGYDVHWYQQLPGTAPKLLIYGNSNRPSGVPDRFSGSKSGTSASLAISGLRSEDEADYYCAAWHDSLNGWVFGGGTKLTVL

SEQ ID NO: 34 - protein, 14 aa, Artificial Sequence
  LCDR1 TPP-8238
TGSSSNIGAGYDVH

SEQ ID NO: 35 - protein, 7 aa, Artificial Sequence
  LCDR2 TPP-8238
GNSNRPS

SEQ ID NO: 36 - protein, 11 aa, Artificial Sequence
  LCDR3 TPP-8238
AAWHDSLNGWV

SEQ ID NO: 37 - DNA, 387 bp, Artificial Sequence
  VH TPP-8238
gaagtgcagctgctggaaagcggcggaggcctggtgcagcctggcggatctctgagactgagctgtgccgccagcggcttcaccttcagcagctacagcatgaactgggtgcgccaggcccctggcaagggactggaatgggtggccagcatctctggcagcggcgccaatacctacttcgccgacaccgtgaagggccggttcaccatctcccgggacaacagcaagaacaccctgtacctgcagatgaacagcctgcgggccgaggacaccgccatgtactactgtgccagacacggccaagtgcggctgaactacggcagccctaccaccgtgtacaattggttcgacagatggggccagggcaccctcgtgacagtgtcatca

SEQ ID NO: 38 - DNA, 333 bp, Artificial Sequence
  VL TPP-8238
cagtctgtgctgacacagcctcctagcgcctctggcacacctggccagagagtgaccatcagctgtaccggcagcagctccaacatcggagccggctatgacgtgcactggtatcagcagctgcctggcaccgcccccaaactgctgatctacggcaacagcaaccggcccagcggcgtgcccgatagattttccggcagcaagagcggcaccagcgccagcctggctatcagcggactgagaagcgaggacgaggccgactactactgtgccgcctggcacgacagcctgaacggctgggtgttcggcggaggcaccaagctgacagtgcta

SEQ ID NO: 39 - protein, 458 aa, Artificial Sequence
  Heavy Chain TPP-8238
EVQLLESGGGLVQPGGSLRLSCAASGFTFSSYSMNWVRQAPGKGLEWVASISGSGANTYFADTVKGRFTISRDNSKNTLYLQMNSLRAEDTAMYYCARHGQVRLNYGSPTTVYNWFDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - protein, 217 aa, Artificial Sequence
  Light Chain TPP-8238
QSVLTQPPSASGTPGQRVTISCTGSSSNIGAGYDVHWYQQLPGTAPKLLIYGNSNRPSGVPDRFSGSKSGTSASLAISGLRSEDEADYYCAAWHDSLNGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 41 - DNA, 1374 bp, Artificial Sequence
  Heavy Chain TPP-8238
gaagtgcagctgctggaaagcggcggaggcctggtgcagcctggcggatctctgagactgagctgtgccgccagcggcttcaccttcagcagctacagcatgaactgggtgcgccaggcccctggcaagggactggaatgggtggccagcatctctggcagcggcgccaatacctacttcgccgacaccgtgaagggccggttcaccatctcccgggacaacagcaagaacaccctgtacctgcagatgaacagcctgcgggccgaggacaccgccatgtactactgtgccagacacggccaagtgcggctgaactacggcagccctaccaccgtgtacaattggttcgacagatggggccagggcaccctcgtgacagtgtcatcagccagcaccaagggccccagcgtgttccctctggcccctagcagcaagagcacatctggcggaacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagctctctgggcacccagacctacatctgcaacgtgaaccacaagcccagcaacaccaaggtggacaagaaggtggaacccaagagctgcgacaagacccacacctgtcccccttgtcctgcccccgaactgctgggaggcccttccgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagcctagagaggaacagtacaacagcacctaccgggtggtgtccgtgctgacagtgctgcaccaggactggctgaacggcaaagagtacaagtgcaaggtgtccaacaaggccctgcctgcccccatcgagaaaaccatcagcaaggccaagggccagccccgcgaaccccaggtgtacacactgcccccaagcagggacgagctgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccctggc

SEQ ID NO: 42 - DNA, 651 bp, Artificial Sequence
  Light Chain TPP-8238
cagtctgtgctgacacagcctcctagcgcctctggcacacctggccagagagtgaccatcagctgtaccggcagcagctccaacatcggagccggctatgacgtgcactggtatcagcagctgcctggcaccgcccccaaactgctgatctacggcaacagcaaccggcccagcggcgtgcccgatagattttccggcagcaagagcggcaccagcgccagcctggctatcagcggactgagaagcgaggacgaggccgactactactgtgccgcctggcacgacagcctgaacggctgggtgttcggcggaggcaccaagctgacagtgctaggccagcctaaagccgcccctagcgtgaccctgttccctccaagcagcgaggaactgcaggccaacaaggccaccctcgtgtgcctgatcagcgacttctatcctggcgccgtgaccgtggcctggaaggccgatagctctcctgtgaaggccggcgtggaaaccaccacccctagcaagcagagcaacaacaaatacgccgccagcagctacctgagcctgacccccgagcagtggaagtcccacagatcctacagctgccaagtgacccacgagggcagcaccgtggaaaagacagtggcccctaccgagtgcagc

SEQ ID NO: 43 - protein, 121 aa, Artificial Sequence
  VH TPP-8239
EVQLLESGGGLVQPGGSLRLSCAASGFTFSSYAMSWVRQAPGKGLEWVSIIYGGGPTYYADSVKGRFTISRDNSKNTLYLQMNSLRAEDTAVYYCAKGGPQATGIRRMDVWGQGTLVTVSS

SEQ ID NO: 44 - protein, 5 aa, Artificial Sequence
  HCDR1 TPP-8239
SYAMS

SEQ ID NO: 45 - protein, 16 aa, Artificial Sequence
  HCDR2 TPP-8239
IIYGGGPTYYADSVKG

SEQ ID NO: 46 - protein, 13 aa, Artificial Sequence
  HCDR3 TPP-8239
GGPQATGIRRMDV

SEQ ID NO: 47 - protein, 110 aa, Artificial Sequence
  VL TPP-8239
QSVLTQPPSASGTPGQRVTISCSGSSSNIGSNYVYWYQQLPGTAPKLLIYDNSNRPSGVPDRFSGSKSGTSASLAISGLRSEDEADYYCAAWDDSLSGRVFGGGTKLTVL

SEQ ID NO: 48 - protein, 13 aa, Artificial Sequence
  LCDR1 TPP-8239
SGSSSNIGSNYVY

SEQ ID NO: 49 - protein, 7 aa, Artificial Sequence
  LCDR2 TPP-8239
DNSNRPS

SEQ ID NO: 50 - protein, 11 aa, Artificial Sequence
  LCDR3 TPP-8239
AAWDDSLSGRV

SEQ ID NO: 51 - DNA, 363 bp, Artificial Sequence
  VH TPP-8239
gaagtgcagctgctggaaagcggcggaggcctggtgcagcctggcggatctctgagactgagctgtgccgccagcggcttcaccttcagcagctacgccatgagctgggtgcgccaggcccctggaaaaggcctggaatgggtgtccatcatctatggcggcggacccacctactacgccgacagcgtgaagggccggttcaccatctcccgggacaacagcaagaacaccctgtacctgcagatgaacagcctgcgggccgaggacaccgccgtgtactattgtgccaagggcggacctcaggccaccggcatcagacggatggatgtgtggggccagggcaccctcgtgacagtgtcatca

SEQ ID NO: 52 - DNA, 330 bp, Artificial Sequence
  VL TPP-8239
cagtctgtgctgacacagcctcctagcgcctctggcacacctggccagagagtgaccatcagctgtagcggcagcagctccaacatcggcagcaactacgtgtactggtatcagcagctgcccggcaccgcccccaaactgctgatctacgacaacagcaaccggcccagcggcgtgcccgatagattttccggcagcaagagcggcaccagcgccagcctggctatcagcggactgagaagcgaggacgaggccgactactactgcgccgcctgggacgatagcctgagcggcagagtgtttggcggaggcaccaagctgaccgtgcta

SEQ ID NO: 53 - protein, 450 aa, Artificial Sequence
  Heavy Chain TPP-8239
EVQLLESGGGLVQPGGSLRLSCAASGFTFSSYAMSWVRQAPGKGLEWVSIIYGGGPTYYADSVKGRFTISRDNSKNTLYLQMNSLRAEDTAVYYCAKGGPQATGIRRMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 54 - protein, 216 aa, Artificial Sequence
  Light Chain TPP-8239
QSVLTQPPSASGTPGQRVTISCSGSSSNIGSNYVYWYQQLPGTAPKLLIYDNSNRPSGVPDRFSGSKSGTSASLAISGLRSEDEADYYCAAWDDSLSGRVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 55 - DNA, 1350 bp, Artificial Sequence
  Heavy Chain TPP-8239
gaagtgcagctgctggaaagcggcggaggcctggtgcagcctggcggatctctgagactgagctgtgccgccagcggcttcaccttcagcagctacgccatgagctgggtgcgccaggcccctggaaaaggcctggaatgggtgtccatcatctatggcggcggacccacctactacgccgacagcgtgaagggccggttcaccatctcccgggacaacagcaagaacaccctgtacctgcagatgaacagcctgcgggccgaggacaccgccgtgtactattgtgccaagggcggacctcaggccaccggcatcagacggatggatgtgtggggccagggcaccctcgtgacagtgtcatcagccagcaccaagggccccagcgtgttccctctggcccctagcagcaagagcacatctggcggaacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagctctctgggcacccagacctacatctgcaacgtgaaccacaagcccagcaacaccaaggtggacaagaaggtggaacccaagagctgcgacaagacccacacctgtcccccttgtcctgcccccgaactgctgggaggcccttccgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagcctagagaggaacagtacaacagcacctaccgggtggtgtccgtgctgacagtgctgcaccaggactggctgaacggcaaagagtacaagtgcaaggtgtccaacaaggccctgcctgcccccatcgagaaaaccatcagcaaggccaagggccagccccgcgaaccccaggtgtacacactgcccccaagcagggacgagctgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccctggc

SEQ ID NO: 56 - DNA, 648 bp, Artificial Sequence
  Light Chain TPP-8239
cagtctgtgctgacacagcctcctagcgcctctggcacacctggccagagagtgaccatcagctgtagcggcagcagctccaacatcggcagcaactacgtgtactggtatcagcagctgcccggcaccgcccccaaactgctgatctacgacaacagcaaccggcccagcggcgtgcccgatagattttccggcagcaagagcggcaccagcgccagcctggctatcagcggactgagaagcgaggacgaggccgactactactgcgccgcctgggacgatagcctgagcggcagagtgtttggcggaggcaccaagctgaccgtgctaggccagcctaaagccgcccctagcgtgaccctgttccctccaagcagcgaggaactgcaggccaacaaggccaccctcgtgtgcctgatcagcgacttctatcctggcgccgtgaccgtggcctggaaggccgatagctctcctgtgaaggccggcgtggaaaccaccacccctagcaagcagagcaacaacaaatacgccgccagcagctacctgagcctgacccccgagcagtggaagtcccacagatcctacagctgccaagtgacccacgagggcagcaccgtggaaaagacagtggcccctaccgagtgcagc

SEQ ID NO: 57 - protein, 120 aa, Artificial Sequence
  VH TPP-8240
EVQLLESGGGLVQPGGSLRLSCAASGFTFSSYAMSWVRQAPGKGLEWVAVISYDGSNKHYADSVKGRFTISRDNSKNTLYLQMNSLRAEDTAVYYCARDSMVRGKIFDYWGQGTLVTVSS

SEQ ID NO: 58 - protein, 5 aa, Artificial Sequence
  HCDR1 TPP-8240
SYAMS

SEQ ID NO: 59 - protein, 17 aa, Artificial Sequence
  HCDR2 TPP-8240
VISYDGSNKHYADSVKG

SEQ ID NO: 60 - protein, 11 aa, Artificial Sequence
  HCDR3 TPP-8240
DSMVRGKIFDY

SEQ ID NO: 61 - protein, 108 aa, Artificial Sequence
  VL TPP-8240
QSVLTQPPSASGTPGQRVTISCSGSSSNIGNNAVNWYQQLPGTAPKLLIYEDNKRPSGVPDRFSGSKSGTSASLAISGLRSEDEADYYCMIWHSSAWVFGGGTKLTVL

SEQ ID NO: 62 - protein, 13 aa, Artificial Sequence
  LCDR1 TPP-8240
SGSSSNIGNNAVN

SEQ ID NO: 63 - protein, 7 aa, Artificial Sequence
  LCDR2 TPP-8240
EDNKRPS

SEQ ID NO: 64 - protein, 9 aa, Artificial Sequence
  LCDR2 TPP-8240
MIWHSSAWV

SEQ ID NO: 65 - DNA, 360 bp, Artificial Sequence
  VH TPP-8240
gaagtgcagctgctggaaagcggcggaggcctggtgcagcctggcggatctctgagactgagctgtgccgccagcggcttcaccttcagcagctacgccatgagctgggtgcgccaggcccctggaaaaggcctggaatgggtggccgtgatcagctacgacggcagcaacaagcactacgccgacagcgtgaagggccggttcaccatctcccgggacaacagcaagaacaccctgtacctgcagatgaacagcctgcgggccgaggacaccgccgtgtactactgtgccagagacagcatggtgcgcggcaagatcttcgactactggggccagggcaccctcgtgacagtgtcatca

SEQ ID NO: 66 - DNA, 324 bp, Artificial Sequence
  VL TPP-8240
cagtctgtgctgacacagcctcctagcgcctctggcacacctggccagagagtgaccatcagctgtagcggcagcagctccaacatcggcaacaacgccgtgaactggtatcagcagctgcccggcacagcccccaaactgctgatctacgaggacaacaagcggcccagcggcgtgcccgatagattcagcggctctaagagcggcaccagcgccagcctggccatctctggactgagaagcgaggacgaggccgactactactgcatgatctggcacagcagcgcctgggtgttcggcggaggcacaaagctgacagtgcta

SEQ ID NO: 67 - protein, 449 aa, Artificial Sequence
  Heavy Chain TPP-8240
EVQLLESGGGLVQPGGSLRLSCAASGFTFSSYAMSWVRQAPGKGLEWVAVISYDGSNKHYADSVKGRFTISRDNSKNTLYLQMNSLRAEDTAVYYCARDSMVRGKIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 68 - protein, 214 aa, Artificial Sequence
  Light Chain TPP-8240
QSVLTQPPSASGTPGQRVTISCSGSSSNIGNNAVNWYQQLPGTAPKLLIYEDNKRPSGVPDRFSGSKSGTSASLAISGLRSEDEADYYCMIWHSSAWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 69 - DNA, 1347 bp, Artificial Sequence
  Heavy Chain TPP-8240
gaagtgcagctgctggaaagcggcggaggcctggtgcagcctggcggatctctgagactgagctgtgccgccagcggcttcaccttcagcagctacgccatgagctgggtgcgccaggcccctggaaaaggcctggaatgggtggccgtgatcagctacgacggcagcaacaagcactacgccgacagcgtgaagggccggttcaccatctcccgggacaacagcaagaacaccctgtacctgcagatgaacagcctgcgggccgaggacaccgccgtgtactactgtgccagagacagcatggtgcgcggcaagatcttcgactactggggccagggcaccctcgtgacagtgtcatcagccagcaccaagggccccagcgtgttccctctggcccctagcagcaagagcacatctggcggaacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagctctctgggcacccagacctacatctgcaacgtgaaccacaagcccagcaacaccaaggtggacaagaaggtggaacccaagagctgcgacaagacccacacctgtcccccttgtcctgcccccgaactgctgggaggcccttccgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagcctagagaggaacagtacaacagcacctaccgggtggtgtccgtgctgacagtgctgcaccaggactggctgaacggcaaagagtacaagtgcaaggtgtccaacaaggccctgcctgcccccatcgagaaaaccatcagcaaggccaagggccagccccgcgaaccccaggtgtacacactgcccccaagcagggacgagctgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccctggc

SEQ ID NO: 70 - DNA, 642 bp, Artificial Sequence
  Light Chain TPP-8240
cagtctgtgctgacacagcctcctagcgcctctggcacacctggccagagagtgaccatcagctgtagcggcagcagctccaacatcggcaacaacgccgtgaactggtatcagcagctgcccggcacagcccccaaactgctgatctacgaggacaacaagcggcccagcggcgtgcccgatagattcagcggctctaagagcggcaccagcgccagcctggccatctctggactgagaagcgaggacgaggccgactactactgcatgatctggcacagcagcgcctgggtgttcggcggaggcacaaagctgacagtgctaggccagcctaaagccgcccctagcgtgaccctgttccctccaagcagcgaggaactgcaggccaacaaggccaccctcgtgtgcctgatcagcgacttctatcctggcgccgtgaccgtggcctggaaggccgatagctctcctgtgaaggccggcgtggaaaccaccacccctagcaagcagagcaacaacaaatacgccgccagcagctacctgagcctgacccccgagcagtggaagtcccacagatcctacagctgccaagtgacccacgagggcagcaccgtggaaaagacagtggcccctaccgagtgcagc

SEQ ID NO: 71 - protein, 127 aa, Artificial Sequence
  VH TPP-8241
EVQLLESGGGLVQPGGSLRLSCAASGFTFNKFAMHWVRQAPGKGLEWVASIGSSRRYLYYSDSVKGRFTISRDNSKNTLYLQMNSLRAEDTAVYYCARDRGYYDILTGYVWDAFDIRGQGTLVTVSS

SEQ ID NO: 72 - protein, 5 aa, Artificial Sequence
  HCDR1 TPP-8241
KFAMH

SEQ ID NO: 73 - protein, 17 aa, Artificial Sequence
  HCDR2 TPP-8241
SIGSSRRYLYYSDSVKG

SEQ ID NO: 74 - protein, 18 aa, Artificial Sequence
  HCDR3 TPP-8241
DRGYYDILTGYVWDAFDI

SEQ ID NO: 75 - protein, 110 aa, Artificial Sequence
  VL TPP-8241
QSVLTQPPSASGTPGQRVTISCSGSSSNIGNNYVSWYQQLPGTAPKLLIYGNSNRPSGVPDRFSGSKSGTSASLAISGLRSEDEADYYCAAWDDSLNGWVFGGGTKLTVL

SEQ ID NO: 76 - protein, 13 aa, Artificial Sequence
  LCDR1 TPP-8241
SGSSSNIGNNYVS

SEQ ID NO: 77 - protein, 7 aa, Artificial Sequence
  LCDR2 TPP-8241
GNSNRPS

SEQ ID NO: 78 - protein, 11 aa, Artificial Sequence
  LCDR3 TPP-8241
AAWDDSLNGWV

SEQ ID NO: 79 - DNA, 381 bp, Artificial Sequence
  VH TPP-8241
gaagtgcagctgctggaaagcggcggaggcctggtgcagcctggcggatctctgagactgagctgtgccgccagcggcttcaccttcaacaagttcgccatgcactgggtgcgccaggcccctggaaaaggcctggaatgggtggccagcatcggcagctccagaagatacctgtactacagcgacagcgtgaagggccggttcaccatcagccgggacaacagcaagaacaccctgtacctgcagatgaacagcctgcgggccgaggacaccgccgtgtactactgcgctagagacagaggctactacgacatcctgaccggctacgtgtgggacgccttcgacatcagaggccagggcacactcgtgaccgtgtcctca

SEQ ID NO: 80 - DNA, 330 bp, Artificial Sequence
  VL TPP-8241
cagtctgtgctgacacagcctcctagcgcctctggcacacctggccagagagtgaccatcagctgtagcggcagcagctccaacatcggcaacaactacgtgtcctggtatcagcagctgcccggcaccgcccccaaactgctgatctacggcaacagcaaccggcccagcggcgtgcccgatagattttccggcagcaagagcggcaccagcgccagcctggctatcagcggactgagaagcgaggacgaggccgactactactgcgccgcctgggacgatagcctgaacggctgggtgttcggcggaggcaccaagctgacagtgcta

SEQ ID NO: 81 - protein, 456 aa, Artificial Sequence
  Heavy Chain TPP-8241
EVQLLESGGGLVQPGGSLRLSCAASGFTFNKFAMHWVRQAPGKGLEWVASIGSSRRYLYYSDSVKGRFTISRDNSKNTLYLQMNSLRAEDTAVYYCARDRGYYDILTGYVWDAFDIRGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 82 - protein, 216 aa, Artificial Sequence
  Light Chain TPP-8241
QSVLTQPPSASGTPGQRVTISCSGSSSNIGNNYVSWYQQLPGTAPKLLIYGNSNRPSGVPDRFSGSKSGTSASLAISGLRSEDEADYYCAAWDDSLNGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 83 - DNA, 1368 bp, Artificial Sequence
  Heavy Chain TPP-8241
gaagtgcagctgctggaaagcggcggaggcctggtgcagcctggcggatctctgagactgagctgtgccgccagcggcttcaccttcaacaagttcgccatgcactgggtgcgccaggcccctggaaaaggcctggaatgggtggccagcatcggcagctccagaagatacctgtactacagcgacagcgtgaagggccggttcaccatcagccgggacaacagcaagaacaccctgtacctgcagatgaacagcctgcgggccgaggacaccgccgtgtactactgcgctagagacagaggctactacgacatcctgaccggctacgtgtgggacgccttcgacatcagaggccagggcacactcgtgaccgtgtcctcagccagcaccaagggccccagcgtgttccctctggcccctagcagcaagagcacatctggcggaacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagctctctgggcacccagacctacatctgcaacgtgaaccacaagcccagcaacaccaaggtggacaagaaggtggaacccaagagctgcgacaagacccacacctgtcccccttgtcctgcccccgaactgctgggaggcccttccgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagcctagagaggaacagtacaacagcacctaccgggtggtgtccgtgctgacagtgctgcaccaggactggctgaacggcaaagagtacaagtgcaaggtgtccaacaaggccctgcctgcccccatcgagaaaaccatcagcaaggccaagggccagccccgcgaaccccaggtgtacacactgcccccaagcagggacgagctgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccctggc

SEQ ID NO: 84 - DNA, 648 bp, Artificial Sequence
  Light Chain TPP-8241
cagtctgtgctgacacagcctcctagcgcctctggcacacctggccagagagtgaccatcagctgtagcggcagcagctccaacatcggcaacaactacgtgtcctggtatcagcagctgcccggcaccgcccccaaactgctgatctacggcaacagcaaccggcccagcggcgtgcccgatagattttccggcagcaagagcggcaccagcgccagcctggctatcagcggactgagaagcgaggacgaggccgactactactgcgccgcctgggacgatagcctgaacggctgggtgttcggcggaggcaccaagctgacagtgctaggccagcctaaagccgcccctagcgtgaccctgttccctccaagcagcgaggaactgcaggccaacaaggccaccctcgtgtgcctgatcagcgacttctatcctggcgccgtgaccgtggcctggaaggccgatagctctcctgtgaaggccggcgtggaaaccaccacccctagcaagcagagcaacaacaaatacgccgccagcagctacctgagcctgacccccgagcagtggaagtcccacagatcctacagctgccaagtgacccacgagggcagcaccgtggaaaagacagtggcccctaccgagtgcagc

SEQ ID NO: 85 - protein, 122 aa, Artificial Sequence
  VH TPP-8243
EVQLLESGGGLVQPGGSLRLSCAASGFTFSNYGIHWVRQAPGKGLEWVSAIGTGGDTYYADSVMGRFTISRDNSKNTLYLQMNSLRAEDTAVYYCARDRGYYDILTGLYDYWGQGTLVTVSS

SEQ ID NO: 86 - protein, 5 aa, Artificial Sequence
  HCDR1 TPP-8243
NYGIH

SEQ ID NO: 87 - protein, 16 aa, Artificial Sequence
  HCDR2 TPP-8243
AIGTGGDTYYADSVMG

SEQ ID NO: 88 - protein, 14 aa, Artificial Sequence
  HCDR3 TPP-8243
DRGYYDILTGLYDY

SEQ ID NO: 89 - protein, 109 aa, Artificial Sequence
  VL TPP-8243
QSVLTQPPSASGTPGQRVTISCSGSSSNIGNNAVNWYQQLPGTAPKLLIYGNYHRPSGVPDRFSGSKSGTSASLAISGLRSEDEADYYCSSYAGSNNWVFGGGTKLTVL

SEQ ID NO: 90 - protein, 13 aa, Artificial Sequence
  LCDR1 TPP-8243
SGSSSNIGNNAVN

SEQ ID NO: 91 - protein, 7 aa, Artificial Sequence
  LCDR2 TPP-8243
GNYHRPS

SEQ ID NO: 92 - protein, 10 aa, Artificial Sequence
  LCDR3 TPP-8243
SSYAGSNNWV

SEQ ID NO: 93 - DNA, 366 bp, Artificial Sequence
  VH TPP-8243
gaagtgcagctgctggaaagcggcggaggcctggtgcagcctggcggatctctgagactgagctgtgccgccagcggcttcaccttcagcaactacggcatccactgggtgcgccaggcccctggaaaaggcctggaatgggtgtccgccatcggcacaggcggcgatacctactacgccgacagcgtgatgggccggttcaccatcagccgggacaacagcaagaacaccctgtacctgcagatgaacagcctgcgggccgaggacaccgccgtgtactactgcgctagagacagaggctactacgacatcctgaccggcctgtacgactactggggccagggaacactcgtgaccgtgtcctca

SEQ ID NO: 94 - DNA, 327 bp, Artificial Sequence
  VL TPP-8243
cagtctgtgctgacacagcctcctagcgcctctggcacacctggccagagagtgaccatcagctgtagcggcagcagctccaacatcggcaacaacgccgtgaactggtatcagcagctgcccggcacagcccccaaactgctgatctacggcaactaccaccggcccagcggcgtgcccgatagattttctggcagcaagagcggcaccagcgccagcctggctatcagcggactgagaagcgaggacgaggccgactactactgcagcagctacgccggcagcaacaactgggtgttcggcggaggcaccaagctgaccgtgcta

SEQ ID NO: 95 - protein, 451 aa, Artificial Sequence
  Heavy Chain TPP-8243
EVQLLESGGGLVQPGGSLRLSCAASGFTFSNYGIHWVRQAPGKGLEWVSAIGTGGDTYYADSVMGRFTISRDNSKNTLYLQMNSLRAEDTAVYYCARDRGYYDILTGLYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 96 - protein, 215 aa, Artificial Sequence
  Light Chain TPP-8243
QSVLTQPPSASGTPGQRVTISCSGSSSNIGNNAVNWYQQLPGTAPKLLIYGNYHRPSGVPDRFSGSKSGTSASLAISGLRSEDEADYYCSSYAGSNNWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 97 - DNA, 1353 bp, Artificial Sequence
  Heavy Chain TPP-8243
gaagtgcagctgctggaaagcggcggaggcctggtgcagcctggcggatctctgagactgagctgtgccgccagcggcttcaccttcagcaactacggcatccactgggtgcgccaggcccctggaaaaggcctggaatgggtgtccgccatcggcacaggcggcgatacctactacgccgacagcgtgatgggccggttcaccatcagccgggacaacagcaagaacaccctgtacctgcagatgaacagcctgcgggccgaggacaccgccgtgtactactgcgctagagacagaggctactacgacatcctgaccggcctgtacgactactggggccagggaacactcgtgaccgtgtcctcagccagcaccaagggccccagcgtgttccctctggcccctagcagcaagagcacatctggcggaacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagctctctgggcacccagacctacatctgcaacgtgaaccacaagcccagcaacaccaaggtggacaagaaggtggaacccaagagctgcgacaagacccacacctgtcccccttgtcctgcccccgaactgctgggaggcccttccgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagcctagagaggaacagtacaacagcacctaccgggtggtgtccgtgctgacagtgctgcaccaggactggctgaacggcaaagagtacaagtgcaaggtgtccaacaaggccctgcctgcccccatcgagaaaaccatcagcaaggccaagggccagccccgcgaaccccaggtgtacacactgcccccaagcagggacgagctgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccctggc

SEQ ID NO: 98 - DNA, 645 bp, Artificial Sequence
  Light Chain TPP-8243
cagtctgtgctgacacagcctcctagcgcctctggcacacctggccagagagtgaccatcagctgtagcggcagcagctccaacatcggcaacaacgccgtgaactggtatcagcagctgcccggcacagcccccaaactgctgatctacggcaactaccaccggcccagcggcgtgcccgatagattttctggcagcaagagcggcaccagcgccagcctggctatcagcggactgagaagcgaggacgaggccgactactactgcagcagctacgccggcagcaacaactgggtgttcggcggaggcaccaagctgaccgtgctaggccagcctaaagccgcccctagcgtgaccctgttccctccaagcagcgaggaactgcaggccaacaaggccaccctcgtgtgcctgatcagcgacttctatcctggcgccgtgaccgtggcctggaaggccgatagctctcctgtgaaggccggcgtggaaaccaccacccctagcaagcagagcaacaacaaatacgccgccagcagctacctgagcctgacccccgagcagtggaagtcccacagatcctacagctgccaagtgacccacgagggcagcaccgtggaaaagacagtggcccctaccgagtgcagc

SEQ ID NO: 99 - protein, 123 aa, Artificial Sequence
  VH TPP-8246
EVQLLESGGGLVQPGGSLRLSCAASGFTFSSYSMNWVRQAPGKGLEWVSTISGSGGITHYADSVKGRFTISRDNSKNTLYLQMNSLRAEDTAVYYCARDRGNRVAARPAFDYWGQGTLVTVSS

SEQ ID NO: 100 - protein, 5 aa, Artificial Sequence
  HCDR1 TPP-8246
SYSMN

SEQ ID NO: 101 - protein, 17 aa, Artificial Sequence
  HCDR2 TPP-8246
TISGSGGITHYADSVKG

SEQ ID NO: 102 - protein, 14 aa, Artificial Sequence
  HCDR3 TPP-8246
DRGNRVAARPAFDY

SEQ ID NO: 103 - protein, 111 aa, Artificial Sequence
  VL TPP-8246
QSVLTQPPSASGTPGQRVTISCTESSSNTGAGYDVHWYQQLPGTAPKLLIYGNTNRPSGVPDRFSGSKSGTSASLAISGLRSEDEADYYCASWDDNLGGWVFGGGTKLTVL

SEQ ID NO: 104 - protein, 14 aa, Artificial Sequence
  LCDR1 TPP-8246
TESSSNTGAGYDVH

SEQ ID NO: 105 - protein, 7 aa, Artificial Sequence
  LCDR2 TPP-8246
GNTNRPS

SEQ ID NO: 106 - protein, 11 aa, Artificial Sequence
  LCDR3 TPP-8246
ASWDDNLGGWV

SEQ ID NO: 107 - DNA, 369 bp, Artificial Sequence
  VH TPP-8246
gaagtgcagctgctggaaagcggcggaggcctggtgcagcctggcggatctctgagactgagctgtgccgccagcggcttcaccttcagcagctacagcatgaactgggtgcgccaggcccctggcaagggactggaatgggtgtccacaatcagcggcagcggcggcatcacccactacgccgattctgtgaagggccggttcaccatcagccgggacaacagcaagaacaccctgtacctgcagatgaacagcctgcgggccgaggacaccgccgtgtactactgcgctagagacagaggcaaccgggtggccgccagacccgcctttgattattggggccagggcaccctcgtgaccgtgtcatca

SEQ ID NO: 108 - DNA, 333 bp, Artificial Sequence
  VL TPP-8246
cagtctgtgctgacacagcctcctagcgcctctggcacacctggccagagagtgaccatcagctgcaccgagagcagctccaacaccggcgctggctacgacgtgcactggtatcagcagctgcctggcaccgcccccaaactgctgatctacggcaacaccaaccggcccagcggcgtgcccgatagattttccggcagcaagagcggcaccagcgccagcctggctatcagcggactgagaagcgaggacgaggccgactactactgcgccagctgggacgataatctgggcggctgggtgttcggcggaggcaccaaactgacagtgcta

SEQ ID NO: 109 - protein, 452 aa, Artificial Sequence
  Heavy Chain TPP-8246
EVQLLESGGGLVQPGGSLRLSCAASGFTFSSYSMNWVRQAPGKGLEWVSTISGSGGITHYADSVKGRFTISRDNSKNTLYLQMNSLRAEDTAVYYCARDRGNRVAARPAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 110 - protein, 217 aa, Artificial Sequence
  Light Chain TPP-8246
QSVLTQPPSASGTPGQRVTISCTESSSNTGAGYDVHWYQQLPGTAPKLLIYGNTNRPSGVPDRFSGSKSGTSASLAISGLRSEDEADYYCASWDDNLGGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 111 - DNA, 1356 bp, Artificial Sequence
  Heavy Chain TPP-8246
gaagtgcagctgctggaaagcggcggaggcctggtgcagcctggcggatctctgagactgagctgtgccgccagcggcttcaccttcagcagctacagcatgaactgggtgcgccaggcccctggcaagggactggaatgggtgtccacaatcagcggcagcggcggcatcacccactacgccgattctgtgaagggccggttcaccatcagccgggacaacagcaagaacaccctgtacctgcagatgaacagcctgcgggccgaggacaccgccgtgtactactgcgctagagacagaggcaaccgggtggccgccagacccgcctttgattattggggccagggcaccctcgtgaccgtgtcatcagccagcaccaagggccccagcgtgttccctctggcccctagcagcaagagcacatctggcggaacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagctctctgggcacccagacctacatctgcaacgtgaaccacaagcccagcaacaccaaggtggacaagaaggtggaacccaagagctgcgacaagacccacacctgtcccccttgtcctgcccccgaactgctgggaggcccttccgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagcctagagaggaacagtacaacagcacctaccgggtggtgtccgtgctgacagtgctgcaccaggactggctgaacggcaaagagtacaagtgcaaggtgtccaacaaggccctgcctgcccccatcgagaaaaccatcagcaaggccaagggccagccccgcgaaccccaggtgtacacactgcccccaagcagggacgagctgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccctggc

SEQ ID NO: 112 - DNA, 651 bp, Artificial Sequence
  Light Chain TPP-8246
cagtctgtgctgacacagcctcctagcgcctctggcacacctggccagagagtgaccatcagctgcaccgagagcagctccaacaccggcgctggctacgacgtgcactggtatcagcagctgcctggcaccgcccccaaactgctgatctacggcaacaccaaccggcccagcggcgtgcccgatagattttccggcagcaagagcggcaccagcgccagcctggctatcagcggactgagaagcgaggacgaggccgactactactgcgccagctgggacgataatctgggcggctgggtgttcggcggaggcaccaaactgacagtgctaggccagcctaaagccgcccctagcgtgaccctgttccctccaagcagcgaggaactgcaggccaacaaggccaccctcgtgtgcctgatcagcgacttctatcctggcgccgtgaccgtggcctggaaggccgatagctctcctgtgaaggccggcgtggaaaccaccacccctagcaagcagagcaacaacaaatacgccgccagcagctacctgagcctgacccccgagcagtggaagtcccacagatcctacagctgccaagtgacccacgagggcagcaccgtggaaaagacagtggcccctaccgagtgcagc

SEQ ID NO: 113 - protein, 122 aa, Artificial Sequence; identical to SEQ ID NO: 85
  VH TPP-9238

SEQ ID NO: 114 - protein, 5 aa, Artificial Sequence
  HCDR1 TPP-9238
NYGIH

SEQ ID NO: 115 - protein, 16 aa, Artificial Sequence
  HCDR2 TPP-9238
AIGTGGDTYYADSVMG

SEQ ID NO: 116 - protein, 14 aa, Artificial Sequence
  HCDR3 TPP-9238
DRGYYDILTGLYDY

SEQ ID NO: 117 - protein, 109 aa, Artificial Sequence
  VL TPP-9238
QSVLTQPPSASGTPGQRVTISCSGSSSNIGNNYVNWYQQLPGTAPKLLIYGNYHRPSGVPDRFSGSKSGTSASLAITGLQAEDEADYYCSSYAGSNNWVFGGGTKLTVL

SEQ ID NO: 118 - protein, 13 aa, Artificial Sequence
  LCDR1 TPP-9238
SGSSSNIGNNYVN

SEQ ID NO: 119 - protein, 7 aa, Artificial Sequence
  LCDR2 TPP-9238
GNYHRPS

SEQ ID NO: 120 - protein, 10 aa, Artificial Sequence
  LCDR3 TPP-9238
SSYAGSNNWV

SEQ ID NO: 121 - DNA, 366 bp, Artificial Sequence; identical to SEQ ID NO: 93
  VH TPP-9238

SEQ ID NO: 122 - DNA, 327 bp, Artificial Sequence
  VL TPP-9238
cagtctgtgctgacacagcctcctagcgcctctggcacacctggccagagagtgaccatcagctgtagcggcagcagctccaacatcggcaacaactacgtgaactggtatcagcagctgcccggcaccgcccccaaactgctgatctacggcaactaccaccggcccagcggcgtgcccgatagattttctggcagcaagagcggcaccagcgccagcctggctattactggactgcaggccgaggacgaggccgactactactgtagcagctacgccggcagcaacaactgggtgttcggcggaggcaccaagctgaccgtgcta

SEQ ID NO: 123 - protein, 451 aa, Artificial Sequence; identical to SEQ ID NO: 95
  Heavy Chain TPP-9238

SEQ ID NO: 124 - protein, 215 aa, Artificial Sequence
  Light Chain TPP-9238
QSVLTQPPSASGTPGQRVTISCSGSSSNIGNNYVNWYQQLPGTAPKLLIYGNYHRPSGVPDRFSGSKSGTSASLAITGLQAEDEADYYCSSYAGSNNWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 125 - DNA, 1353 bp, Artificial Sequence; identical to SEQ ID NO: 97
  Heavy Chain TPP-9238

SEQ ID NO: 126 - DNA, 645 bp, Artificial Sequence
  Light Chain TPP-9238
cagtctgtgctgacacagcctcctagcgcctctggcacacctggccagagagtgaccatcagctgtagcggcagcagctccaacatcggcaacaactacgtgaactggtatcagcagctgcccggcaccgcccccaaactgctgatctacggcaactaccaccggcccagcggcgtgcccgatagattttctggcagcaagagcggcaccagcgccagcctggctattactggactgcaggccgaggacgaggccgactactactgtagcagctacgccggcagcaacaactgggtgttcggcggaggcaccaagctgaccgtgctaggccagcctaaagccgcccctagcgtgaccctgttccctccaagcagcgaggaactgcaggccaacaaggccaccctcgtgtgcctgatcagcgacttctatcctggcgccgtgaccgtggcctggaaggccgatagctctcctgtgaaggccggcgtggaaaccaccacccctagcaagcagagcaacaacaaatacgccgccagcagctacctgagcctgacccccgagcagtggaagtcccacagatcctacagctgccaagtgacccacgagggcagcaccgtggaaaagacagtggcccctaccgagtgcagc

SEQ ID NO: 127 - protein, 122 aa, Artificial Sequence
  VH TPP-9251
EVQLLESGGGLVQPGGSLRLSCAASGFTFSNYGIHWVRQAPGKGLEWVSAIGTGGDTYYADSVTGRFTISRDNSKNTLYLQMNSLRAEDTAVYYCARDRGYYDILTGLYDYWGQGTLVTVSS

SEQ ID NO: 128 - protein, 5 aa, Artificial Sequence
  HCDR1 TPP-9251
NYGIH

SEQ ID NO: 129 - protein, 16 aa, Artificial Sequence
  HCDR2 TPP-9251
AIGTGGDTYYADSVTG

SEQ ID NO: 130 - protein, 14 aa, Artificial Sequence
  HCDR3 TPP-9251
DRGYYDILTGLYDY

SEQ ID NO: 131 - protein, 109 aa, Artificial Sequence; identical to SEQ ID NO: 117
  VL TPP-9251

SEQ ID NO: 132 - protein, 13 aa, Artificial Sequence
  LCDR1 TPP-9251
SGSSSNIGNNYVN

SEQ ID NO: 133 - protein, 7 aa, Artificial Sequence
  LCDR2 TPP-9251
GNYHRPS

SEQ ID NO: 134 - protein, 10 aa, Artificial Sequence
  LCDR3 TPP-9251
SSYAGSNNWV

SEQ ID NO: 135 - DNA, 366 bp, Artificial Sequence
  VH TPP-9251
gaagtgcagctgctggaaagcggcggaggcctggtgcagcctggcggatctctgagactgagctgtgccgccagcggcttcaccttcagcaactacggcatccactgggtgcgccaggcccctggaaaaggcctggaatgggtgtccgccatcggcacaggcggcgatacctactacgccgatagcgtgaccggcagattcaccatcagccgggacaacagcaagaacaccctgtacctgcagatgaacagcctgcgggccgaggacaccgccgtgtactactgcgctagagacagaggctactacgacatcctgaccggcctgtacgactactggggccagggaacactcgtgaccgtgtcctca

SEQ ID NO: 136 - DNA, 327 bp, Artificial Sequence; identical to SEQ ID NO: 122
  VL TPP-9251

SEQ ID NO: 137 - protein, 451 aa, Artificial Sequence
  Heavy Chain TPP-9251
EVQLLESGGGLVQPGGSLRLSCAASGFTFSNYGIHWVRQAPGKGLEWVSAIGTGGDTYYADSVTGRFTISRDNSKNTLYLQMNSLRAEDTAVYYCARDRGYYDILTGLYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 138 - protein, 215 aa, Artificial Sequence; identical to SEQ ID NO: 124
  Light Chain TPP-9251

SEQ ID NO: 139 - DNA, 1353 bp, Artificial Sequence
  Heavy Chain TPP-9251
gaagtgcagctgctggaaagcggcggaggcctggtgcagcctggcggatctctgagactgagctgtgccgccagcggcttcaccttcagcaactacggcatccactgggtgcgccaggcccctggaaaaggcctggaatgggtgtccgccatcggcacaggcggcgatacctactacgccgatagcgtgaccggcagattcaccatcagccgggacaacagcaagaacaccctgtacctgcagatgaacagcctgcgggccgaggacaccgccgtgtactactgcgctagagacagaggctactacgacatcctgaccggcctgtacgactactggggccagggaacactcgtgaccgtgtcctcagccagcaccaagggccccagcgtgttccctctggcccctagcagcaagagcacatctggcggaacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagctctctgggcacccagacctacatctgcaacgtgaaccacaagcccagcaacaccaaggtggacaagaaggtggaacccaagagctgcgacaagacccacacctgtcccccttgtcctgcccccgaactgctgggaggcccttccgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagcctagagaggaacagtacaacagcacctaccgggtggtgtccgtgctgacagtgctgcaccaggactggctgaacggcaaagagtacaagtgcaaggtgtccaacaaggccctgcctgcccccatcgagaaaaccatcagcaaggccaagggccagccccgcgaaccccaggtgtacacactgcccccaagcagggacgagctgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccctggc

SEQ ID NO: 140 - DNA, 645 bp, Artificial Sequence; identical to SEQ ID NO: 126
  Light Chain TPP-9251

SEQ ID NO: 141 - protein, 122 aa, Artificial Sequence
  VH TPP-9252
EVQLLESGGGLVQPGGSLRLSCAASGFTFSNYGIHWVRQAPGKGLEWVSAIGTGGDTYYADSVQGRFTISRDNSKNTLYLQMNSLRAEDTAVYYCARDRGYYDILTGLYDYWGQGTLVTVSS

SEQ ID NO: 142 - protein, 5 aa, Artificial Sequence
  HCDR1 TPP-9252
NYGIH

SEQ ID NO: 143 - protein, 16 aa, Artificial Sequence
  HCDR2 TPP-9252
AIGTGGDTYYADSVQG

SEQ ID NO: 144 - protein, 14 aa, Artificial Sequence
  HCDR3 TPP-9252
DRGYYDILTGLYDY

SEQ ID NO: 145 - protein, 109 aa, Artificial Sequence; identical to SEQ ID NO: 117
  VL TPP-9252

SEQ ID NO: 146 - protein, 13 aa, Artificial Sequence
  LCDR1 TPP-9252
SGSSSNIGNNYVN

SEQ ID NO: 147 - protein, 7 aa, Artificial Sequence
  LCDR2 TPP-9252
GNYHRPS

SEQ ID NO: 148 - protein, 10 aa, Artificial Sequence
  LCDR3 TPP-9252
SSYAGSNNWV

SEQ ID NO: 149 - DNA, 366 bp, Artificial Sequence
  VH TPP-9252
gaagtgcagctgctggaatctggcggaggattggttcagcctggcggctctctgagactgtcttgtgccgcttctggcttcaccttctccaactacggcatccactgggtccgacaggcccctggaaaaggactggaatgggtgtccgctatcggcaccggcggagatacctactacgccgattctgtgcagggcagattcaccatcagccgggacaactccaagaacaccctgtacctgcagatgaactccctgagagccgaggacaccgccgtgtactactgcgctagagacagaggctactacgacatcctgaccggcctgtacgattactggggccagggaacactggtcaccgttagctca

SEQ ID NO: 150 - DNA, 327 bp, Artificial Sequence
  VL TPP-9252
cagtctgtgttgactcagcctccttccgcttctggcacccctggacagagagtgaccatctcttgctccggctcctcctccaacatcggcaacaactacgtgaactggtatcagcagctgcccggcaccgctcctaaactgctgatctacggcaactaccaccggcctagcggcgtgcccgatagattctctggctctaagtccggcacctctgccagcctggctattactggactgcaggccgaggacgaggccgactactactgttcttcttacgccggctccaacaactgggtgttcggcggaggaacaaagctgaccgttctg

SEQ ID NO: 151 - protein, 451 aa, Artificial Sequence
  Heavy Chain TPP-9252
EVQLLESGGGLVQPGGSLRLSCAASGFTFSNYGIHWVRQAPGKGLEWVSAIGTGGDTYYADSVQGRFTISRDNSKNTLYLQMNSLRAEDTAVYYCARDRGYYDILTGLYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 152 - protein, 215 aa, Artificial Sequence; identical to SEQ ID NO: 124
  Light Chain TPP-9252

SEQ ID NO: 153 - DNA, 1353 bp, Artificial Sequence
  Heavy Chain TPP-9252
gaagtgcagctgctggaatctggcggaggattggttcagcctggcggctctctgagactgtcttgtgccgcttctggcttcaccttctccaactacggcatccactgggtccgacaggcccctggaaaaggactggaatgggtgtccgctatcggcaccggcggagatacctactacgccgattctgtgcagggcagattcaccatcagccgggacaactccaagaacaccctgtacctgcagatgaactccctgagagccgaggacaccgccgtgtactactgcgctagagacagaggctactacgacatcctgaccggcctgtacgattactggggccagggaacactggtcaccgttagctcagcttccaccaagggcccctccgtgttccctctggccccctccagcaagtccacctctggcggcaccgccgctctgggctgcctggtgaaagactacttccccgagcccgtgaccgtgtcctggaactctggcgccctgacctccggcgtgcacacctttccagccgtgctgcagtcctccggcctgtactccctgtcctccgtggtgacagtgccctccagcagcctgggcacccagacctacatctgcaacgtgaaccacaagccctccaacaccaaggtggacaagaaggtggaacccaagtcctgcgacaagacccacacctgtcccccctgccctgcccctgaactgctgggcggaccttccgtgttcctgttccccccaaagcccaaggacaccctgatgatctcccggacccccgaagtgacctgcgtggtggtggacgtgtcccacgaggaccctgaagtgaagttcaattggtacgtggacggcgtggaagtgcacaacgccaagaccaagcccagagaggaacagtacaactccacctaccgggtggtgtccgtgctgaccgtgctgcaccaggactggctgaacggcaaagagtacaagtgcaaggtctccaacaaggccctgcctgctcccatcgaaaagaccatctccaaggccaagggccagccccgcgagcctcaggtctacaccctgcctcccagccgggacgagctgaccaagaaccaggtgtccctgacctgtctggtgaaaggcttctatccctccgacattgccgtggaatgggagtccaacggccagcccgagaacaactacaagaccaccccccctgtgctggactccgacggctcattcttcctgtactccaagctgaccgtggacaagtcccggtggcagcagggcaacgtgttctcctgctccgtgatgcacgaggccctgcacaaccactacacccagaagtccctgtccctgagccccggc

SEQ ID NO: 154 - DNA, 645 bp, Artificial Sequence
  Light Chain TPP-9252
cagtctgtgttgactcagcctccttccgcttctggcacccctggacagagagtgaccatctcttgctccggctcctcctccaacatcggcaacaactacgtgaactggtatcagcagctgcccggcaccgctcctaaactgctgatctacggcaactaccaccggcctagcggcgtgcccgatagattctctggctctaagtccggcacctctgccagcctggctattactggactgcaggccgaggacgaggccgactactactgttcttcttacgccggctccaacaactgggtgttcggcggaggaacaaagctgaccgttctgggccagcctaaggccgctccttccgtgactctgttccctccatcctccgaggaactgcaggctaacaaggctaccctcgtgtgcctgatctccgacttttaccctggcgctgtgaccgtggcctggaaggctgatagttctcctgtgaaggccggcgtggaaaccaccacaccttccaagcagtccaacaacaaatacgccgcctcctcctacctgtctctgacccctgaacagtggaagtcccaccggtcctacagctgccaagtgacccatgagggctccaccgtggaaaagaccgtggctcctaccgagtgctct

SEQ ID NO: 155 - protein, 122 aa, Artificial Sequence
  VH TPP-9258
EVQLLESGGGLVQPGGSLRLSCAASGFTFSSYGMHWVRQAPGKGLEWVSAIGTGGDTYYADSVQGRFTISRDNSKNTLYLQMNSLRAEDTAVYYCARDRGYYDILTGLYDYWGQGTLVTVSS

SEQ ID NO: 156 - protein, 5 aa, Artificial Sequence
  HCDR1 TPP-9258
SYGMH

SEQ ID NO: 157 - protein, 16 aa, Artificial Sequence
  HCDR2 TPP-9258
AIGTGGDTYYADSVQG

SEQ ID NO: 158 - protein, 14 aa, Artificial Sequence
  HCDR3 TPP-9258
DRGYYDILTGLYDY

SEQ ID NO: 159 - protein, 109 aa, Artificial Sequence; identical to SEQ ID NO: 117
  VL TPP-9258

SEQ ID NO: 160 - protein, 13 aa, Artificial Sequence
  LCDR1 TPP-9258
SGSSSNIGNNYVN

SEQ ID NO: 161 - protein, 7 aa, Artificial Sequence
  LCDR2 TPP-9258
GNYHRPS

SEQ ID NO: 162 - protein, 10 aa, Artificial Sequence
  LCDR3 TPP-9258
SSYAGSNNWV

SEQ ID NO: 163 - DNA, 366 bp, Artificial Sequence
  VH TPP-9258
gaagtgcagctgctggaaagcggcggaggcctggtgcagcctggcggatctctgagactgagctgtgccgccagcggcttcaccttcagcagctacggaatgcactgggtgcgccaggcccctggcaaaggactggaatgggtgtccgccatcggcacaggcggcgatacctactacgccgatagcgtgcagggccggttcaccatctcccgggacaacagcaagaacaccctgtacctgcagatgaacagcctgcgggccgaggacaccgccgtgtactactgcgctagagacagaggctactacgacatcctgaccggcctgtacgactactggggccagggaacactcgtgaccgtgtcctca

SEQ ID NO: 164 - DNA, 327 bp, Artificial Sequence; identical to SEQ ID NO: 122
  VL TPP-9258

SEQ ID NO: 165 - protein, 451 aa, Artificial Sequence
  Heavy Chain TPP-9258
EVQLLESGGGLVQPGGSLRLSCAASGFTFSSYGMHWVRQAPGKGLEWVSAIGTGGDTYYADSVQGRFTISRDNSKNTLYLQMNSLRAEDTAVYYCARDRGYYDILTGLYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 166 - protein, 215 aa, Artificial Sequence; identical to SEQ ID NO: 124
  Light Chain TPP-9258

SEQ ID NO: 167 - DNA, 1353 bp, Artificial Sequence
  Heavy Chain TPP-9258
gaagtgcagctgctggaaagcggcggaggcctggtgcagcctggcggatctctgagactgagctgtgccgccagcggcttcaccttcagcagctacggaatgcactgggtgcgccaggcccctggcaaaggactggaatgggtgtccgccatcggcacaggcggcgatacctactacgccgatagcgtgcagggccggttcaccatctcccgggacaacagcaagaacaccctgtacctgcagatgaacagcctgcgggccgaggacaccgccgtgtactactgcgctagagacagaggctactacgacatcctgaccggcctgtacgactactggggccagggaacactcgtgaccgtgtcctcagccagcaccaagggccccagcgtgttccctctggcccctagcagcaagagcacatctggcggaacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagctctctgggcacccagacctacatctgcaacgtgaaccacaagcccagcaacaccaaggtggacaagaaggtggaacccaagagctgcgacaagacccacacctgtcccccttgtcctgcccccgaactgctgggaggcccttccgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagcctagagaggaacagtacaacagcacctaccgggtggtgtccgtgctgacagtgctgcaccaggactggctgaacggcaaagagtacaagtgcaaggtgtccaacaaggccctgcctgcccccatcgagaaaaccatcagcaaggccaagggccagccccgcgaaccccaggtgtacacactgcccccaagcagggacgagctgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccctggc

SEQ ID NO: 168 - DNA, 645 bp, Artificial Sequence; identical to SEQ ID NO: 126
  Light Chain TPP-9258

SEQ ID NO: 169 - protein, 122 aa, Artificial Sequence; identical to SEQ ID NO: 141
  VH TPP-10089

SEQ ID NO: 170 - protein, 5 aa, Artificial Sequence
  HCDR1 TPP-10089
NYGIH

SEQ ID NO: 171 - protein, 16 aa, Artificial Sequence
  HCDR2 TPP-10089
AIGTGGDTYYADSVQG

SEQ ID NO: 172 - protein, 14 aa, Artificial Sequence
  HCDR3 TPP-10089
DRGYYDILTGLYDY

SEQ ID NO: 173 - protein, 109 aa, Artificial Sequence; identical to SEQ ID NO: 117
  VL TPP-10089

SEQ ID NO: 174 - protein, 13 aa, Artificial Sequence
  LCDR1 TPP-10089
SGSSSNIGNNYVN

SEQ ID NO: 175 - protein, 7 aa, Artificial Sequence
  LCDR2 TPP-10089
GNYHRPS

SEQ ID NO: 176 - protein, 10 aa, Artificial Sequence
  LCDR3 TPP-10089
SSYAGSNNWV

SEQ ID NO: 177 - DNA, 366 bp, Artificial Sequence
  VH TPP-10089
gaagtgcagctgctggaaagcggcggaggcctggtgcagcctggcggatctctgagactgagctgtgccgccagcggcttcaccttcagcaactacggcatccactgggtgcgccaggcccctggaaaaggcctggaatgggtgtccgccatcggcacaggcggcgatacctactacgccgatagcgtgcagggccggttcaccatcagccgggacaacagcaagaacaccctgtacctgcagatgaacagcctgcgggccgaggacaccgccgtgtactactgcgctagagacagaggctactacgacatcctgaccggcctgtacgactactggggccagggaacactcgtgaccgtgtcctca

SEQ ID NO: 178 - DNA, 327 bp, Artificial Sequence; identical to SEQ ID NO: 122
  VL TPP-10089

SEQ ID NO: 179 - protein, 225 aa, Artificial Sequence
  Heavy Chain TPP-10089
EVQLLESGGGLVQPGGSLRLSCAASGFTFSNYGIHWVRQAPGKGLEWVSAIGTGGDTYYADSVQGRFTISRDNSKNTLYLQMNSLRAEDTAVYYCARDRGYYDILTGLYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 180 - protein, 215 aa, Artificial Sequence; identical to SEQ ID NO: 124
  Light Chain TPP-10089

SEQ ID NO: 181 - DNA, 675 bp, Artificial Sequence
  Heavy Chain TPP-10089
gaagtgcagctgctggaaagcggcggaggcctggtgcagcctggcggatctctgagactgagctgtgccgccagcggcttcaccttcagcaactacggcatccactgggtgcgccaggcccctggaaaaggcctggaatgggtgtccgccatcggcacaggcggcgatacctactacgccgatagcgtgcagggccggttcaccatcagccgggacaacagcaagaacaccctgtacctgcagatgaacagcctgcgggccgaggacaccgccgtgtactactgcgctagagacagaggctactacgacatcctgaccggcctgtacgactactggggccagggaacactcgtgaccgtgtcctcagcctccaccaagggcccatcggtgttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgt

SEQ ID NO: 182 - DNA, 645 bp, Artificial Sequence; identical to SEQ ID NO: 126
  Light Chain TPP-10089

SEQ ID NO: 183 - protein, 122 aa, Artificial Sequence; identical to SEQ ID NO: 141
  VH TPP-20816

SEQ ID NO: 184 - protein, 5 aa, Artificial Sequence
  HCDR1 TPP-20816
NYGIH

SEQ ID NO: 185 - protein, 16 aa, Artificial Sequence
  HCDR2 TPP-20816
AIGTGGDTYYADSVQG

SEQ ID NO: 186 - protein, 14 aa, Artificial Sequence
  HCDR3 TPP-20816
DRGYYDILTGLYDY

SEQ ID NO: 187 - protein, 109 aa, Artificial Sequence; identical to SEQ ID NO: 117
  VL TPP-20816

SEQ ID NO: 188 - protein, 13 aa, Artificial Sequence
  LCDR1 TPP-20816
SGSSSNIGNNYVN

SEQ ID NO: 189 - protein, 7 aa, Artificial Sequence
  LCDR2 TPP-20816
GNYHRPS

SEQ ID NO: 190 - protein, 10 aa, Artificial Sequence
  LCDR3 TPP-20816
SSYAGSNNWV

SEQ ID NO: 191 - protein, 451 aa, Artificial Sequence
  Heavy Chain 1 TPP-20816
EVQLLESGGGLVQPGGSLRLSCAASGFTFSNYGIHWVRQAPGKGLEWVSAIGTGGDTYYADSVQGRFTISRDNSKNTLYLQMNSLRAEDTAVYYCARDRGYYDILTGLYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 192 - protein, 215 aa, Artificial Sequence; identical to SEQ ID NO: 124
  Light Chain TPP-20816

SEQ ID NO: 193 - protein, 226 aa, Artificial Sequence
  Heavy Chain 2 TPP-20816
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 194 - DNA, 366 bp, Artificial Sequence; identical to SEQ ID NO: 149
  VH TPP-20816

SEQ ID NO: 195 - DNA, 327 bp, Artificial Sequence; identical to SEQ ID NO: 150
  VL TPP-20816

SEQ ID NO: 196 - DNA, 1353 bp, Artificial Sequence
  Heavy Chain 1 TPP-20816
gaagtgcagctgctggaaagcggcggcggcctggtgcagccgggcggcagcctgcgcctgagctgcgcggcgagcggctttacctttagcaactatggcattcattgggtgcgccaggcgccgggcaaaggcctggaatgggtgagcgcgattggcaccggcggcgatacctattatgcggatagcgtgcagggccgctttaccattagccgcgataacagcaaaaacaccctgtatctgcagatgaacagcctgcgcgcggaagataccgcggtgtattattgcgcgcgcgatcgcggctattatgatattctgaccggcctgtatgattattggggccagggcaccctggtgaccgtgagcagcgcgagcaccaaaggcccgagcgtgtttccgctggcgccgagcagcaaaagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatatttgcaacgtgaaccataaaccgagcaacaccaaagtggataaaaaagtggaaccgaaaagctgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgtgccgcgatgaactgaccaaaaaccaggtgagcctgtggtgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggc

SEQ ID NO: 197 - DNA, 645 bp, Artificial Sequence
  Light Chain TPP-20816
cagagcgtgctgacccagccgccgagcgcgagcggcaccccgggccagcgcgtgaccattagctgcagcggcagcagcagcaacattggcaacaactatgtgaactggtatcagcagctgccgggcaccgcgccgaaactgctgatttatggcaactatcatcgcccgagcggcgtgccggatcgctttagcggcagcaaaagcggcaccagcgcgagcctggcgattaccggcctgcaggcggaagatgaagcggattattattgcagcagctatgcgggcagcaacaactgggtgtttggcggcggcaccaaactgaccgtgctgggccagccgaaagcggcgccgagcgtgaccctgtttccgccgagcagcgaagaactgcaggcgaacaaagcgaccctggtgtgcctgattagcgatttttatccgggcgcggtgaccgtggcgtggaaagcggatagcagcccggtgaaagcgggcgtggaaaccaccaccccgagcaaacagagcaacaacaaatatgcggcgagcagctatctgagcctgaccccggaacagtggaaaagccatcgcagctatagctgccaggtgacccatgaaggcagcaccgtggaaaaaaccgtggcgccgaccgaatgcagc

SEQ ID NO: 198 - DNA, 678 bp, Artificial Sequence
  Heavy Chain 2 TPP-20816
gataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtgcaccctgccgccgagccgcgatgaactgaccaaaaaccaggtgagcctgagctgcgcggtgaaaggcttttatccgagcgatattgcggtggaatgggaaagcaacggccagccggaaaacaactataaaaccaccccgccggtgctggatagcgatggcagcttttttctggtgagcaaactgaccgtggataaaagccgctggcagcagggcaacgtgtttagctgcagcgtgatgcatgaagcgctgcataaccattatacccagaaaagcctgagcctgagcccgggc

(19) United States
(12) Patent Application Publication
(10) Pub. No.: US 2021/0395390 A1
(43) Pub. Date: Dec. 23, 2021

(54) REVERSAL AGENTS FOR NEUTRALIZING THE THERAPEUTIC ACTIVITY OF ANTI-FXIA ANTIBODIES

(71) Applicants: Bayer Aktiengesellschaft, Leverkusen (DE); Bayer Pharma Aktiengesellschaft, Berlin (DE)

(72) Inventors: Andreas WILMEN, Köln (DE); Ernst WEBER, Langenfeld (DE)

(73) Assignees: Bayer Aktiengesellschaft, Leverkusen (DE); Bayer Pharma Aktiengesellschaft, Berlin (DE)

(21) Appl. No.: 17/283,490

(22) PCT Filed: Oct. 31, 2019
(86) PCT No.: PCT/EP2019/079802

§ 371 Date: Apr. 7, 2021

(30) Foreign Application Priority Data
Oct. 31, 2018 (EP) 18203700.2

(51) Int. Cl. C07K 16/36

(52) U.S. Cl. CPC C07K 16/36; C07K 2317/21; C07K 2317/92; C07K 2317/55; C07K 2317/76; C07K 2317/35

(57) ABSTRACT

The present invention relates to reversal agents, which specifically bind to the anti-FXIa antibody 076D-M007-H04-CDRL3-N110D as described in WO2013/167669, and neutralize the therapeutic activity of this anti-FXIa antibody, as well as to compositions comprising these reversal agents. Methods of obtaining the antibodies or antigen-binding fragments thereof (such as Fab fragments) and nucleic acids encoding the same, are also provided. Furthermore, the invention relates to methods of use of these reversal agents, such as methods for neutralizing the therapeutic activity of the anti-FXIa antibody 076D-M007-H04-CDRL3-N110D, and to related methods as essential part of a general bleeding management.

### INTRODUCTION

In the course of the last 5 to 10 years, several so called DOAC (Direct Oral Anticoagulants) have been approved by health care authorities for treatment and/or prophylaxis of thromboembolism. These compounds are either directed against thrombin (Dabigatran) or they directly inhibit the coagulation factor Xa (e.g. Rivaroxaban, Apixaban).

Although, these compounds have found their way into clinics, the lack of specific reversal agents is often discussed critically as a major disadvantage of the use of these new anticoagulants.

In 2015, the Cardiac Safety Research Consortium (CSRC), published together with the FDA a white paper (Sarich et al. (2015) Novel oral anticoagulants and reversal agents: Considerations for clinical development. Am Heart J. 169:751-757) where the general need of reversal agents for new anticoagulants is discussed in detail. Although the frequency of need for such agents was considered as extremely low, the CSRC suggested that their availability could improve provider and patient confidence in DOAC use and promote an increase in the appropriate use of anticoagulant therapy.

Recently, some progress has been made with respect to the development of a specific reversal agent for the thrombin inhibitor Dabigatran and for some other FXa inhibitors.

For Dabigatran, a humanized monoclonal antibody fragment has been approved by health care authorities that binds with high affinity to free and thrombin-bound compound, resulting in an almost irreversible bound of the antibody fragment—Dabigatran complex and thereby neutralizing Dabigatran's anticoagulant activity (Reilly et al. (2016) Idarucizumab, a Specific Reversal Agent for Dabigatran: Mode of Action, Pharmacokinetics and Pharmacodynamics, and Safety and Efficacy in Phase 1 Subjects. Am J Med. 129(11S): S64-S72).

In contrast to this, for low molecular weight FXa inhibitors, Andexanet alfa has been designed that specifically reverses the effects of both direct and indirect FXa inhibitors. Andexanet is a recombinant, modified human FXa decoy protein that binds FXa inhibitors but does not have intrinsic catalytic activity (Lu et al. (2013) A specific antidote for reversal of anticoagulation by direct and indirect inhibitors of coagulation FXa. Nat Med 19:446-451; Ghadimi et al. (2016) Andexanet alfa for the reversal of Factor Xa inhibitor related anticoagulation. Expert Rev Hematol 9:115-122).

Specific reversal agents for anti-FXI/FXIa-antibody NOV1401 and anti-FXIa antibody DEF have been described elsewhere (WO2017203450 and WO2017015619).

The fully human monoclonal antibody 076D-M007-H04-CDRL3-N110D as described in WO2013/167669 is a specific inhibitor of the coagulation factor XIa (FXIa) activity leading to a strong and long-lasting antithrombotic activity. Although FXIa is a promising drug target for the development of effective anticoagulants with limited bleeding complications, there is a need for the generation of a specific reversal agent directed against a long-lasting anticoagulant as anti-FXIa-antibody 076D-M007-H04-CDRL3-N110D.

With the anti-076D-M007-H04-CDRL3-N110D monoclonal antibodies, such as full-length antibodies or monovalent antibodies, and antigen-binding fragments thereof, such as Fabs, of this invention, reversal agents have been generated which specifically bind to and thereby neutralize the therapeutic activity of the anti-FXIa antibody 076D-M007-H04-CDRL3-N110D. They are useful for reversing the effects of this anti-FXIa antibody and as essential part of a general bleeding management.

### BRIEF SUMMARY OF THE INVENTION

The present disclosure relates to reversal agents that specifically bind to the anti-FXIa antibody 076D-M007-H04-CDRL3-N110D and thereby inhibit the neutralizing activity of this anti-FXIa antibody.

In certain aspects, the disclosure relates to a monoclonal antibody or antigen-binding fragment thereof, that specifically binds to anti-FXIa antibody 076D-M007-H04-CDRL3-N110D, wherein the antibody or antigen binding fragment thereof comprises HCDR1-3 and LCDR1-3 comprising the amino acid sequences of:

a) SEQ ID NOs: 2, 3, 4, 6, 7, and 8, respectively;

b) SEQ ID NOs: 16, 17, 18, 20, 21, and 22, respectively;

c) SEQ ID NOs: 30, 31, 32, 34, 35, and 36, respectively;

d) SEQ ID NOs: 44, 45, 46, 48, 49, and 50, respectively;

e) SEQ ID NOs: 58, 59, 60, 62, 63, and 64, respectively;

f) SEQ ID NOs: 72, 73, 74, 76, 77, and 78, respectively;

g) SEQ ID NOs: 86, 87, 88, 90, 91, and 92, respectively;

h) SEQ ID NOs: 100, 101, 102, 104, 105, and 106, respectively;

i) SEQ ID NOs: 114, 115, 116, 118, 119, and 120, respectively;

j) SEQ ID NOs: 128, 129, 130, 132, 133, and 134, respectively;

k) SEQ ID NOs: 142, 143, 144, 146, 147, and 148, respectively;

l) SEQ ID NOs: 156, 157, 158, 160, 161, and 162, respectively;

m) SEQ ID NOs: 170, 171, 172, 174, 175, and 176, respectively; or

n) SEQ ID NOs: 184, 185, 186, 188, 189, and 190, respectively;

In certain aspects, the disclosure relates to a monoclonal antibody or antigen-binding fragment thereof that specifically binds to anti-FXIa antibody 076D-M007-H04-CDRL3-N110D, wherein the antibody or antigen binding fragment thereof comprises a variable heavy chain (VH) sequence and a variable light chain (VL) sequence comprising the amino acid sequences of:

a) SEQ ID NOs: 1 and 5, respectively;

b) SEQ ID NOs: 15 and 19, respectively;

c) SEQ ID NOs: 29 and 33, respectively;

d) SEQ ID NOs: 43 and 47, respectively;

e) SEQ ID NOs: 57 and 61, respectively;

f) SEQ ID NOs: 71 and 75, respectively;

g) SEQ ID NOs: 85 and 89, respectively;

h) SEQ ID NOs: 99 and 103, respectively;

i) SEQ ID NOs: 113 and 117, respectively;

j) SEQ ID NOs: 127 and 131, respectively;

k) SEQ ID NOs: 141 and 145, respectively;

l) SEQ ID NOs: 155 and 159, respectively;

m) SEQ ID NOs: 169 and 173, respectively; or

n) SEQ ID NOs: 183 and 187, respectively In certain aspects, the disclosure relates to a monoclonal antibody or antigen-binding fragment thereof that specifically binds to anti-FXIa antibody 076D-M007-H04-CDRL3-N110D, wherein the antibody or antigen binding fragment thereof comprises: a heavy chain sequence and a light chain sequence comprising the amino acid sequences of:

a) SEQ ID NOs: 11 and 12, respectively;

b) SEQ ID NOs: 25 and 26, respectively;

c) SEQ ID NOs: 39 and 40, respectively;

d) SEQ ID NOs: 53 and 54, respectively;

e) SEQ ID NOs: 67 and 68, respectively;

f) SEQ ID NOs: 81 and 82, respectively;

g) SEQ ID NOs: 95 and 96, respectively;

h) SEQ ID NOs: 109 and 110, respectively;

i) SEQ ID NOs: 123 and 124, respectively;

j) SEQ ID NOs: 137 and 138, respectively;

k) SEQ ID NOs: 151 and 152, respectively;

l) SEQ ID NOs: 165 and 166, respectively; or

m) SEQ ID NOs: 179 and 180, respectively; In certain aspects, the disclosure relates to a monoclonal monovalent antibody or antigen-binding fragment thereof that specifically binds to anti-FXIa antibody 076D-M007-H04-CDRL3-N110D, wherein the monovalent antibody or antigen binding fragment thereof comprises heavy chain sequences comprising the amino acid sequences of SEQ ID NOs: 191 and 193, respectively and a light chain sequence comprising the amino acid sequence of SEQ ID NO 192.

In some embodiments, the monoclonal antibody or antigen-binding fragment thereof that specifically binds to anti-FXIa antibody 076D-M007-H04-CDRL3-N110D is chimeric, humanized, or human.

In some embodiments, the monoclonal antibody or antigen-binding fragment thereof that specifically binds to anti-FXIa antibody 076D-M007-H04-CDRL3-N110D comprises a human IgG heavy chain constant region.

In some embodiments, the monoclonal antibody or antigen-binding fragment thereof that specifically binds to anti-FXIa antibody 076D-M007-H04-CDRL3-N110D comprises a human IgG1 heavy chain constant region.

In some embodiments, the monoclonal antibody that specifically binds to anti-FXIa antibody 076D-M007-H04-CDRL3-N110D is a full-length antibody.

In some embodiments, the monoclonal antibody that specifically binds to anti-FXIa antibody 076D-M007-H04-CDRL3-N110D is a monovalent antibody.

In some embodiments, the monovalent antibody that specifically binds to anti-FXIa antibody 076D-M007-H04-CDRL3-N110D is a monovalent antibody derived from a full-length antibody.

In some embodiments, the antigen-binding fragment of the monoclonal antibody that specifically binds to anti-FXIa antibody 076D-M007-H04-CDRL3-N110D is a Fab fragment.

In certain preferred aspects, the disclosure relates to a monoclonal antibody or antigen-binding fragment thereof that specifically binds to anti-FXIa antibody 076D-M007-H04-CDRL3-N110D and thereby inhibits the neutralizing activity of this anti-FXIa antibody, wherein the antibody or antigen binding fragment thereof comprises HCDR1-3 and LCDR1-3 comprising the amino acid sequences of:

a) SEQ ID NOs: 72, 73, 74, 76, 77, and 78, respectively;

b) SEQ ID NOs: 86, 87, 88, 90, 91, and 92, respectively;

c) SEQ ID NOs: 114, 115, 116, 118, 119, and 120, respectively;

d) SEQ ID NOs: 128, 129, 130, 132, 133, and 134, respectively;

e) SEQ ID NOs: 142, 143, 144, 146, 147, and 148, respectively;

f) SEQ ID NOs: 156, 157, 158, 160, 161, and 162, respectively;

g) SEQ ID NOs: 170, 171, 172, 174, 175, and 176, respectively; or

h) SEQ ID NOs: 184, 185, 186, 188, 189, and 190, respectively.

In certain preferred aspects, the disclosure relates to a monoclonal antibody or antigen-binding fragment thereof that specifically binds to anti-FXIa antibody 076D-M007-H04-CDRL3-N110D and thereby inhibits the neutralizing activity of this anti-FXIa antibody, wherein the antibody or antigen binding fragment thereof comprises a variable heavy chain (VH) sequence and a variable light chain (VL) sequence comprising the amino acid sequences of:

a) SEQ ID NOs: 71 and 75, respectively;

b) SEQ ID NOs: 85 and 89, respectively;

c) SEQ ID NOs: 113 and 117, respectively;

d) SEQ ID NOs: 127 and 131, respectively;

e) SEQ ID NOs: 141 and 145, respectively;

f) SEQ ID NOs: 155 and 159, respectively;

g) SEQ ID NOs: 169 and 173, respectively; or

h) SEQ ID NOs: 183 and 187, respectively.

In certain preferred aspects, the disclosure relates to a monoclonal antibody or antigen-binding fragment thereof that specifically binds to anti-FXIa antibody 076D-M007-H04-CDRL3-N110D and thereby inhibits the neutralizing activity of this anti-FXIa antibody, wherein the antibody or antigen binding fragment thereof comprises: a heavy chain sequence and a light chain sequence comprising the amino acid sequences of:

a) SEQ ID NOs: 81 and 82, respectively;

b) SEQ ID NOs: 95 and 96, respectively;

c) SEQ ID NOs: 123 and 124, respectively;

d) SEQ ID NOs: 137 and 138, respectively;

e) SEQ ID NOs: 151 and 152, respectively;

f) SEQ ID NOs: 165 and 166, respectively;

g) SEQ ID NOs: 179 and 180, respectively; or

h) SEQ ID NOs: 191 and 192, respectively.

In certain preferred aspects, the disclosure relates to a monovalent antibody or antigen-binding fragment thereof that specifically binds to anti-FXIa antibody 076D-M007-H04-CDRL3-N110D and thereby inhibits the neutralizing activity of this anti-FXIa antibody, wherein the antibody or antigen binding fragment thereof comprises: heavy chain sequences comprising the amino acid sequences of SEQ ID NOs: 191 and 193, respectively and a light chain sequence comprising the amino acid sequence of SEQ ID NO: 192.

In certain especially preferred aspects, the disclosure relates to a monoclonal antibody or antigen-binding fragment thereof that specifically binds to anti-FXIa antibody 076D-M007-H04-CDRL3-N110D and thereby inhibits the neutralizing activity of this anti-FXIa antibody, wherein the antibody or antigen binding fragment thereof comprises HCDR1-3 and LCDR1-3 comprising the amino acid sequences of SEQ ID NOs: 142, 143, 144, 146, 147, and 148, respectively; or of SEQ ID NOs: 170, 171, 172, 174, 175, and 176, respectively or of SEQ ID NOs: 184, 185, 186, 188, 189 and 190, respectively.

In certain preferred aspects, the disclosure relates to a monoclonal antibody or antigen-binding fragment thereof that specifically binds to anti-FXIa antibody 076D-M007-H04-CDRL3-N110D and thereby inhibits the neutralizing activity of this anti-FXIa antibody, wherein the antibody or antigen binding fragment thereof comprises a variable heavy chain (VH) sequence and a variable light chain (VL) sequence comprising the amino acid sequences of SEQ ID NOs: 141 and 145, or SEQ ID NOs: 169 and 173, or SEQ ID NOs: 183 and 187, respectively.

In certain especially preferred aspects, the disclosure relates to a monoclonal antibody that specifically binds to anti-FXIa antibody 076D-M007-H04-CDRL3-N110D and thereby inhibits the neutralizing activity of this anti-FXIa antibody, wherein the antibody or antigen binding fragment thereof comprises the heavy chain sequence of SEQ ID NOs: 151 and the light chain sequence of SEQ ID NO: 152.

In further especially preferred aspects, the disclosure relates to a monovalent antibody that specifically binds to anti-FXIa antibody 076D-M007-H04-CDRL3-N110D and thereby inhibits the neutralizing activity of this anti-FXIa antibody, wherein the monovalent antibody or antigen binding fragment thereof comprises the heavy chain sequences of SEQ ID NOs: 191 and 193, respectively and the light chain sequence of SEQ ID NO: 192.

In some embodiments, the monoclonal antibody or antigen-binding fragment thereof that specifically binds to anti-FXIa antibody 076D-M007-H04-CDRL3-N110D and thereby inhibits the neutralizing activity of this anti-FXIa antibody is chimeric, humanized, or human.

In some embodiments, the monoclonal antibody or antigen-binding fragment thereof that specifically binds to anti-FXIa antibody 076D-M007-H04-CDRL3-N110D and thereby inhibits the neutralizing activity of this anti-FXIa antibody comprises a human IgG heavy chain constant region.

In some embodiments, the monoclonal antibody or antigen-binding fragment thereof that specifically binds to anti-FXIa antibody 076D-M007-H04-CDRL3-N110D and thereby inhibits the neutralizing activity of this anti-FXIa antibody comprises a human IgG1 heavy chain constant region.

In some embodiments, the monoclonal antibody or antigen-binding fragment thereof that specifically binds to anti-FXIa antibody 076D-M007-H04-CDRL3-N110D and thereby inhibits the neutralizing activity of this anti-FXIa antibody comprises a human IgG4 heavy chain constant region.

In some embodiments, the monoclonal antibody or antigen-binding fragment thereof that specifically binds to anti-FXIa antibody 076D-M007-H04-CDRL3-N110D and thereby inhibits the neutralizing activity of this anti-FXIa antibody comprises a human IgG2 heavy chain constant region.

In some embodiments, the monoclonal antibody that specifically binds to anti-FXIa antibody 076D-M007-H04-CDRL3-N110D and thereby inhibits the neutralizing activity of this anti-FXIa antibody is a monovalent antibody.

In some embodiments, the monovalent antibody that specifically binds to anti-FXIa antibody 076D-M007-H04-CDRL3-N110D and thereby inhibits the neutralizing activity of this anti-FXIa antibody comprises a human IgG1 heavy chain constant region.

In some embodiments, the monovalent antibody that specifically binds to anti-FXIa antibody 076D-M007-H04-CDRL3-N110D and thereby inhibits the neutralizing activity of this anti-FXIa antibody comprises a human IgG2 or IgG4 heavy chain constant region.

In especially preferred embodiments, the disclosure relates to a monovalent antibody that specifically binds to anti-FXIa antibody 076D-M007-H04-CDRL3-N110D and thereby inhibits the neutralizing activity of this anti-FXIa antibody, wherein the monovalent antibody comprises the heavy chain sequences of SEQ ID NOs: 191 and 193 and the light chain sequence of SEQ ID NO: 192.

In some embodiments, the monoclonal antigen-binding fragment that specifically binds to anti-FXIa antibody 076D-M007-H04-CDRL3-N110D and thereby inhibits the neutralizing activity of this anti-FXIa antibody is a Fab fragment.

In further especially preferred aspects, the disclosure relates to a Fab fragment that specifically binds to anti-FXIa antibody 076D-M007-H04-CDRL3-N110D and thereby inhibits the neutralizing activity of this anti-FXIa antibody, wherein the Fab fragment comprises the heavy chain sequence of SEQ ID NOs: 179 and the light chain sequence of SEQ ID NO: 180.

In certain aspects, the disclosure relates to an isolated nucleic acid molecule comprising a nucleotide sequence encoding a monoclonal antibody or an antigen-binding fragment thereof that specifically binds to anti-FXIa antibody 076D-M007-H04-CDRL3-N1101D.

In certain preferred aspects, the disclosure relates to an isolated nucleic acid molecule comprising a nucleotide sequence encoding a monoclonal antibody or an antigen-binding fragment thereof that specifically binds to anti-FXIa antibody 076D-M007-H04-CDRL3-N110D and thereby inhibits the neutralizing activity of this anti-FXIa antibody.

In certain especially preferred aspects, the disclosure relates to an isolated nucleic acid molecule comprising a nucleotide sequence encoding a monoclonal antibody or an antigen-binding fragment thereof that specifically binds to anti-FXIa antibody 076D-M007-H04-CDRL3-N110D and thereby inhibits the neutralizing activity of this anti-FXIa antibody, wherein the nucleic acid molecule comprises the nucleotide sequences of SEQ ID NO: 149 and 150 (TPP-9252) or SEQ ID NO: 177 and SEQ ID NO: 178 (TPP-10089), or SEQ ID NO: 194 and SEQ ID NO: 195 (TPP-20816), respectively.

In a further aspect, the disclosure provides a vector, particularly an expression vector, comprising said nucleic acid molecule. The invention also relates to a host cell comprising said vector or nucleic acid molecule.

In a further aspect, a process for the production of an antibody or antigen-binding fragment thereof as described herein is provided, said process comprising culturing a host cell as defined herein under conditions allowing the expression of said antibody or antigen-binding fragment thereof and optionally recovering the produced antibody or antigen-binding fragment thereof from the culture.

Moreover, the invention relates to a pharmaceutical composition comprising an antibody or antigen-binding fragment thereof, a nucleic acid molecule encoding the amino acid sequences of this antibody or fragment thereof, the vector and/or the host cell as defined herein, and optionally a pharmaceutically acceptable excipient. Said pharmaceutical composition may comprise additional active agents or be administered as part of combination therapy with additional active agents. Compositions also include variants and derivatives of these antibodies or antigen-binding fragments thereof, cell lines producing these antibodies, fragments, variants, and derivatives, isolated nucleic acid molecules encoding the amino acid sequences of these antibodies or antigen-binding fragments thereof.

According to the present invention, the antibody or antigen-binding fragment thereof, the isolated nucleic acid molecule encoding the amino acid sequences of this antibody or fragment thereof, the vector, the host cell and/or the pharmaceutical composition can be used in a method of neutralizing the therapeutic activity of the anti-FXIa antibody 076D-M007-H04-CDRL3-N110D in a subject in need thereof. They are useful as reversal agents for neutralizing the therapeutic activity of this anti-FXIa antibody and for related methods as essential part of a general bleeding management.

Further provided herein is the use of the antibody or antigen-binding fragment according to the present invention as a reversal agent for reversing the effects of anti-FXIa antibody 076D-M007-H04-CDRL3-N110D in blood samples, blood preservations, plasma products, biological samples, or medicinal additives or as a coating on medical devices.

Moreover, the present invention relates to a kit comprising an antibody or antigen-binding fragment thereof, a nucleic acid molecule encoding the amino acid sequences of this antibody or fragment thereof, a vector, a host cell or the pharmaceutical composition as described herein.

### BRIEF DESCRIPTION OF THE FIGURES

FIG. 1 shows binding activities of antibodies of this invention to the anti-FXIa antibody 076D-M007-H04-CDRL3-N110D. Binding curves are shown from standard binding ELISA experiments as described in Example 4. Binding activities were calculated and are expressed as EC50 as log M. Average binding curves from two to three individual experiments are shown.

FIG. 2 shows the catalytic activity of human FXIa. Proteolytic activity of isolated human FXIa was calculated and expressed as EC50 as log M. Average activity curves from three individual experiments are shown.

FIG. 3 a-c show the neutralizing activity of different antibodies binding to anti-FXIa antibody 076D-M007-H04-CDRL3-N110D. Different antibodies binding to anti-FXIa antibody 076D-M007-H04-CDRL3-N110D were tested for blocking the anti-FXIa activity of 076D-M007-H04-CDRL3-N110D as described in Example 5: Activity testing. Despite their high binding activity to 076D-M007-H04-CDRL3-N110D, the majority of the identified anti-076D-M007-H04-CDRL3-N110D antibodies didn't show any neutralizing activity. Only two antibodies, TPP8243 and TPP-8241, showed—with increasing concentrations—a blockade of 076D-M007-H04-CDRL3-N110D, determined by the recovered proteolytic activity of FXIa. The IC50 values are expressed log M values and are listed in Table 3.

FIG. 4 shows the neutralizing activity of TPP-8241 and TPP-8243 (EC50 values in nM) in plasma-based activity assay. TPP-8241 and TPP-8243 were able to restore the 076D-M007-H04-CDRL3-N110D mediated FXIa blockade.

FIG. 5 shows the effect of different doses of TPP-9252 (1.5-5-15 mg/kg i.v. bolus) administered to anesthetized rabbits 15 min after i.v. dosing of 076D-M007-H04-CDRL3-N110D (3 mg/kg) on aPTT (Mean±SEM of 2-3 animals/group). As shown, doses of 5 mg/kg and 15 mg/kg of TPP-9252 were able to reduce aPTT elongation back to baseline level (>90% normalization). This corresponds to a molar excess of 1.7-fold for 5 mg/kg of TPP-9252. Following this dose response curve, a molar excess greater than 2-fold is expected to provide full return to baseline.

FIG. 6 shows the effect of double application of Fab TPP-10089 (2×10 mg/kg, i.v. bolus, time interval 60 min) administered to anesthetized rabbits 15 min after i.v. dosing of 076D-M007-H04-CDRL3-N110D (3 mg/kg) on aPTT (Mean±SEM of 2-3 animals/group). As shown, in contrast to the full-length IgG TPP-9252, the administration of the Fab TPP-10089 only leads to a transient aPTT-normalization.

FIG. 7 depicts the amino acid and nucleic acid sequences of the reversal agents according to the invention.

### DESCRIPTION OF THE INVENTION

The present invention is based on the discovery of novel reversal agents, which specifically bind to the anti-FXIa antibody 076D-M007-H04-CDRL3-N110D as described in WO2013/167669 and neutralize the therapeutic activity of this anti-FXIa antibody. The reversal agents of the invention, which may be human, humanized or chimeric antibodies or antigen-binding fragments thereof, such as Fabs, can be used in many contexts, which are more fully described herein.

Unless defined otherwise, all technical and scientific terms used herein have the meaning commonly understood by one of ordinary skill in the art to which this invention belongs. The following references, however, can provide one of skill in the art to which this invention pertains with a general definition of many of the terms used in this invention, and can be referenced and used so long as such definitions are consistent with the meaning commonly understood in the art. Such references include, but are not limited to, Singleton et al., Dictionary of Microbiology and Molecular Biology (2nd ed. 1994); The Cambridge Dictionary of Science and Technology (Walker ed., 1988); Hale & Marham, The Harper Collins Dictionary of Biology (1991); and Lackie et al., The Dictionary of Cell & Molecular Biology (3d ed. 1999); and Cellular and Molecular Immunology, Eds. Abbas, Lichtman and Pober, 2nd Edition, W.B. Saunders Company. Any additional technical resource available to the person of ordinary skill in the art providing definitions of terms used herein having the meaning commonly understood in the art can be consulted. For the purposes of the present invention, the following terms are further defined. Additional terms are defined elsewhere in the description. As used herein and in the appended claims, the singular forms “a,” and “the” include plural reference unless the context clearly dictates otherwise. Thus, for example, reference to “a gene” is a reference to one or more genes and includes equivalents thereof known to those skilled in the art, and so forth.

The coagulation Factor XI (FXI) is synthesized in the liver and circulates in the plasma as a disulfide bond-linked dimer complexed with High Molecular Weight Kininogen. Each polypeptide chain of this dimer is approximately 80 kD. The zymogen Factor XI is converted into its active form, the coagulation factor Xla (FXla) either via the contact phase of blood coagulation or through Thrombin-mediated activation on the platelet surface. During this activation of factor XI, an internal peptide bond is cleaved in each of the two chains, resulting in the activated factor Xla, a serine protease composed of two heavy and two light chains held together by disulfide bonds. This serine protease FXla converts the coagulation Factor IX into IXa, which subsequently activates coagulation Factor X (Xa). Xa then can mediate coagulation Factor II/Thrombin activation. Defects in this factor lead to Rosenthal syndrome (also known as hemophilia C), a blood coagulation abnormality characterized by prolonged bleeding from injuries, frequent or heavy nosebleeds, traces of blood in the urine, and heavy menstrual bleeding in females. As used herein, “coagulation factor XI,” “factor XI”, or “FXI” refers to any FXI from any mammalian species that expresses the protein. For example, FXI can be human, nonhuman primate (such as baboon), mouse, dog, cat, cow, horse, pig, rabbit, and any other species exhibiting the coagulation factor XI involved in the regulation of blood flow, coagulation, and/or thrombosis.

The cleavage site for the activation of the coagulation factor XI by the coagulation factor Xlla is an internal peptide bond between Arg-369 and lie-370 in each polypeptide chain [Fujikawa K, Chung D W, Hendrickson L E, Davie E W. (1986) Amino acid sequence of human factor XI, a blood coagulation factor with four tandem repeats that are highly homologous with plasma prekallikrein. Biochemistry 25:2417-2424]. Each heavy chain of the coagulation factor Xla (369 amino acids) contains four tandem repeats of 90-91 amino acids called apple domains (designated A1-A4) plus a short connecting peptide [Fujikawa K, Chung D W, Hendrickson L E, Davie E W. (1986) Amino acid sequence of human factor XI, a blood coagulation factor with four tandem repeats that are highly homologous with plasma prekallikrein. Biochemistry 25:2417-2424; Sun M F, Zhao M, Gailani D. (1999). Identification of amino acids in the factor XI apple 3 domain required for activation of factor IX. J Biol Chem. 274:36373-36378]. The light chains of the coagulation factor Xla (each 238 amino acids) contain the catalytic portion of the enzyme with sequences that are typical of the trypsin family of serine proteases [Fujikawa K, Chung D W, Hendrickson L E, Davie E W. (1986) Amino acid sequence of human factor XI, a blood coagulation factor with four tandem repeats that are highly homologous with plasma prekallikrein. Biochemistry 25:2417-2424]. Activated factor Xla triggers the middle phase of the intrinsic pathway of blood coagulation by activating factor IX. As used herein, “coagulation factor XIa,” “factor XIa”, or “FXIa” refers to any FXIa from any mammalian species that expresses the protein. For example, FXIa can be human, nonhuman primate (such as baboon), mouse, dog, cat, cow, horse, pig, rabbit, and any other species exhibiting the coagulation factor XI involved in the regulation of blood flow, coagulation, and/or thrombosis.

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

Amino acids may be referred to herein by their commonly known three letter symbols or by the one-letter symbols recommended by the IUPAC-IUB Biochemical Nomenclature Commission. Nucleotides, likewise, may be referred to by their commonly accepted single-letter codes.

The term “antibody”, as used herein, is intended to refer to immunoglobulin molecules including, but not limited to, full-length antibodies and monovalent antibodies. “Full-length antibodies” are preferably comprised of four polypeptide chains, two heavy (H) chains and two light (L) chains which are typically inter-connected by disulfide bonds. Each heavy chain is comprised of a heavy chain variable region (abbreviated herein as VH) and a heavy chain constant region. The heavy chain constant region can comprise e.g. three domains CH1, CH2 and CH3. Each light chain is comprised of a light chain variable region (abbreviated herein as VL) and a light chain constant region. The light chain constant region is comprised of one domain (CL). The VH and VL regions can be further subdivided into regions of hypervariability, termed complementarity determining regions (CDR), interspersed with regions that are more conserved, termed framework regions (FR). Each VH and VL is typically composed of three CDRs and up to four FRs arranged from amino-terminus to carboxy-terminus e.g. in the following order: FR1, CDR1, FR2, CDR2, FR3, CDR3, FR4. “Monovalent antibodies” as used herein are preferably comprised of three polypeptide chains, two heavy (H) chains and one light (L) chain which are typically inter-connected by disulfide bonds. One heavy chain is comprised of a heavy chain variable region (abbreviated herein as VH) and a heavy chain constant region. The heavy chain constant region can comprise e.g. three domains CH1, CH2 and CH3. The other heavy chain is comprised of a heavy chain constant region only. The light chain is comprised of a light chain variable region (abbreviated herein as VL) and a light chain constant region. The light chain constant region is comprised of one domain (CL). The VH and VL regions can be further subdivided into regions of hypervariability, termed complementarity determining regions (CDR), interspersed with regions that are more conserved, termed framework regions (FR). Each VH and VL is typically composed of three CDRs and up to four FRs arranged from amino-terminus to carboxy-terminus e.g. in the following order: FR1, CDR1, FR2, CDR2, FR3, CDR3, FR4.

As used herein, the term “Complementarity Determining Regions” (CDRs; e.g., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (e.g. about residues 24-34 (L1), 50-56 (L2) and 89-97 (L3) in the light chain variable domain and 31-35 (H1), 50-65 (H2) and 95-102 (H3) in the heavy chain variable domain; (Kabat et al., Sequences of Proteins of Immunological Interest, 5th Ed. Public Health Service, National Institutes of Health, Bethesda, Md. (1991)) and/or those residues from a “hypervariable loop” (e.g. about residues 26-32 (L1), 50-52 (L2) and 91-96 (L3) in the light chain variable domain and 26-32 (H1), 53-55 (H2) and 96-101 (H3) in the heavy chain variable domain (Chothia and Lesk; J Mol Biol 196: 901-917 (1987)). In some instances, a complementarity determining region can include amino acids from both a CDR region defined according to Kabat and a hypervariable loop.

Depending on the amino acid sequence of the constant domain of their heavy chains, intact antibodies can be assigned to different “classes”. There are five major classes of intact antibodies: IgA, IgD, IgE, IgG, and IgM, and several of these maybe further divided into “subclasses” (isotypes), e.g., IgG1, IgG2, IgG3, IgG4, IgA1, and IgA2. A preferred class of immunoglobulins for use in the present invention is IgG.

The heavy-chain constant domains that correspond to the different classes of antibodies are called [alpha], [delta], [epsilon], [gamma], and [mu], respectively. The subunit structures and three-dimensional configurations of different classes of immunoglobulins are well known. As used herein antibodies are conventionally known antibodies and functional fragments thereof.

A “functional fragment” or “antigen-binding antibody fragment” of an antibody/immunoglobulin hereby is defined as a fragment of an antibody/immunoglobulin (e.g., a variable region of an IgG) that retains the antigen-binding region. An “antigen-binding region” of an antibody typically is found in one or more hyper variable region(s) of an antibody, e.g., the CDR1, -2, and/or -3 regions; however, the variable “framework” regions can also play an important role in antigen binding, such as by providing a scaffold for the CDRs.

“Functional fragments”, “antigen-binding antibody fragments”, or “antibody fragments” of the invention include but are not limited to Fab, Fab′, Fab′-SH, F(ab′)₂, and Fv fragments; diabodies; single domain antibodies (DAbs), linear antibodies; single-chain antibody molecules (scFv); and multi-specific, such as bi- and tri-specific, antibodies formed from antibody fragments (C. A. K Borrebaeck, editor (1995) Antibody Engineering (Breakthroughs in Molecular Biology), Oxford University Press; R. Kontermann & S. Duebel, editors (2001) Antibody Engineering (Springer Laboratory Manual), Springer Verlag). An antibody other than a “multi-specific” or “multi-functional” antibody is understood to have each of its binding sites identical. The F(ab′)₂ or Fab may be engineered to minimize or completely remove the intermolecular disulfide interactions that occur between the CH1 and CL domains.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

Variants of the antibodies or antigen-binding antibody fragments contemplated in the invention are molecules in which the binding activity of the antibody or antigen-binding antibody fragment is maintained.

“Binding proteins” contemplated in the invention are for example antibody mimetics, such as Affibodies, Adnectins, Anticalins, DARPins, Avimers, Nanobodies (reviewed by Gebauer M. et al., Curr. Opinion in Chem. Biol. 2009; 13:245-255; Nuttall S. D. et al., Curr. Opinion in Pharmacology 2008; 8:608-617).

A “human” antibody or antigen-binding fragment thereof is hereby defined as one that is not chimeric (e.g., not “humanized”) and not from (either in whole or in part) a non-human species. A human antibody or antigen-binding fragment thereof can be derived from a human or can be a synthetic human antibody. A “synthetic human antibody” is defined herein as an antibody having a sequence derived, in whole or in part, in silico from synthetic sequences that are based on the analysis of known human antibody sequences. In silico design of a human antibody sequence or fragment thereof can be achieved, for example, by analyzing a database of human antibody or antibody fragment sequences and devising a polypeptide sequence utilizing the data obtained there from. Another example of a human antibody or antigen-binding fragment thereof is one that is encoded by a nucleic acid isolated from a library of antibody sequences of human origin (e.g., such library being based on antibodies taken from a human natural source). Examples of human antibodies include antibodies as described in Söderlind et al., Nature Biotech. 2000, 18:853-856.

A “humanized antibody” or humanized antigen-binding fragment thereof is defined herein as one that is (i) derived from a non-human source (e.g., a transgenic mouse which bears a heterologous immune system), which antibody is based on a human germline sequence; (ii) where amino acids of the framework regions of a non-human antibody are partially exchanged to human amino acid sequences by genetic engineering or (iii) CDR-grafted, wherein the CDRs of the variable domain are from a non-human origin, while one or more frameworks of the variable domain are of human origin and the constant domain (if any) is of human origin.

A “chimeric antibody” or antigen-binding fragment thereof is defined herein as one, wherein the variable domains are derived from a non-human origin and some or all constant domains are derived from a human origin.

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the term “monoclonal” indicates the character of the antibody as not being a mixture of discrete antibodies. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins. The term “monoclonal” is not to be construed as to require production of the antibody by any particular method. The term monoclonal antibody specifically includes chimeric, humanized and human antibodies.

An “isolated” antibody is one that has been identified and separated from a component of the cell that expressed it. Contaminant components of the cell are materials that would interfere with diagnostic or therapeutic uses of the antibody, and may include enzymes, hormones, and other proteinaceous or non-proteinaceous solutes.

An “isolated” nucleic acid is one that has been identified and separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

As used herein, an antibody “binds specifically to”, is “specific to/for” or “specifically recognizes” an antigen of interest, e.g. a tumor-associated polypeptide antigen target or an antigen-binding polypeptide target (as e.g. an antigen-binding antibody), is one that binds the antigen-target with sufficient affinity such that the antibody is useful as a therapeutic agent in targeting a cell or tissue expressing the antigen or one that binds an antigen-binding polypeptide target with sufficient affinity such that the antibody is useful as a reversal agent to neutralize the therapeutic activity of this antigen-binding polypeptide (e.g. an antigen-binding antibody) and does not significantly cross-react with other proteins or does not significantly cross-react with proteins other than orthologs and variants (e.g. mutant forms, splice variants, or proteolytically truncated forms) of the aforementioned target. The term “specifically recognizes” or “binds specifically to” or is “specific to/for” a particular polypeptide or an epitope on a particular polypeptide target as used herein can be exhibited, for example, by an antibody, or antigen-binding fragment thereof, having a monovalent K_{D} for the antigen of less than about 10⁻⁴ M, alternatively less than about 10⁻⁵ M, alternatively less than about 10⁻⁶ M, alternatively less than about 10⁻⁷ M, alternatively less than about 10⁻⁸ M, alternatively less than about 10⁻⁹ M, alternatively less than about 10⁻¹⁰ M, alternatively less than about 10⁻¹¹ M, alternatively less than about 10⁻¹² M, or less. An antibody “binds specifically to,” is “specific to/for” or “specifically recognizes” an antigen if such antibody is able to discriminate between such antigen and one or more reference antigen(s). In its most general form, “specific binding”, “binds specifically to”, is “specific to/for” or “specifically recognizes” is referring to the ability of the antibody to discriminate between the antigen of interest and an unrelated antigen, as determined, for example, in accordance with one of the following methods. Such methods comprise, but are not limited to, surface plasmon resonance (SPR), Western blots, ELISA-, RIA-, ECL-, IRMA-tests and peptide scans. For example, a standard ELISA assay can be carried out. The scoring may be carried out by standard color development (e.g. secondary antibody with horseradish peroxidase and tetramethyl benzidine with hydrogen peroxide). The reaction in certain wells is scored by the optical density, for example, at 450 nm. Typical background (=negative reaction) may be 0.1 OD; typical positive reaction may be 1 OD. This means the difference positive/negative is more than 5-fold, 10-fold, 50-fold, and preferably more than 100-fold. Typically, determination of binding specificity is performed by using not a single reference antigen, but a set of about three to five unrelated antigens, such as milk powder, BSA, transferrin or the like.

“Binding affinity” or “affinity” refers to the strength of the total sum of non-covalent interactions between a single binding site of a molecule and its binding partner. Unless indicated otherwise, as used herein, “binding affinity” refers to intrinsic binding affinity which reflects a 1:1 interaction between members of a binding pair (e.g. an antibody and an antigen). The dissociation constant “K_{D}” is commonly used to describe the affinity between a molecule (such as an antibody) and its binding partner (such as an antigen) i.e. how tightly a ligand binds to a particular protein. Ligand-protein affinities are influenced by non-covalent intermolecular interactions between the two molecules. Affinity can be measured by common methods known in the art, including those described herein. In one embodiment, the “K_{D}” or “K_{D} value” according to this invention is measured by using surface plasmon resonance assays using suitable devices including but not limited to Biacore instruments like Biacore T100, Biacore T200, Biacore 2000, Biacore 4000, a Biacore 3000 (GE Healthcare Biacore, Inc.), or a ProteOn XPR36 instrument (Bio-Rad Laboratories, Inc.).

“Antibody-dependent cell-mediated cytotoxicity” or “ADCC” refers to a form of cytotoxicity in which secreted Ig bound onto Fc gamma receptors (FcγRs) present on certain cytotoxic cells (e.g. NK cells, neutrophils, and macrophages) enable these cytotoxic effector cells to bind specifically to an antigen-bearing target cell and subsequently kill the target cell e.g. with cytotoxins. To assess ADCC activity of an antibody of interest, an in vitro ADCC assay, such as that described in U.S. Pat. No. 5,500,362 or 5,821,337 or 6,737,056 (Presta), may be performed. Useful effector cells for such assays include PBMC and NK cells.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass), which are bound to their cognate antigen. To assess complement activation, a CDC assay, e.g., as described in Gazzano-Santoro et al., J. Immunol. Methods 202: 163 (1996), may be performed. Polypeptide variants with altered Fc region amino acid sequences (polypeptides with a variant Fc region) and increased or decreased C1q binding are described, e.g., in U.S. Pat. No. 6,194,551 Bi and WO 1999/51642.

“Percent (%) sequence identity” with respect to a reference polynucleotide or polypeptide sequence, respectively, is defined as the percentage of nucleic acid or amino acid residues, respectively, in a candidate sequence that are identical with the nucleic acid or amino acid residues, respectively, in the reference polynucleotide or polypeptide sequence, respectively, after aligning the sequences and introducing gaps, if necessary, to achieve the maximum percent sequence identity. Conservative substitutions are not considered as part of the sequence identity. Preferred are un-gapped alignments. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full-length of the sequences being compared.

The terms “polynucleotide” or “nucleic acid”, as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs.

“Sequence homology” indicates the percentage of amino acids that either is identical or that represent conservative amino acid substitutions.

The term “maturated antibodies” or “maturated antigen-binding fragments” such as maturated Fab variants includes derivatives of an antibody or antibody fragment exhibiting stronger binding—i. e. binding with increased affinity—to a given antigen such as the extracellular domain of a target protein. Maturation is the process of identifying a small number of mutations e.g. within the six CDRs of an antibody or antibody fragment leading to this affinity increase. The maturation process is the combination of molecular biology methods for introduction of mutations into the antibody and screening for identifying the improved binders.

The term “pharmaceutical formulation”/“pharmaceutical composition” refers to a preparation which is in such form as to permit the biological activity of an active ingredient contained therein to be effective, and which contains no additional components which are unacceptably toxic to a subject to which the formulation would be administered.

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell”, “host cell line”, and “host cell culture” are used interchangeably and refer to cells into which exogenous nucleic acid has been introduced, including the progeny of such cells. Host cells include “transformants”, “transformed cells”, “transfectants”, “transfected cells”, and “transduced cells”, which include the primary transformed/transfected/transduced cell and progeny derived therefrom without regard to the number of passages. Progeny may not be completely identical in nucleic acid content to a parent cell but may contain mutations. Mutant progeny that have the same function or biological activity as screened or selected for in the originally transformed cell are included herein.

The term “reversal agent” as used herein, refers to a protein, polypeptide, or a complex thereof, such as an antigen binding antibody (e.g. a full-length antibody or a monovalent antibody) or a fragment thereof, such as a Fab fragment, or an inactive FXI/FXIa-derived polypeptide or protein fragment that specifically binds to an anti-FXIa antibody, preferentially anti FXIa-antibody 076D-M007-H04-CDRL3-N110D as described in WO2013/167669. In specific aspects provided herein, the reversal agent is capable of neutralizing (e.g. partially neutralizing by at least 10%, at least 20%, at least 30%, at least 40%, at least 50%, at least 60%, at least 70%, at least 80%) the therapeutic activity of this anti-FXIa antibody.

Reversal Agents of this Invention

The present invention is related to novel reversal agents, which specifically bind to the anti-FXIa antibody 076D-M007-H04-CDRL3-N110D as described in WO2013/167669 and neutralize the therapeutic activity of this anti-FXIa antibody. The reversal agents of the invention, which may be human, humanized or chimeric antibodies, such as full-length antibodies or monovalent antibodies, or antigen-binding fragments thereof, such as Fab fragments, can be used in many contexts, which are more fully described herein.

Throughout this document, reference is made to the following antibodies or antigen-binding fragments thereof of the invention, which specifically bind to the anti-FXIa antibody

TABLE 1
Protein sequences of antibodies and antigen-binding fragments thereof according to this invention

| | | | | | | | | | SEQ | SEQ | SEQ |
|---|---|---|---|---|---|---|---|---|---|---|---|
| | | | | | | | | | ID NO: | ID NO: | ID NO: |
| | SEQ | SEQ | SEQ | SEQ | SEQ | SEQ | SEQ | SEQ | IgG/fab | IgG/Fab | IgG/fab |
| | ID NO: | ID NO: | ID NO: | ID NO: | ID NO: | ID NO: | ID NO: | ID NO: | Heavy | Light | Heavy |
| | VH Protein | H-CDR1 | H-CDR2 | H-CDR3 | VL Protein | L-CDR1 | L-CDR2 | L-CDR3 | Chain | Chain | Chain 2 |
| TPP-8236 | 1 | 2 | 3 | 4 | 5 | 6 | 7 | 8 | 11 | 12 | |
| TPP-8237 | 15 | 16 | 17 | 18 | 19 | 20 | 21 | 22 | 25 | 26 | |
| TPP-8238 | 29 | 30 | 31 | 32 | 33 | 34 | 35 | 36 | 39 | 40 | |
| TPP-8239 | 43 | 44 | 45 | 46 | 47 | 48 | 49 | 50 | 53 | 54 | |
| TPP-8240 | 57 | 58 | 59 | 60 | 61 | 62 | 63 | 64 | 67 | 68 | |
| TPP-8241 | 71 | 72 | 73 | 74 | 75 | 76 | 77 | 78 | 81 | 82 | |
| TPP-8243 | 85 | 86 | 87 | 88 | 89 | 90 | 91 | 92 | 95 | 96 | |
| TPP-8246 | 99 | 100 | 101 | 102 | 103 | 104 | 105 | 106 | 109 | 110 | |
| TPP-9238 | 113 | 114 | 115 | 116 | 117 | 118 | 119 | 120 | 123 | 124 | |
| TPP-9251 | 127 | 128 | 129 | 130 | 131 | 132 | 133 | 134 | 137 | 138 | |
| TPP-9252 | 141 | 142 | 143 | 144 | 145 | 146 | 147 | 148 | 151 | 152 | |
| TPP-9258 | 155 | 156 | 157 | 158 | 159 | 160 | 161 | 162 | 165 | 166 | |
| TPP-10089 | 169 | 170 | 171 | 172 | 173 | 174 | 175 | 176 | 179 | 180 | |
| TPP-20816 | 183 | 184 | 185 | 186 | 187 | 188 | 189 | 190 | 191 | 192 | 193 |

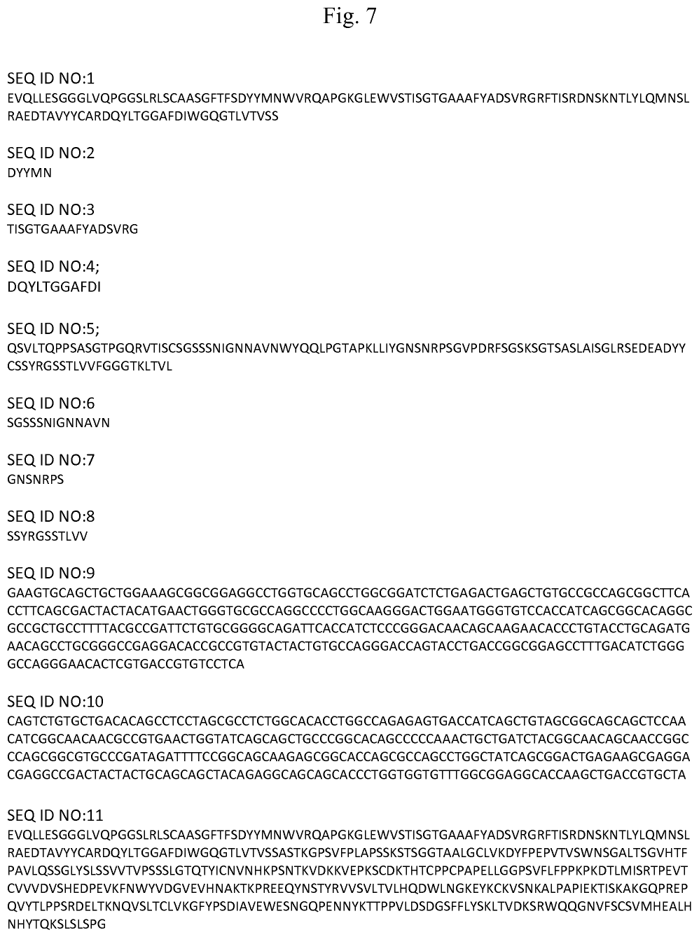

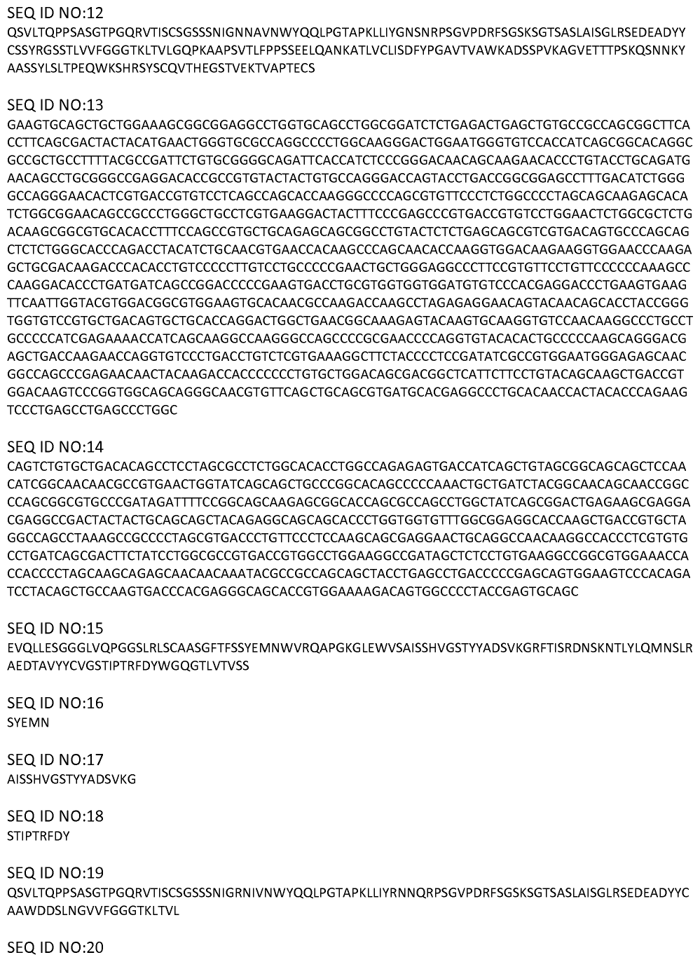

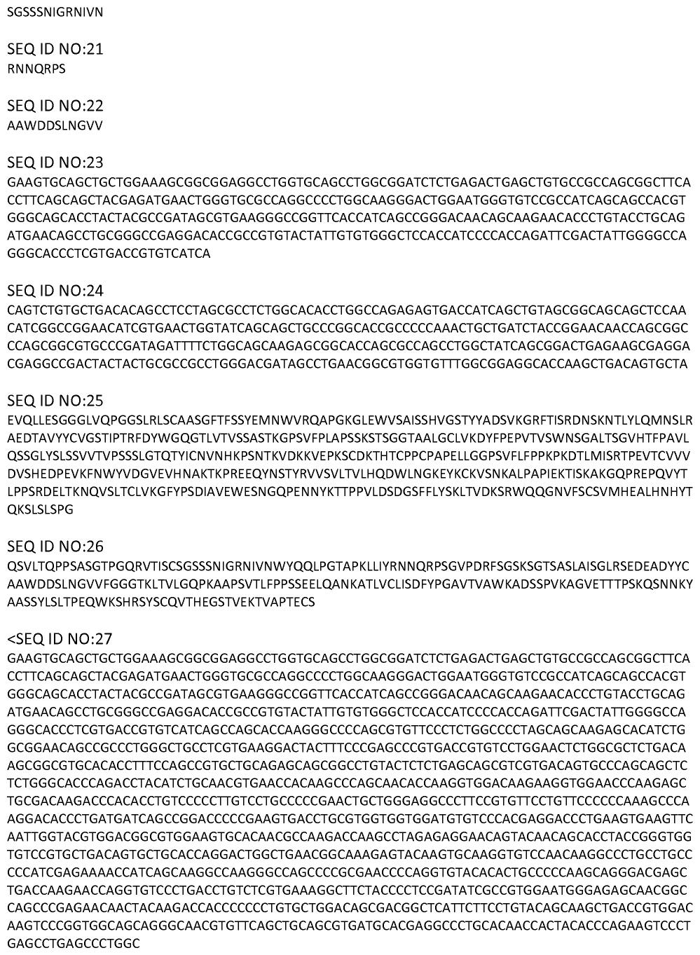

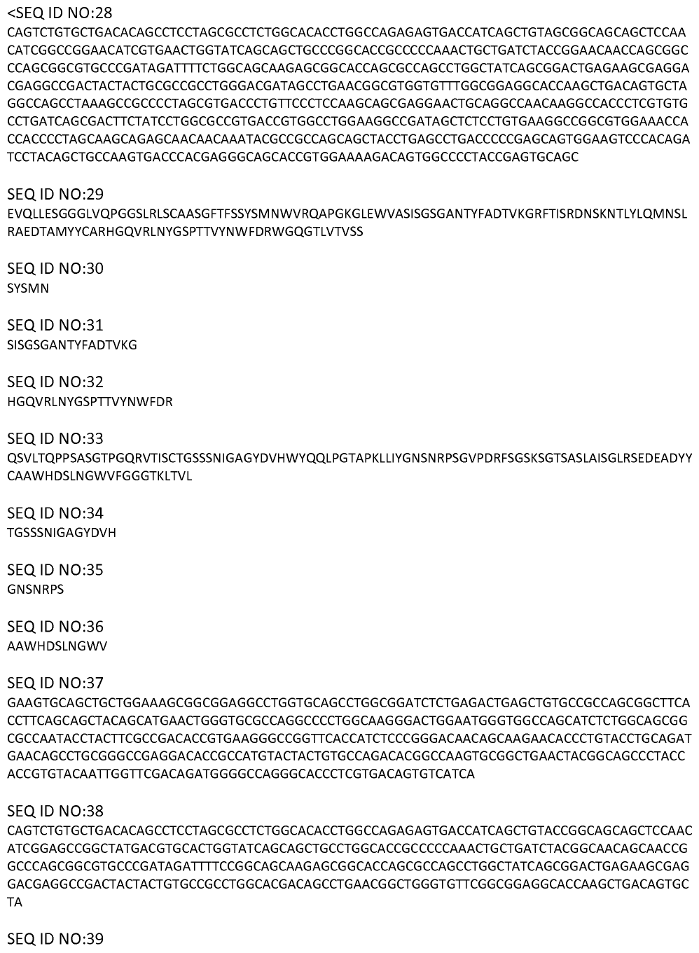

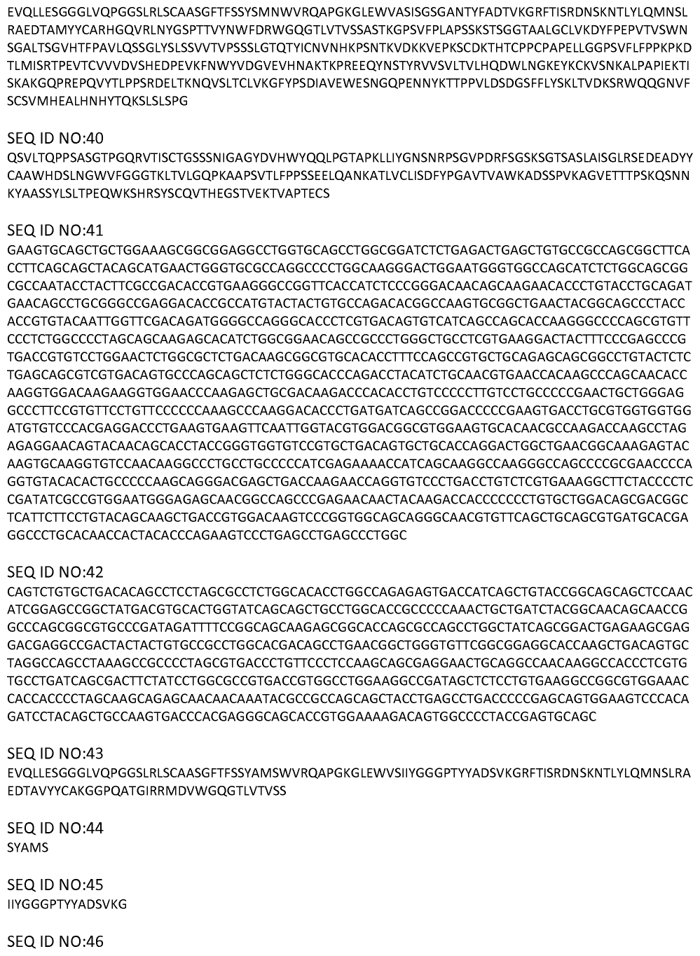

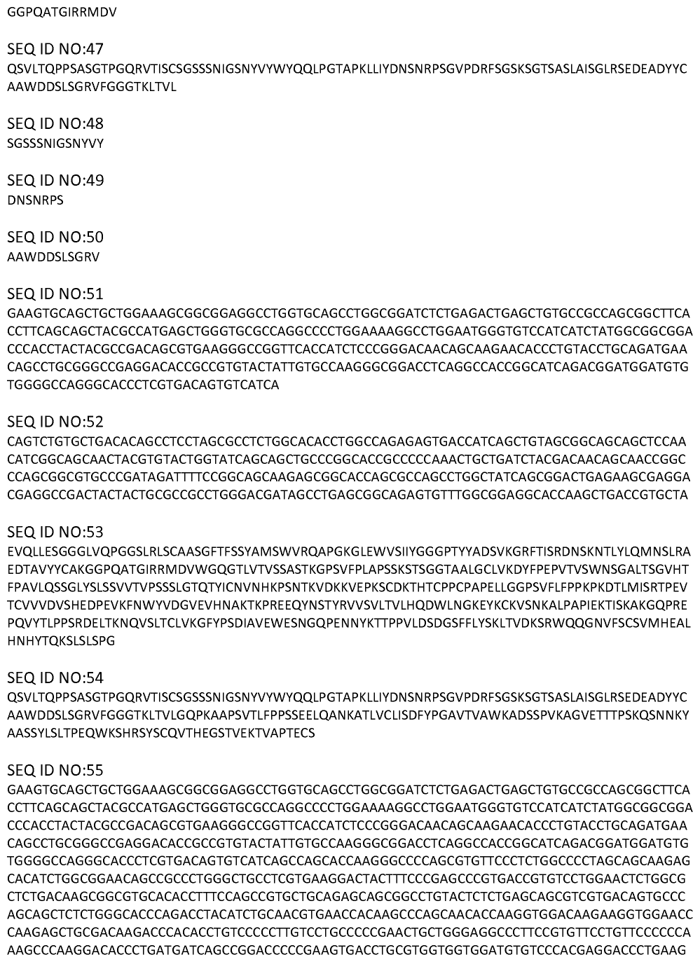

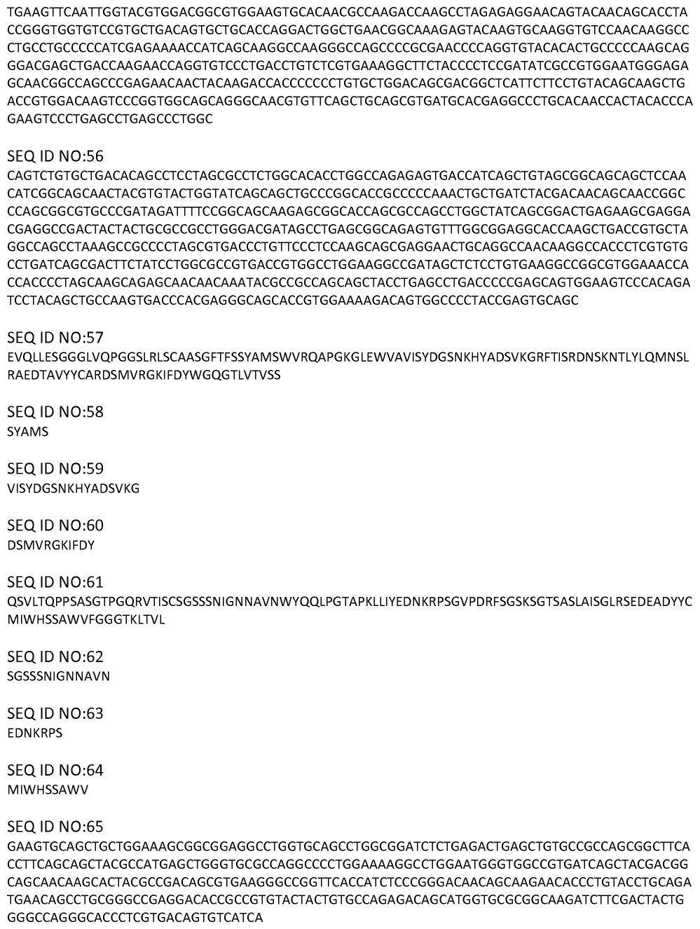

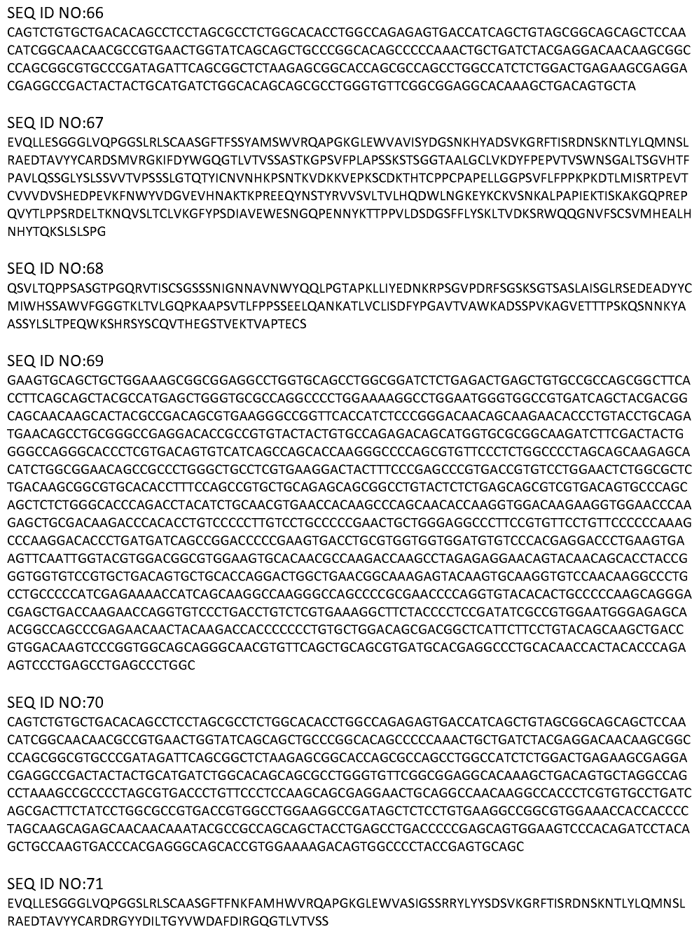

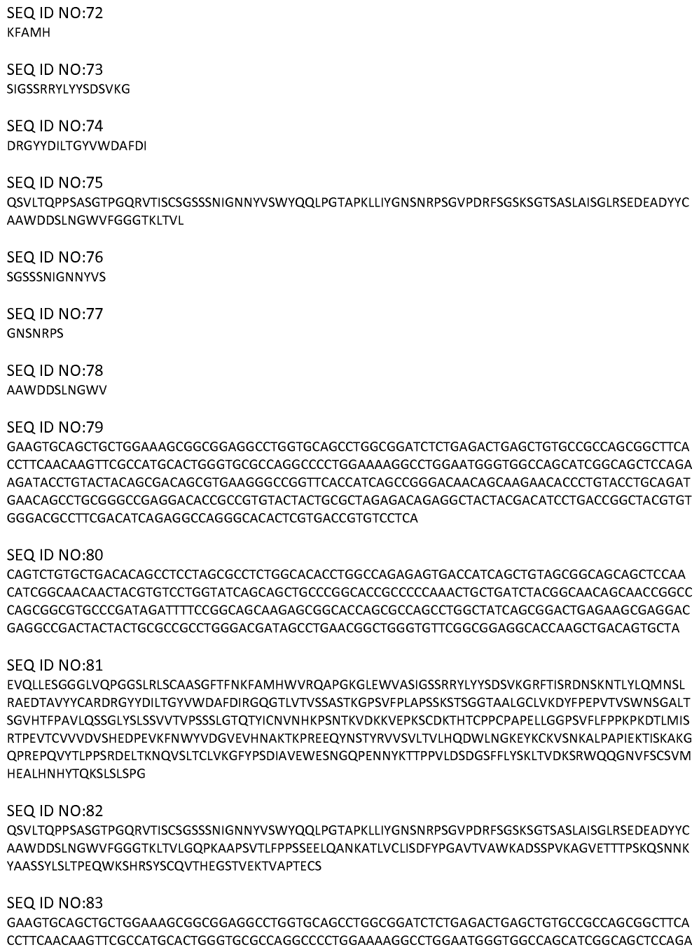

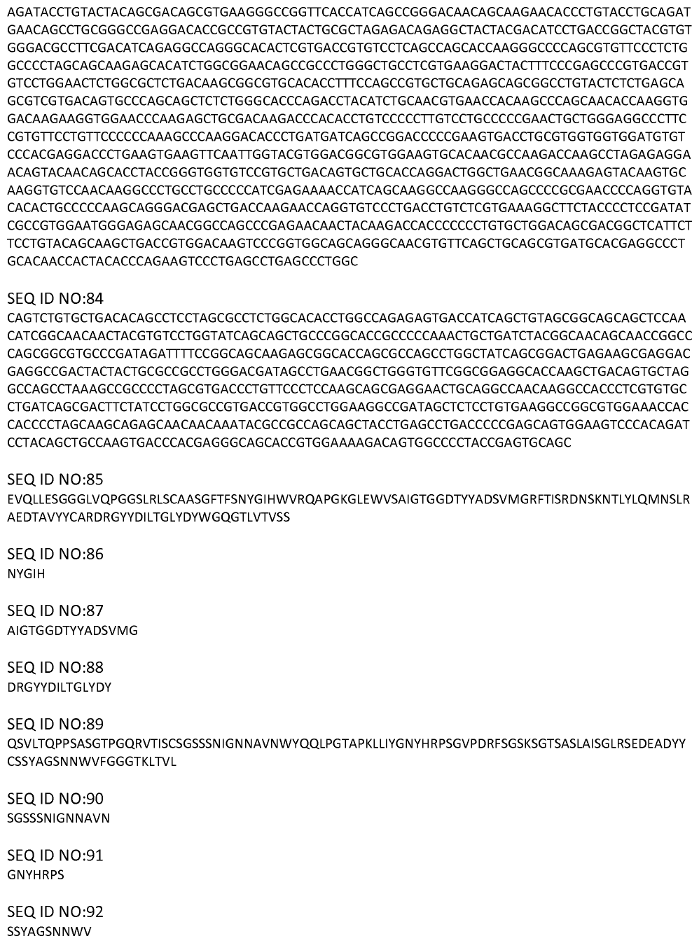

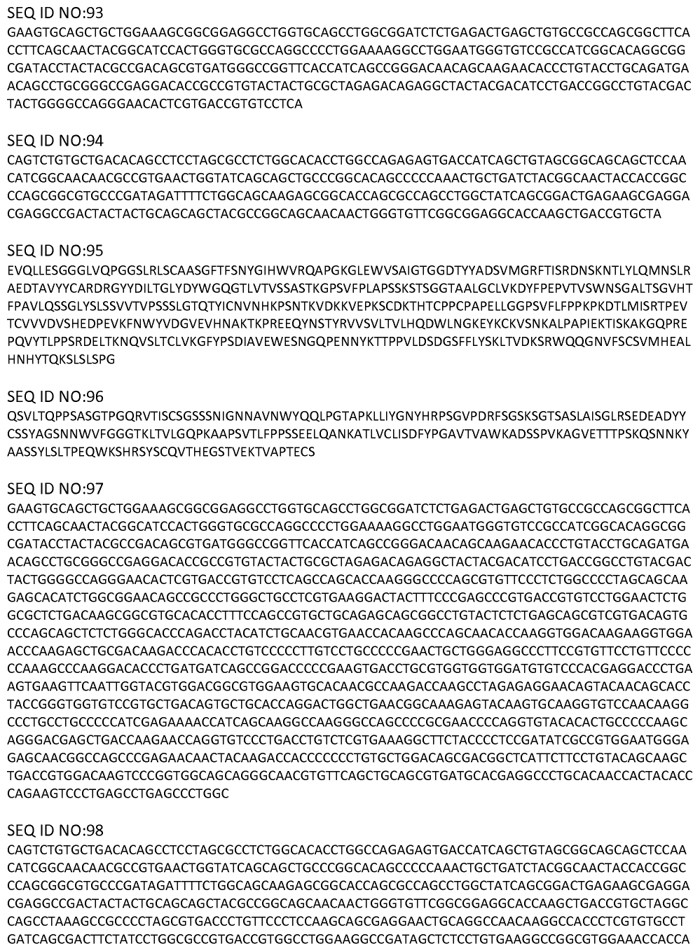

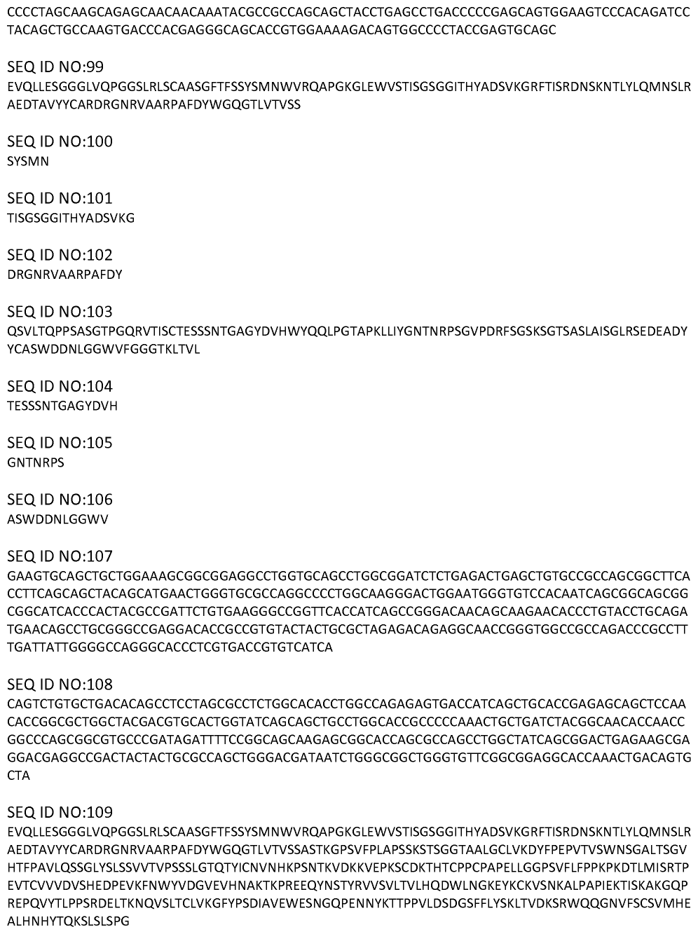

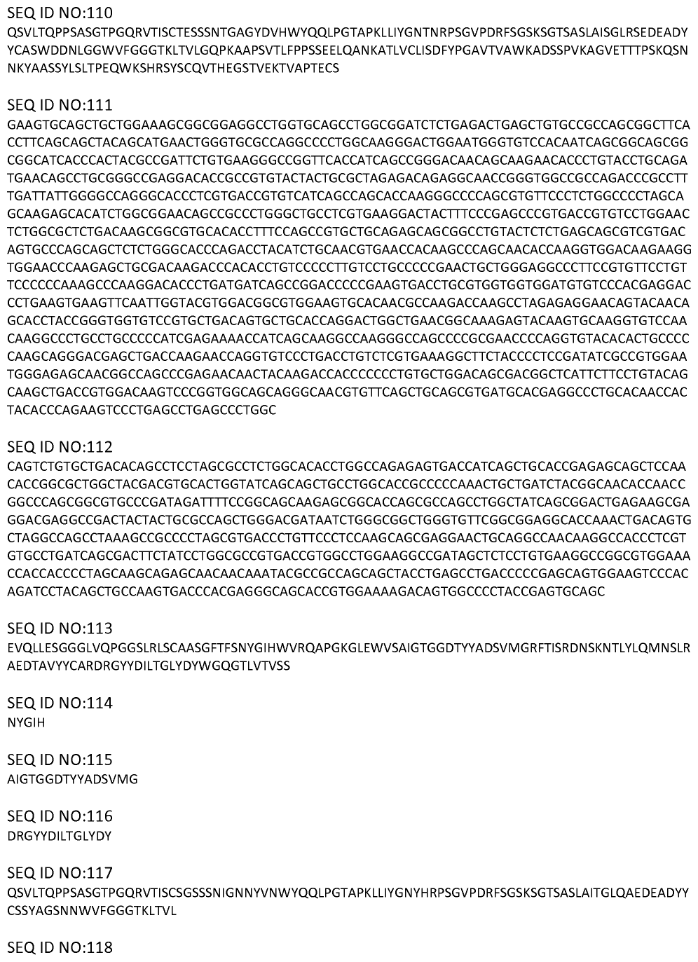

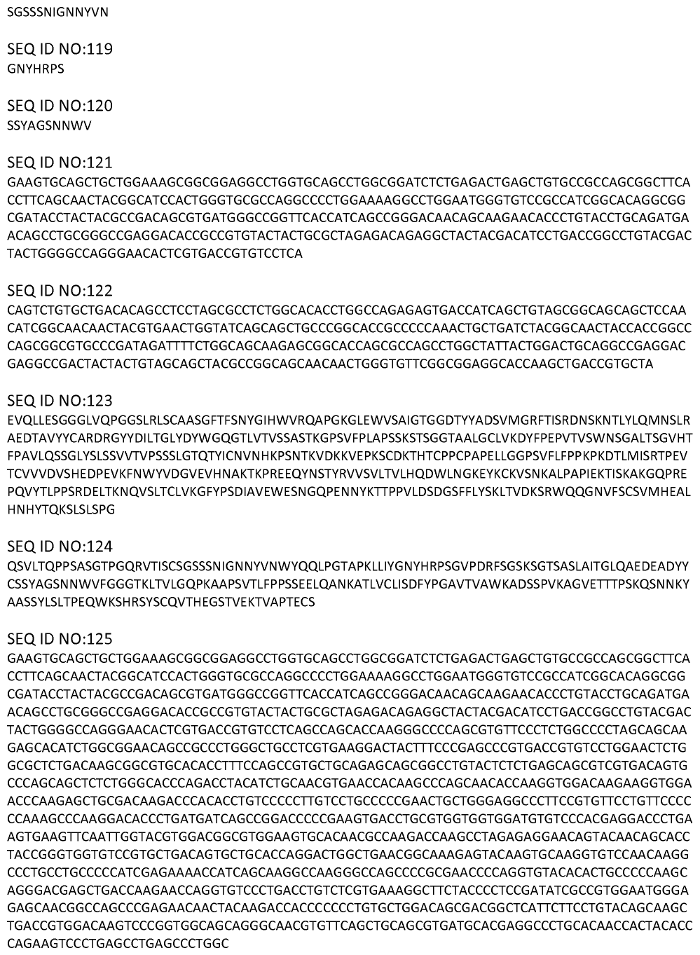

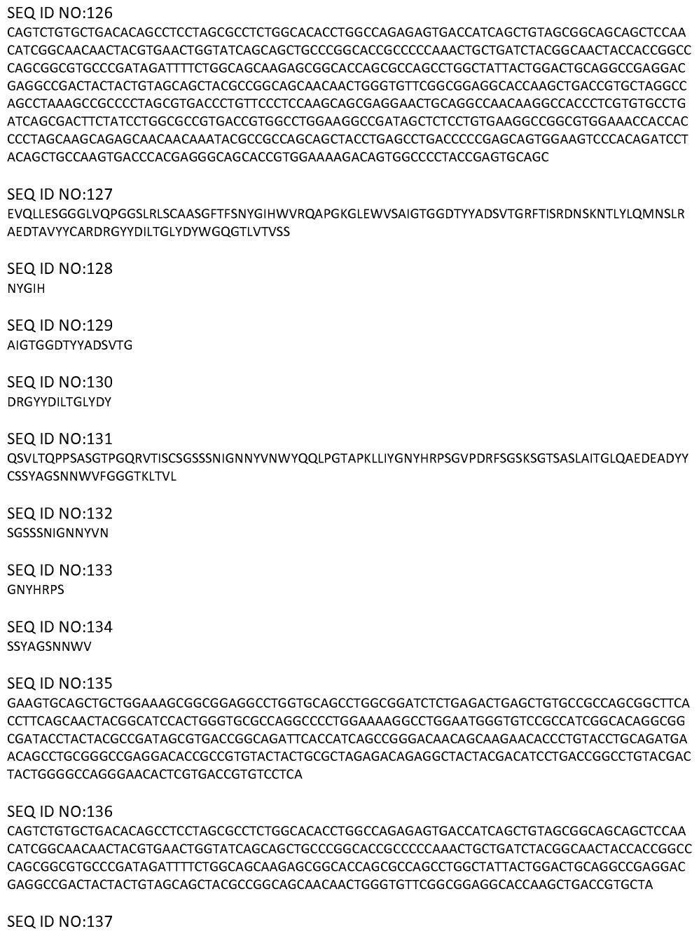

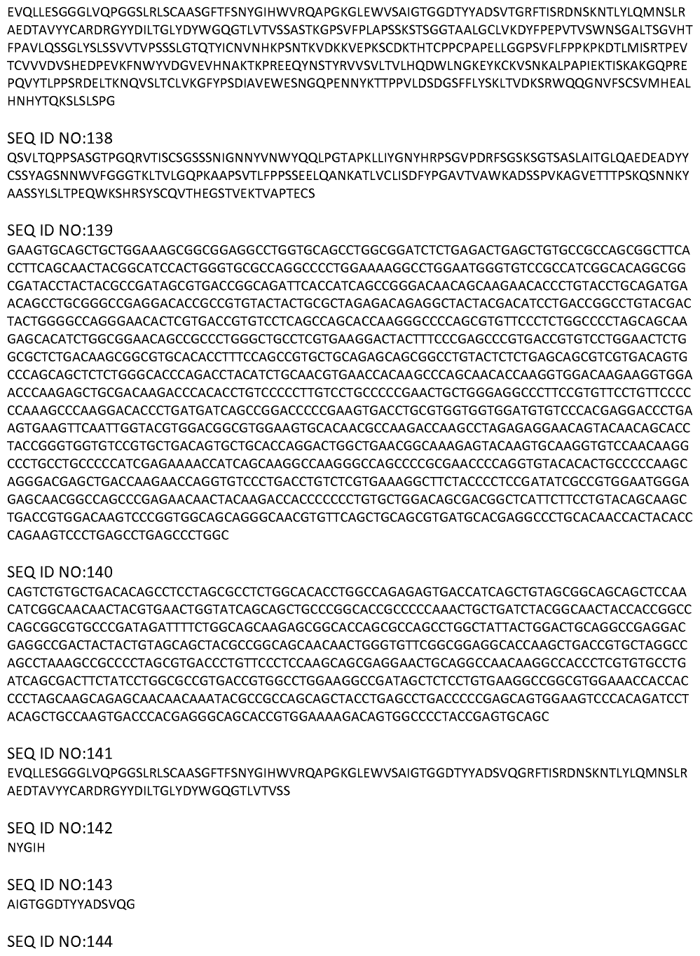

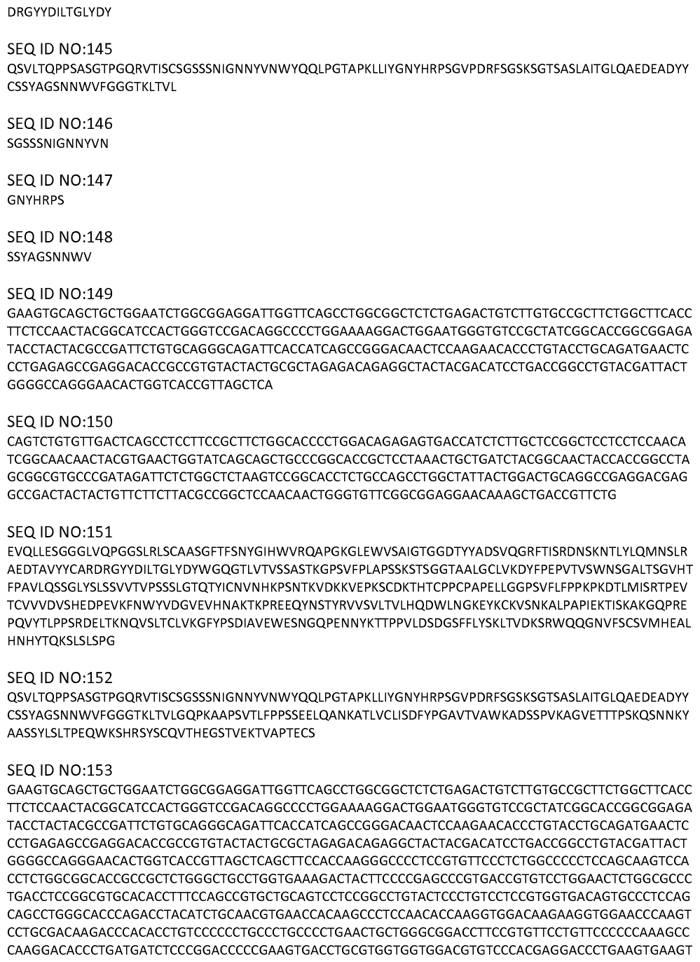

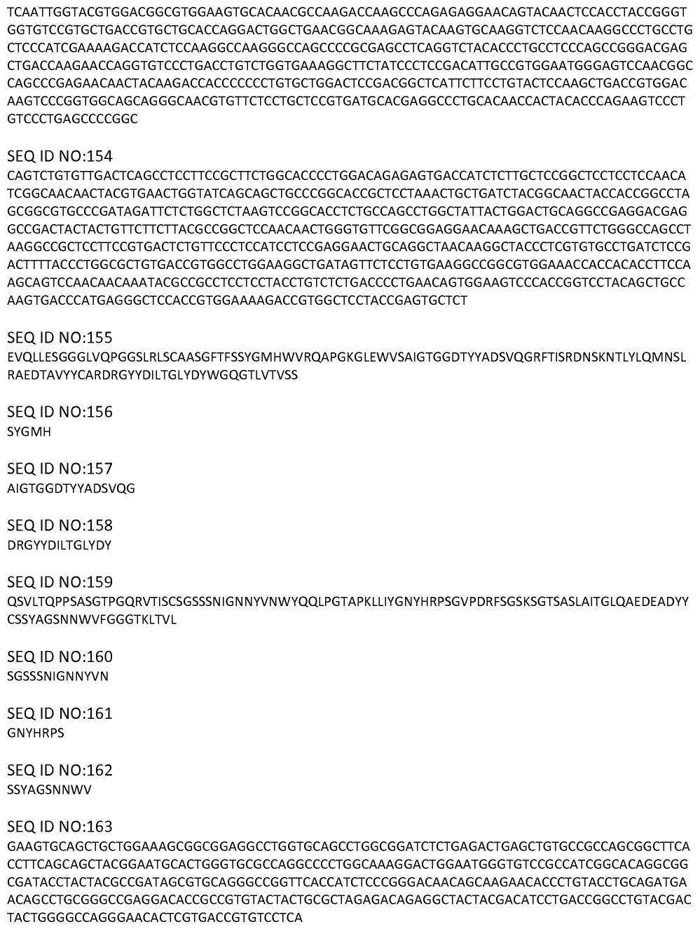

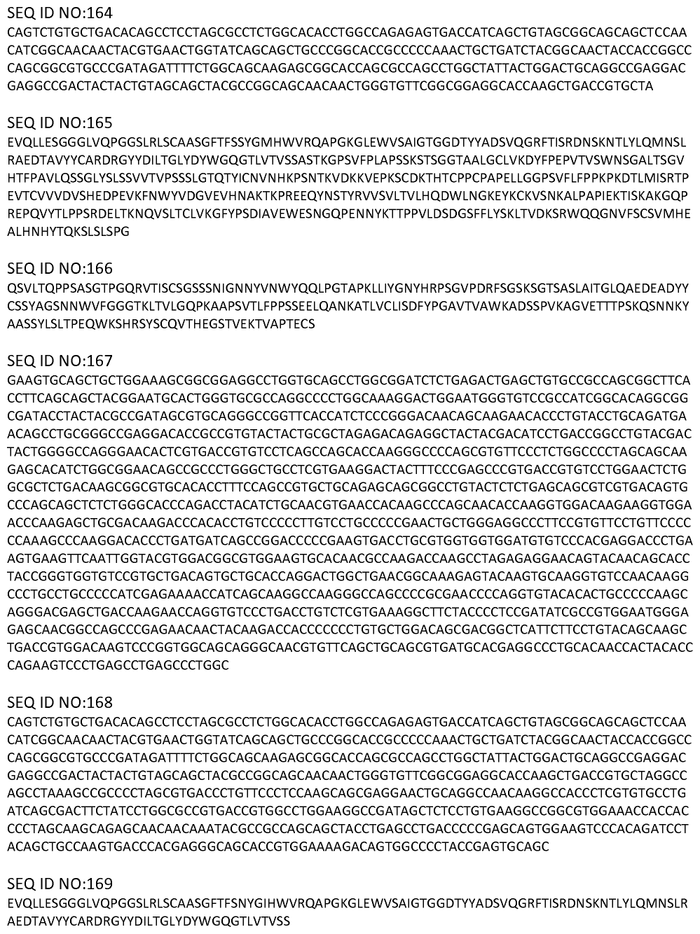

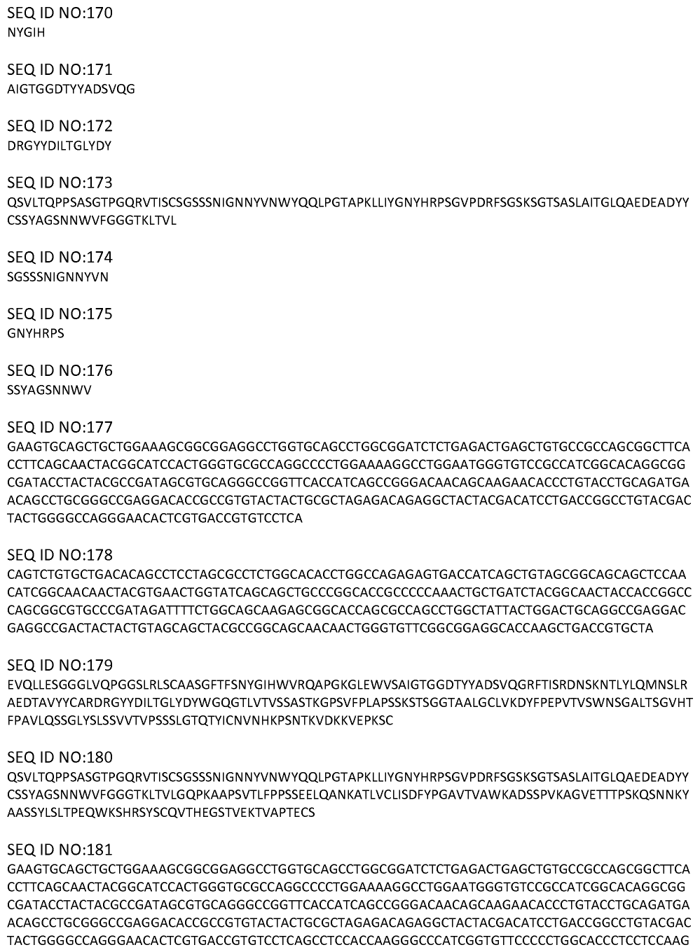

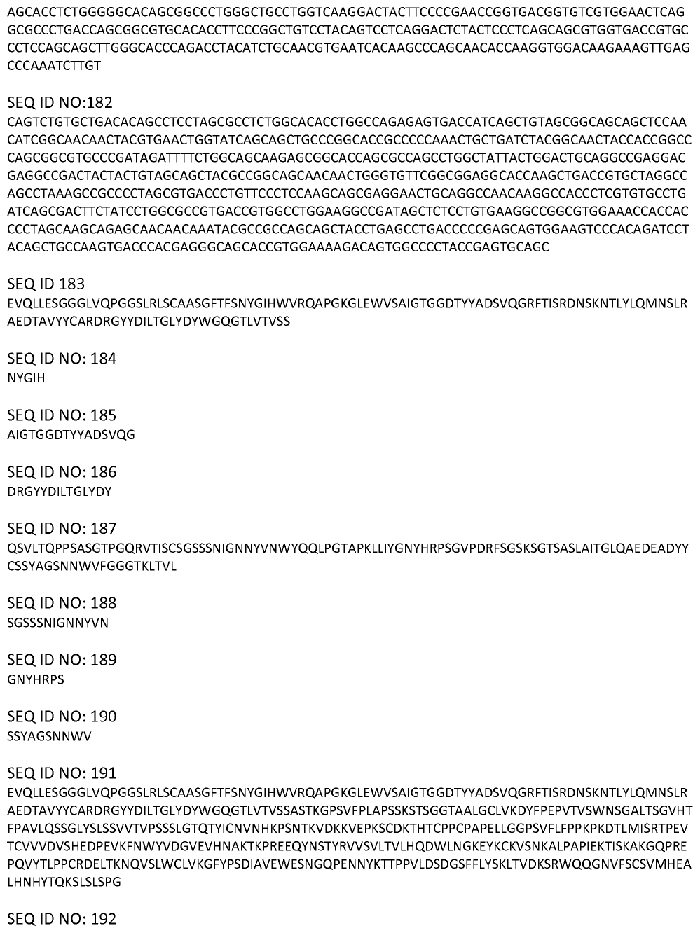

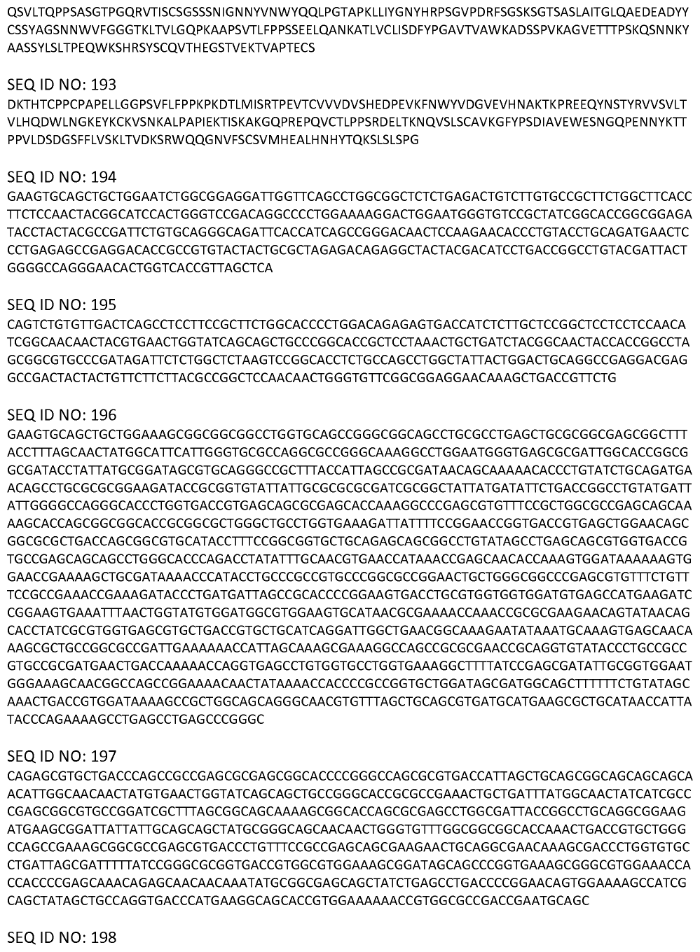

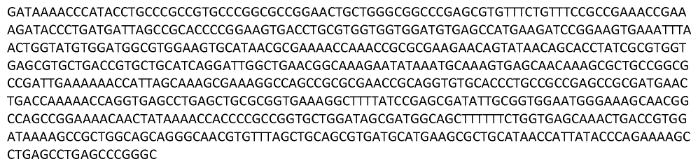

The sequences of antibodies of this invention or antigen-binding fragments thereof as depicted in Table 1 are further provided and explained in FIG. 7.

TPP-8236 represents an antibody comprising a heavy chain region corresponding to SEQ ID NO: 11 and a light chain region corresponding to SEQ ID NO: 12.

TPP-8237 represents an antibody comprising a heavy chain region corresponding to SEQ ID NO: 25 and a light chain region corresponding to SEQ ID NO: 26.

TPP-8238 represents an antibody comprising a heavy chain region corresponding to SEQ ID NO: 39 and a light chain region corresponding to SEQ ID NO: 40.

TPP-8239 represents an antibody comprising a heavy chain region corresponding to SEQ ID NO: 53 and a light chain region corresponding to SEQ ID NO: 54.

TPP-8240 represents an antibody comprising a heavy chain region corresponding to SEQ ID NO: 67 and a light chain region corresponding to SEQ ID NO: 68.

TPP-8241 represents an antibody comprising a heavy chain region corresponding to SEQ ID NO: 81 and a light chain region corresponding to SEQ ID NO: 82.

TPP-8243 represents an antibody comprising a heavy chain region corresponding to SEQ ID NO: 95 and a light chain region corresponding to SEQ ID NO: 96.

TPP-8246 represents an antibody comprising a heavy chain region corresponding to SEQ ID NO: 109 and a light chain region corresponding to SEQ ID NO: 110.

TPP-9238 represents an antibody comprising a heavy chain region corresponding to SEQ ID NO: 123 and a light chain region corresponding to SEQ ID NO: 124.

TPP-9251 represents an antibody comprising a heavy chain region corresponding to SEQ ID NO: 137 and a light chain region corresponding to SEQ ID NO: 138.

TPP-9252 represents an antibody comprising a heavy chain region corresponding to SEQ ID NO: 151 and a light chain region corresponding to SEQ ID NO: 152.

TPP-9258 represents an antibody comprising a heavy chain region corresponding to SEQ ID NO: 165 and a light chain region corresponding to SEQ ID NO: 166.

TPP-10089 represents a Fab fragment of full-length IgG TPP-9252 comprising a heavy chain region corresponding to SEQ ID NO: 179 and a light chain region corresponding to SEQ ID NO: 180.

TPP-20816 represents a monovalent antibody derived (by the so-called ‘knobs-into-holes’ technology) from full-length IgG TPP-9252 comprising heavy chain regions corresponding to SEQ ID NO: 191 and 193 and a light chain region corresponding to SEQ ID NO: 192.

In a further embodiment the antibodies or antigen-binding fragments comprise heavy or light chain CDR sequences which are at least 50%, 55%, 60% 70%, 80%, 90, or 95% identical to at least one, preferably corresponding, CDR sequence of the antibodies “TPP-8236”, “TPP-8237”, “TPP-8238”, “TPP-8239”, “TPP-8240”, “TPP-8241”, “TPP-8343”, “TPP-8246”, “TPP-9238”, “TPP-9251”, “TPP-9252”, “TPP-9258” or fab fragment “TPP-10089” or at least 50%, 60%, 70%, 80%, 90%, 92% or 95% identical to the VH or VL sequence of TPP-8236”, “TPP-8237”, “TPP-8238”, “TPP-8239”, “TPP-8240”, “TPP-8241”, “TPP-8343”, “TPP-8246”, “TPP-9238”, “TPP-9251”, “TPP-9252”, “TPP-9258”, Fab fragment “TPP-10089”, or monovalent antibody “TPP-20816”, respectively.

In a further embodiment the antibody of the invention or antigen-binding fragment thereof comprises at least one CDR sequence or at least one variable heavy chain or variable light chain sequence as depicted in Table 1.

In an embodiment the antibody of the invention or antigen-binding fragment thereof comprises a heavy chain antigen-binding region that comprises SEQ ID NO:2 (H-CDR1), SEQ ID NO:3 (H-CDR2) and SEQ ID NO:4 (H-CDR3) and comprises a light chain antigen-binding region that comprises SEQ ID NO:6 (L-CDR1), SEQ ID NO:7 (L-CDR2) and SEQ ID NO:8 (L-CDR3).

In an embodiment the antibody of the invention or antigen-binding fragment thereof comprises a heavy chain antigen-binding region that comprises SEQ ID NO:16 (H-CDR1), SEQ ID NO:17 (H-CDR2) and SEQ ID NO:18 (H-CDR3) and comprises a light chain antigen-binding region that comprises SEQ ID NO:20 (L-CDR1), SEQ ID NO:21 (L-CDR2) and SEQ ID NO:22 (L-CDR3).

In an embodiment the antibody of the invention or antigen-binding fragment thereof comprises a heavy chain antigen-binding region that comprises SEQ ID NO:30 (H-CDR1), SEQ ID NO:31 (H-CDR2) and SEQ ID NO:32 (H-CDR3) and comprises a light chain antigen-binding region that comprises SEQ ID NO:34 (L-CDR1), SEQ ID NO:35 (L-CDR2) and SEQ ID NO:36 (L-CDR3).

In an embodiment the antibody of the invention or antigen-binding fragment thereof comprises a heavy chain antigen-binding region that comprises SEQ ID NO:44 (H-CDR1), SEQ ID NO:45 (H-CDR2) and SEQ ID NO:46 (H-CDR3) and comprises a light chain antigen-binding region that comprises SEQ ID NO:48 (L-CDR1), SEQ ID NO:49 (L-CDR2) and SEQ ID NO:50 (L-CDR3).

In an embodiment the antibody of the invention or antigen-binding fragment thereof comprises a heavy chain antigen-binding region that comprises SEQ ID NO:58 (H-CDR1), SEQ ID NO:59 (H-CDR2) and SEQ ID NO:60 (H-CDR3) and comprises a light chain antigen-binding region that comprises SEQ ID NO:62 (L-CDR1), SEQ ID NO:63 (L-CDR2) and SEQ ID NO:64 (L-CDR3).

In an embodiment the antibody of the invention or antigen-binding fragment thereof comprises a heavy chain antigen-binding region that comprises SEQ ID NO:72 (H-CDR1), SEQ ID NO:73 (H-CDR2) and SEQ ID NO:74 (H-CDR3) and comprises a light chain antigen-binding region that comprises SEQ ID NO:76 (L-CDR1), SEQ ID NO:77 (L-CDR2) and SEQ ID NO:78 (L-CDR3).

In an embodiment the antibody of the invention or antigen-binding fragment thereof comprises a heavy chain antigen-binding region that comprises SEQ ID NO:86 (H-CDR1), SEQ ID NO:87 (H-CDR2) and SEQ ID NO:88 (H-CDR3) and comprises a light chain antigen-binding region that comprises SEQ ID NO:90 (L-CDR1), SEQ ID NO:91 (L-CDR2) and SEQ ID NO:92 (L-CDR3).

In an embodiment the antibody of the invention or antigen-binding fragment thereof comprises a heavy chain antigen-binding region that comprises SEQ ID NO:100 (H-CDR1), SEQ ID NO:101 (H-CDR2) and SEQ ID NO:102 (H-CDR3) and comprises a light chain antigen-binding region that comprises SEQ ID NO:104 (L-CDR1), SEQ ID NO:105 (L-CDR2) and SEQ ID NO:106 (L-CDR3).

In an embodiment the antibody of the invention or antigen-binding fragment thereof comprises a heavy chain antigen-binding region that comprises SEQ ID NO:114 (H-CDR1), SEQ ID NO:115 (H-CDR2) and SEQ ID NO:116 (H-CDR3) and comprises a light chain antigen-binding region that comprises SEQ ID NO:118 (L-CDR1), SEQ ID NO:119 (L-CDR2) and SEQ ID NO:120 (L-CDR3).

In an embodiment the antibody of the invention or antigen-binding fragment thereof comprises a heavy chain antigen-binding region that comprises SEQ ID NO:128 (H-CDR1), SEQ ID NO:129 (H-CDR2) and SEQ ID NO:130 (H-CDR3) and comprises a light chain antigen-binding region that comprises SEQ ID NO:132 (L-CDR1), SEQ ID NO:133 (L-CDR2) and SEQ ID NO:134 (L-CDR3).

In an embodiment the antibody of the invention or antigen-binding fragment thereof comprises a heavy chain antigen-binding region that comprises SEQ ID NO:142 (H-CDR1), SEQ ID NO:143 (H-CDR2) and SEQ ID NO:144 (H-CDR3) and comprises a light chain antigen-binding region that comprises SEQ ID NO:146 (L-CDR1), SEQ ID NO:147 (L-CDR2) and SEQ ID NO:148 (L-CDR3).

In an embodiment the antibody of the invention or antigen-binding fragment thereof comprises a heavy chain antigen-binding region that comprises SEQ ID NO:156 (H-CDR1), SEQ ID NO:157 (H-CDR2) and SEQ ID NO:158 (H-CDR3) and comprises a light chain antigen-binding region that comprises SEQ ID NO:160 (L-CDR1), SEQ ID NO:161 (L-CDR2) and SEQ ID NO:162 (L-CDR3).

In an embodiment the antibody of the invention or antigen-binding fragment thereof comprises a heavy chain antigen-binding region that comprises SEQ ID NO:170 (H-CDR1), SEQ ID NO:171 (H-CDR2) and SEQ ID NO:172 (H-CDR3) and comprises a light chain antigen-binding region that comprises SEQ ID NO:174 (L-CDR1), SEQ ID NO:175 (L-CDR2) and SEQ ID NO:176 (L-CDR3).

In an embodiment the antibody of the invention or antigen-binding fragment thereof comprises a heavy chain antigen-binding region that comprises SEQ ID NO:184 (H-CDR1), SEQ ID NO:186 (H-CDR2) and SEQ ID NO:186 (H-CDR3) and comprises a light chain antigen-binding region that comprises SEQ ID NO:188 (L-CDR1), SEQ ID NO:189 (L-CDR2) and SEQ ID NO:190 (L-CDR3).

Antibodies differ in sequence, not only within their complementarity determining regions (CDRs), but also in the framework (FR). These sequence differences are encoded in the different V-genes. The human antibody germline repertoire has been completely sequenced.

There are about 50 functional VH germline genes which can be grouped into six subfamilies according to sequence homology VH1, VH2, VH3, VH4, VH5 and VH6 (Tomlinson et al., 1992, J. Mol. Biol. 227, 776-798; Matsuda & Honjo, 1996, Advan. Immunol. 62, 1-29). The length of a light chain protein ranges from 211 to 217 amino acids. The constant region determines what class—either kappa or lambda—the light chain is. The lambda class has 4 subtypes (Owen, Judith A.; Punt, Jenni; Stranford, Sharon (2013). Kuby Immunology. New York, N.Y.: W. H. Freeman and Company). Disclosed herein are heavy chains of antibodies of this invention that belong to the human VH3 subfamily and the light chains of antibodies of this invention that belong to the human Vkappa1lambda subfamily, respectively. It is known that framework sequences of antibodies belonging to the same subfamily are closely related, e.g. antibodies comprising a human VH3 subfamily member all share comparable stability (Honegger et al., 2009, Protein Eng Des Sel. 22(3):121-134). It is well known in the art that CDRs from antibodies can be grafted on different frameworks while maintaining special features of the corresponding origin antibody. CDRs have been successfully grafted on frameworks belonging to a different species as well as on frameworks of the same species belonging to a different subfamily. In a further embodiment the antibody or antigen-binding fragment of the invention comprises at least one CDR sequence of antibody of the invention as depicted in Table 1 and a human variable chain framework sequence.

In a preferred embodiment the antibody or antigen-binding fragment of the invention comprises a variable light chain or light chain antigen-binding region comprising the L-CDR1, L-CDR2 and L-CDR3 sequence of the variable light chain and a variable heavy chain or heavy chain antigen-binding region comprising the H-CDR1, H-CDR2 and H-CDR3 sequence of the variable heavy chain antibody of the invention as depicted in Table 1 and a human variable light and human variable heavy chain framework sequence.

In a preferred embodiment the antibody of the invention or antigen-binding fragment thereof comprises a variable heavy chain sequence as presented by SEQ ID NO:1 (VH) and a variable light chain sequences as presented by SEQ ID NO:5 (VL).

In a preferred embodiment the antibody of the invention or antigen-binding fragment thereof comprises a variable heavy chain sequence as presented by SEQ ID NO:15 (VH) and a variable light chain sequences as presented by SEQ ID NO:19 (VL).

In a preferred embodiment the antibody of the invention or antigen-binding fragment thereof comprises a variable heavy chain sequence as presented by SEQ ID NO:29 (VH) and a variable light chain sequences as presented by SEQ ID NO:33 (VL).

In a preferred embodiment the antibody of the invention or antigen-binding fragment thereof comprises a variable heavy chain sequence as presented by SEQ ID NO:43 (VH) and a variable light chain sequences as presented by SEQ ID NO:47 (VL).

In a preferred embodiment the antibody of the invention or antigen-binding fragment thereof comprises a variable heavy chain sequence as presented by SEQ ID NO:57 (VH) and a variable light chain sequences as presented by SEQ ID NO:61 (VL).

In a preferred embodiment the antibody of the invention or antigen-binding fragment thereof comprises a variable heavy chain sequence as presented by SEQ ID NO:71 (VH) and a variable light chain sequences as presented by SEQ ID NO:75 (VL).

In a preferred embodiment the antibody of the invention or antigen-binding fragment thereof comprises a variable heavy chain sequence as presented by SEQ ID NO:85 (VH) and a variable light chain sequences as presented by SEQ ID NO:89 (VL).

In a preferred embodiment the antibody of the invention or antigen-binding fragment thereof comprises a variable heavy chain sequence as presented by SEQ ID NO:99 (VH) and a variable light chain sequences as presented by SEQ ID NO:103 (VL).

In a preferred embodiment the antibody of the invention or antigen-binding fragment thereof comprises a variable heavy chain sequence as presented by SEQ ID NO:113 (VH) and a variable light chain sequences as presented by SEQ ID NO:117 (VL).

In a preferred embodiment the antibody of the invention or antigen-binding fragment thereof comprises a variable heavy chain sequence as presented by SEQ ID NO:127 (VH) and a variable light chain sequences as presented by SEQ ID NO:131 (VL).

In a preferred embodiment the antibody of the invention or antigen-binding fragment thereof comprises a variable heavy chain sequence as presented by SEQ ID NO:141 (VH) and a variable light chain sequences as presented by SEQ ID NO:145 (VL).

In a preferred embodiment the antibody of the invention or antigen-binding fragment thereof comprises a variable heavy chain sequence as presented by SEQ ID NO:155 (VH) and a variable light chain sequences as presented by SEQ ID NO:159 (VL).

In a preferred embodiment the antibody of the invention or antigen-binding fragment thereof comprises a variable heavy chain sequence as presented by SEQ ID NO:169 (VH) and a variable light chain sequences as presented by SEQ ID NO:173 (VL).

In a preferred embodiment the antibody of the invention or antigen-binding fragment thereof comprises a variable heavy chain sequence as presented by SEQ ID NO:183 (VH) and a variable light chain sequences as presented by SEQ ID NO:187 (VL).

In some embodiments, the monoclonal antibody or antigen-binding fragment thereof that specifically binds to anti-FXIa antibody 076D-M007-H04-CDRL3-N110D is chimeric, humanized, or human.

In some embodiments, the monoclonal antibody or antigen-binding fragment thereof that specifically binds to anti-FXIa antibody 076D-M007-H04-CDRL3-N110D comprises a human IgG heavy chain constant region.

In some embodiments, the monoclonal antibody or antigen-binding fragment thereof that specifically binds to anti-FXIa antibody 076D-M007-H04-CDRL3-N110D comprises a human IgG1 heavy chain constant region.

In some embodiments, the monoclonal antibody or antigen-binding fragment thereof that specifically binds to anti-FXIa antibody 076D-M007-H04-CDRL3-N110D comprises a human IgG2 and IgG4 heavy chain constant region, respectively.

In some embodiments, the antigen-binding fragment of the monoclonal antibody that specifically binds to anti-FXIa antibody 076D-M007-H04-CDRL3-N110D is a Fab fragment.

In some embodiments, the monoclonal antibody that specifically binds to anti-FXIa antibody 076D-M007-H04-CDRL3-N110D is a monovalent antibody and comprises a human IgG1 heavy chain constant region. In some preferred embodiments, the IgG1 heavy chain sequences of the monovalent antibody differ from the IgG1 heavy chain sequences of the full-length monoclonal antibody at certain positions in order to allow a specific and exclusive complex formation of the heavy chain fused to the Fab sequence and the heavy chain without any Fab sequence. Preferably, these differences in the heavy chain sequences are achieved according to the so-called ‘knobs-into-holes’ technology, a well-validated heterodimerization technology for the third constant domain of an antibody as for example described in Ridgway et al. (1996) (‘Knobs-into-holes’ engineering of antibody CH3 domains for heavy chain heterodimerization. Protein Eng. 1996 July; 9(7):617-21)).

In some embodiments, the monoclonal antibody that specifically binds to anti-FXIa antibody 076D-M007-H04-CDRL3-N110D is a monovalent antibody and comprises a human IgG2 and IgG4 heavy chain constant region, respectively.

In certain preferred embodiments, the disclosure relates to a monoclonal antibody or antigen-binding fragment thereof that specifically binds to anti-FXIa antibody 076D-M007-H04-CDRL3-N110D and thereby inhibits the neutralizing activity of this anti-FXIa antibody, wherein the antibody or antigen binding fragment thereof comprises HCDR1-3 and LCDR1-3 comprising the amino acid sequences of:

a) SEQ ID NOs: 72, 73, 74, 76, 77, and 78, respectively;

b) SEQ ID NOs: 86, 87, 88, 90, 91, and 92, respectively;

c) SEQ ID NOs: 114, 115, 116, 118, 119, and 120, respectively;

d) SEQ ID NOs: 128, 129, 130, 132, 133, and 134, respectively;

e) SEQ ID NOs: 142, 143, 144, 146, 147, and 148, respectively;

f) SEQ ID NOs: 156, 157, 158, 160, 161, and 162, respectively;

g) SEQ ID NOs: 170, 171, 172, 174, 175, and 176, respectively; or

h) SEQ ID NOs: 184, 185, 186, 188, 189, and 190, respectively

In certain preferred embodiments, the disclosure relates to a monoclonal antibody or antigen-binding fragment thereof that specifically binds to anti-FXIa antibody 076D-M007-H04-CDRL3-N110D and thereby inhibits the neutralizing activity of this anti-FXIa antibody, wherein the antibody or antigen binding fragment thereof comprises a variable heavy chain (VH) sequence and a variable light chain (VL) sequence comprising the amino acid sequences of:

a) SEQ ID NOs: 71 and 75, respectively;

b) SEQ ID NOs: 85 and 89, respectively;

c) SEQ ID NOs: 113 and 117, respectively;

d) SEQ ID NOs: 127 and 131, respectively;

e) SEQ ID NOs: 141 and 145, respectively;

f) SEQ ID NOs: 155 and 159, respectively;

g) SEQ ID NOs: 169 and 173, respectively; or

h) SEQ ID NOs: 183 and 187, respectively

In certain preferred embodiments, the disclosure relates to a monoclonal antibody or antigen-binding fragment thereof that specifically binds to anti-FXIa antibody 076D-M007-H04-CDRL3-N110D and thereby inhibits the neutralizing activity of this anti-FXIa antibody, wherein the antibody or antigen binding fragment thereof comprises: a heavy chain sequence and a light chain sequence comprising the amino acid sequences of:

a) SEQ ID NOs: 81 and 82, respectively;

b) SEQ ID NOs: 95 and 96, respectively;

c) SEQ ID NOs: 123 and 124, respectively;

d) SEQ ID NOs: 137 and 138, respectively;

e) SEQ ID NOs: 151 and 152, respectively;

f) SEQ ID NOs: 165 and 166, respectively;

g) SEQ ID NOs: 179 and 180, respectively; or

h) SEQ ID NOs: 191, 192 and 193, respectively.

In certain especially preferred embodiments, the disclosure relates to a monoclonal antibody or antigen-binding fragment thereof that specifically binds to anti-FXIa antibody 076D-M007-H04-CDRL3-N110D and thereby inhibits the neutralizing activity of this anti-FXIa antibody, wherein the antibody or antigen binding fragment thereof comprises HCDR1-3 and LCDR1-3 comprising the amino acid sequences of SEQ ID NOs: 142, 143, 144, 146, 147, and 148, respectively; or of SEQ ID NOs: 170, 171, 172, 174, 175, and 176, respectively or of SEQ ID NOs: 184, 185, 186, 188, 189, 190, respectively.

In certain preferred embodiments, the disclosure relates to a monoclonal antibody or antigen-binding fragment thereof that specifically binds to anti-FXIa antibody 076D-M007-H04-CDRL3-N110D and thereby inhibits the neutralizing activity of this anti-FXIa antibody, wherein the antibody or antigen binding fragment thereof comprises a variable heavy chain (VH) sequence and a variable light chain (VL) sequence comprising the amino acid sequences of SEQ ID NOs: 141 and 145, or SEQ ID NOs: 169 and 173, or SEQ ID NOs: 183 and 187, respectively.

In certain especially preferred embodiments, the disclosure relates to a monoclonal antibody that specifically binds to anti-FXIa antibody 076D-M007-H04-CDRL3-N110D and thereby inhibits the neutralizing activity of this anti-FXIa antibody, wherein the antibody or antigen binding fragment thereof comprises the heavy chain sequence of SEQ ID NOs: 151 and the light chain sequence of SEQ ID NO: 152.

In some embodiments, the monoclonal antibody or antigen-binding fragment thereof that specifically binds to anti-FXIa antibody 076D-M007-H04-CDRL3-N110D and thereby inhibits the neutralizing activity of this anti-FXIa antibody is chimeric, humanized, or human.

An antibody of the invention may be an IgG (immunoglobulin G e.g. IgG1 IgG2, IgG3, IgG4) or IgA, IgD, IgE, IgM, or a derivative thereof, as a monovalent antibody, for example, while an antibody fragment may be a Fab, Fab′, F(ab′)₂, Fab′-SH or scFv, for example. An inventive antibody fragment, accordingly, may be, or may contain, an antigen-binding region that behaves in one or more ways as described herein.

In preferred embodiment, the antibody or antigen-binding fragment thereof that specifically binds to anti-FXIa antibody 076D-M007-H04-CDRL3-N110D and thereby inhibits the neutralizing activity of this anti-FXIa antibody comprises a human IgG heavy chain constant region.

In especially preferred embodiment, the antibody or antigen-binding fragment thereof that specifically binds to anti-FXIa antibody 076D-M007-H04-CDRL3-N110D and thereby inhibits the neutralizing activity of this anti-FXIa antibody comprises a human IgG1 heavy chain constant region.

In preferred embodiments, the antigen-binding antibody fragment that specifically binds to anti-FXIa antibody 076D-M007-H04-CDRL3-N110D and thereby inhibits the neutralizing activity of this anti-FXIa antibody is a Fab fragment.

In further especially preferred aspects, the disclosure relates to a Fab fragment that specifically binds to anti-FXIa antibody 076D-M007-H04-CDRL3-N110D and thereby inhibits the neutralizing activity of this anti-FXIa antibody, wherein the Fab fragment comprises the heavy chain sequence of SEQ ID NOs: 179 and the light chain sequence of SEQ ID NO: 180.

In further especially preferred aspects, the disclosure relates to a monovalent antibody that specifically binds to anti-FXIa antibody 076D-M007-H04-CDRL3-N110D and thereby inhibits the neutralizing activity of this anti-FXIa antibody, wherein the monovalent antibody comprises the heavy chain sequence of SEQ ID NOs: 191 and 193 and the light chain sequence of SEQ ID NO: 192.

In a preferred embodiment of the invention the antibodies or antigen-binding antibody fragments thereof are monoclonal.

In some embodiments, antibodies of the invention or antigen-binding fragments thereof or nucleic acids encoding the same are isolated. An isolated biological component (such as a nucleic acid molecule or protein such as an antibody) is one that has been substantially separated or purified away from other biological components in the cell of the organism in which the component naturally occurs, e.g., other chromosomal and extra-chromosomal DNA and RNA, proteins and organelles. The term also embraces nucleic acids and proteins prepared by recombinant expression in a host cell as well as chemically synthesized nucleic acids.

Antibody Generation

An antibody of the invention may be derived from a recombinant antibody library that is based on amino acid sequences that have been isolated from the antibodies of a large number of healthy volunteers e.g. using the n-CoDeR® technology the fully human CDRs are recombined into new antibody molecules (Carlson & Söderlind, Expert Rev Mol Diagn. 2001 May; 1(1):102-8). Or alternatively for example antibody libraries as the fully human antibody phage display library described in Hoet R M et al., Nat Biotechnol 2005; 23(3):344-8) can be used to isolate 076D-M007-H04-CDRL3-N110D-specific antibodies. Antibodies or antibody fragments isolated from human antibody libraries are considered human antibodies or human antibody fragments herein.

Human antibodies may be further prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. For example, immunization of genetically engineered mice inter alia immunization of hMAb mice (e.g. VelocImmune Mouse® or XENOMOUSE®) may be performed.

Further antibodies may be generated using the hybridoma technology (for example see Köhler and Milstein Nature. 1975 Aug. 7; 256(5517):495-7), resulting in for example murine, rat, or rabbit antibodies which can be converted into chimeric or humanized antibodies.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Natl Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall' Acqua et al., Methods 36:43-60 (2005) (describing “FR shuffling”); and Osboum et al., Methods 36:61-68 (2005) and Klimka et al., Br. J. Cancer, 83:252-260 (2000) (describing the “guided selection” approach to FR shuffling).

Examples are provided for the generation of antibodies using a recombinant antibody library and immunization of mice combined with subsequent humanization.

It is a further aspect of the invention to provide a method to generate antibodies specifically binding to anti-FXIa antibody 076D-M007-H04-CDRL3-N110D. It is an embodiment of the invention to provide a method for generation of anti-076D-M007-H04-CDRL3-N110D antibodies characterized by comprising the steps of immunization of an animal, determining the amino acid sequence of antibodies specifically binding to anti-FXIa antibody 076D-M007-H04-CDRL3-N110D, followed optionally by humanization or generation of a chimeric antibody, and expression of said antibodies. The expression system can be a recombinant or a cell free expression system. Suitable host cells for recombinant expression are prokaryotic and eukaryotic cells. Preferred are mammalian expression systems.

Peptide Variants

Antibodies or antigen-binding fragments of the invention are not limited to the specific peptide sequences provided herein. Rather, the invention also embodies variants of these polypeptides. With reference to the instant disclosure and conventionally available technologies and references, the skilled worker will be able to prepare, test and utilize functional variants of the antibodies disclosed herein, while appreciating these variants having the ability to bind to CEACAM6 fall within the scope of the present invention.

A variant can include, for example, an antibody that has at least one altered complementary determining region (CDR) (hyper-variable) and/or framework (FR) (variable) domain/position, vis-A-vis a peptide sequence disclosed herein.

By altering one or more amino acid residues in a CDR or FR region, the skilled worker routinely can generate mutated or diversified antibody sequences, which can be screened against the antigen, for new or improved properties, for example.

A further preferred embodiment of the invention is an antibody or antigen-binding fragment in which the VH and VL sequences are selected as shown in Table 1 and FIG. 7. The skilled worker can use the data in Table 1 or FIG. 7 to design peptide variants that are within the scope of the present invention. It is preferred that variants are constructed by changing amino acids within one or more CDR regions; a variant might also have one or more altered framework regions. Alterations also may be made in the framework regions. For example, a peptide FR domain might be altered where there is a deviation in a residue compared to a germline sequence.

Alternatively, the skilled worker could make the same analysis by comparing the amino acid sequences disclosed herein to known sequences of the same class of such antibodies, using, for example, the procedure described by Knappik A., et al., JMB 2000, 296:57-86.

Furthermore, variants may be obtained by using one antibody as starting point for further optimization by diversifying one or more amino acid residues in the antibody, preferably amino acid residues in one or more CDRs, and by screening the resulting collection of antibody variants for variants with improved properties. Particularly preferred is diversification of one or more amino acid residues in CDR3 of VL and/or VH. Diversification can be done e.g. by synthesizing a collection of DNA molecules using trinucleotide mutagenesis (TRIM) technology (Virnekas B. et al., Nucl. Acids Res. 1994, 22: 5600.). Antibodies or antigen-binding fragments thereof include molecules with modifications/variations including but not limited to e.g. modifications leading to altered half-life (e.g. modification of the Fc part or attachment of further molecules such as PEG), altered binding affinity or altered ADCC or CDC activity.

Conservative Amino Acid Variants

Polypeptide variants may be made that conserve the overall molecular structure of an antibody peptide sequence described herein. Given the properties of the individual amino acids, some rational substitutions will be recognized by the skilled worker. Amino acid substitutions, i.e., “conservative substitutions,” may be made, for instance, on the basis of similarity in polarity, charge, solubility, hydrophobicity, hydrophilicity, and/or the amphipathic nature of the residues involved.

For example, (a) nonpolar (hydrophobic) amino acids include alanine, leucine, isoleucine, valine, proline, phenylalanine, tryptophane, and methionine; (b) polar neutral amino acids include glycine, serine, threonine, cysteine, tyrosine, asparagine, and glutamine; (c) positively charged (basic) amino acids include arginine, lysine, and histidine; and (d) negatively charged (acidic) amino acids include aspartic acid and glutamic acid. Substitutions typically may be made within groups (a)-(d). In addition, glycine and proline may be substituted for one another based on their ability to disrupt α-helices. Similarly, certain amino acids, such as alanine, cysteine, leucine, methionine, glutamic acid, glutamine, histidine and lysine are more commonly found in α-helices, while valine, isoleucine, phenylalanine, tyrosine, tryptophan and threonine are more commonly found in β-pleated sheets. Glycine, serine, aspartic acid, asparagine, and proline are commonly found in turns. Some preferred substitutions may be made among the following groups: (i) S and T; (ii) P and G; and (iii) A, V, L and I. Given the known genetic code, and recombinant and synthetic DNA techniques, the skilled scientist readily can construct DNAs encoding the conservative amino acid variants.

Glycosylation Variants

Where the antibody comprises an Fe region, the carbohydrate attached thereto may be altered. Native antibodies produced by mammalian cells typically comprise a branched, biantennary oligosaccharide that is generally attached by an N-linkage to Asn297 using Kabat EU numbering of the CH2 domain of the Fc region; see, e.g., Wright et al. Trends Biotechnol. 15: 26-32 (1997).

In certain embodiments, an antibody provided herein is altered to increase or decrease the extent to which the antibody is glycosylated. Addition or deletion of glycosylation sites to an antibody may be conveniently accomplished by altering the expression system (e.g. host cell) and/or by altering the amino acid sequence such that one or more glycosylation sites is created or removed.

In one embodiment of this invention, aglycosyl antibodies having decreased effector function or antibody derivatives are prepared by expression in a prokaryotic host. Suitable prokaryotic hosts for include but are not limited to E. coli, Bacillus subtilis, Salmonella typhimurium and various species within the genera Pseudomonas, Streptomyces, and Staphylococcus.

In one embodiment, antibody variants are provided having decreased effector function, which are characterized by a modification at the conserved N-linked site in the CH2 domains of the Fc portion of said antibody. In one embodiment of present invention, the modification comprises a mutation at the heavy chain glycosylation site to prevent glycosylation at the site.

Thus, in one preferred embodiment of this invention, the aglycosyl antibodies or antibody derivatives are prepared by mutation of the heavy chain glycosylation site,—i.e., mutation of N297 using Kabat EU numbering and expressed in an appropriate host cell.

In another embodiment of the present invention, aglycosyl antibodies or antibody derivatives have decreased effector function, wherein the modification at the conserved N-linked site in the CH2 domains of the Fc portion of said antibody or antibody derivative comprises the removal of the CH2 domain glycans,—i.e., deglycosylation. These aglycosyl antibodies may be generated by conventional methods and then deglycosylated enzymatically. Methods for enzymatic deglycosylation of antibodies are well known in the art (e.g. Winkelhake & Nicolson (1976), J Biol Chem. 251(4):1074-80).

In another embodiment of this invention, deglycosylation may be achieved using the glycosylation inhibitor tunicamycin (Nose & Wigzell (1983), Proc Natl Acad Sci USA, 80(21):6632-6). That is, the modification is the prevention of glycosylation at the conserved N-linked site in the CH2 domains of the Fc portion of said antibody.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function.

Examples of publications related to “defucosylated” or “fucose-deficient” antibody variants include: Okazaki et al. J Mol. Biol. 336: 1239-1249 (2004); Yamane-Ohnuki et al. Biotech. Bioeng. 87: 614 (2004).

Examples of cell lines capable of producing defucosylated antibodies include Lec13 CHO cells deficient in protein fucosylation (Ripka et al. Arch. Biochem. Biophys. 249:533-545 (1986); and WO 2004/056312), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006)).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878; U.S. Pat. No. 6,602,684; and US 2005/0123546.

Antibody variants with at least one galactose residue in the oligosaccharide attached to the Fc region are also provided. Such antibody variants may have improved CDC function. Such antibody variants are described, e.g., in WO1997/30087; WO1998/58964; and WO1999/22764.

Fc Region Variants

In certain embodiments, one or more amino acid modifications (e.g. a substitution) may be introduced into the Fc region of an antibody (e.g., a human IgG1, IgG2, IgG3 or IgG4 Fc region) provided herein, thereby generating an Fc region variant.

In certain embodiments, the invention contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity) but retains FcRn binding ability. In some embodiments, alterations are made in the Fc region that result in altered (i.e., either improved or diminished) C1q binding and/or Complement Dependent Cytotoxicity (CDC).

In certain embodiments, the invention contemplates an antibody variant that possesses an increased or decreased half-live. Antibodies with increased half-lives and improved binding to the neonatal Fc receptor (FcRn), which is responsible for the transfer of maternal IgGs to the fetus (Guyer et al., J Immunol. 117:587 (1976) and Kim et al., J Immunol. 24:249 (1994)), are described in US2005/0014934 (Hinton et al.). Those antibodies comprise an Fc region with one or more substitutions therein which improve binding of the Fc region to FcRn.

DNA Molecules of the Invention

The present invention also relates to the DNA molecules that encode an antibody of the invention or antigen-binding fragment thereof. The DNA sequences used for the antibodies expressed are given in FIG. 7. These sequences are optimized in certain cases for mammalian expression. DNA molecules of the invention are not limited to the sequences disclosed herein, but also include variants thereof. DNA variants within the invention may be described by reference to their physical properties in hybridization. The skilled worker will recognize that DNA can be used to identify its complement and, since DNA is double stranded, its equivalent or homolog, using nucleic acid hybridization techniques. It also will be recognized that hybridization can occur with less than 100% complementarity. However, given appropriate choice of conditions, hybridization techniques can be used to differentiate among DNA sequences based on their structural relatedness to a particular probe. For guidance regarding such conditions see, Sambrook et al., 1989 supra and Ausubel et al., 1995 (Ausubel, F. M., Brent, R., Kingston, R. E., Moore, D. D., Sedman, J. G., Smith, J. A., & Struhl, K. eds. (1995). Current Protocols in Molecular Biology. New York: John Wiley and Sons).

Structural similarity between two polynucleotide sequences can be expressed as a function of “stringency” of the conditions under which the two sequences will hybridize with one another. As used herein, the term “stringency” refers to the extent that the conditions disfavor hybridization. Stringent conditions strongly disfavor hybridization, and only the most structurally related molecules will hybridize to one another under such conditions. Conversely, non-stringent conditions favor hybridization of molecules displaying a lesser degree of structural relatedness. Hybridization stringency, therefore, directly correlates with the structural relationships of two nucleic acid sequences.

Hybridization stringency is a function of many factors, including overall DNA concentration, ionic strength, temperature, probe size and the presence of agents which disrupt hydrogen bonding. Factors promoting hybridization include high DNA concentrations, high ionic strengths, low temperatures, longer probe size and the absence of agents that disrupt hydrogen bonding. Hybridization typically is performed in two phases: the “binding” phase and the “washing” phase.

Functionally Equivalent DNA Variants

Yet another class of DNA variants within the scope of the invention may be described with reference to the product they encode. These functionally equivalent polynucleotides are characterized by the fact that they encode the same peptide sequences due to the degeneracy of the genetic code.

It is recognized that variants of DNA molecules provided herein can be constructed in several different ways. For example, they may be constructed as completely synthetic DNAs. Methods of efficiently synthesizing oligonucleotides are widely available. See Ausubel et al., section 2.11, Supplement 21 (1993). Overlapping oligonucleotides may be synthesized and assembled in a fashion first reported by Khorana et al., J. Mol. Biol. 72:209-217 (1971); see also Ausubel et al., supra, Section 8.2. Synthetic DNAs preferably are designed with convenient restriction sites engineered at the 5′ and 3′ ends of the gene to facilitate cloning into an appropriate vector.

As indicated, a method of generating variants is to start with one of the DNAs disclosed herein and then to conduct site-directed mutagenesis. See Ausubel et al., supra, chapter 8, Supplement 37 (1997). In a typical method, a target DNA is cloned into a single-stranded DNA bacteriophage vehicle. Single-stranded DNA is isolated and hybridized with an oligonucleotide containing the desired nucleotide alteration(s). The complementary strand is synthesized and the double stranded phage is introduced into a host. Some of the resulting progeny will contain the desired mutant, which can be confirmed using DNA sequencing. In addition, various methods are available that increase the probability that the progeny phage will be the desired mutant. These methods are well known to those in the field and kits are commercially available for generating such mutants.

Recombinant DNA Constructs and Expression

The present invention further provides recombinant DNA constructs comprising one or more of the nucleotide sequences of the present invention. The recombinant constructs of the present invention can be used in connection with a vector, such as a plasmid, phagemid, phage or viral vector, into which a DNA molecule encoding an antibody of the invention or antigen-binding fragment thereof or variant thereof is inserted.

An antibody, antigen binding portion, or variant thereof provided herein can be prepared by recombinant expression of nucleic acid sequences encoding light and heavy chains or portions thereof in a host cell. To express an antibody, antigen binding portion, or variant thereof recombinantly a host cell can be transfected with one or more recombinant expression vectors carrying DNA fragments encoding the light and/or heavy chains or portions thereof such that the light and heavy chains are expressed in the host cell. Standard recombinant DNA methodologies are used to prepare and/or obtain nucleic acids encoding the heavy and light chains, incorporate these nucleic acids into recombinant expression vectors and introduce the vectors into host cells, such as those described in Sambrook, Fritsch and Maniatis (eds.), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397 by Boss et al.

In addition, the nucleic acid sequences encoding variable regions of the heavy and/or light chains can be converted, for example, to nucleic acid sequences encoding full-length antibody chains, Fab fragments, or to scFv. The VL- or VH-encoding DNA fragment can be operatively linked, (such that the amino acid sequences encoded by the two DNA fragments are in-frame) to another DNA fragment encoding, for example, an antibody constant region or a flexible linker. The sequences of human heavy chain and light chain constant regions are known in the art (see e.g., Kabat, E. A., el al. (1991) Sequences of Proteins of Immunological Interest, Fifth Edition, U.S. Department of Health and Human Services, NIH Publication No. 91-3242) and DNA fragments encompassing these regions can be obtained by standard PCR amplification.

To create a polynucleotide sequence that encodes a scFv, the VH- and VL-encoding nucleic acids can be operatively linked to another fragment encoding a flexible linker such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., Nature (1990) 348:552-554).

To express the antibodies, antigen binding fragments thereof or variants thereof standard recombinant DNA expression methods can be used (see, for example, Goeddel; Gene Expression Technology. Methods in Enzymology 185, Academic Press, San Diego, Calif. (1990)). For example, DNA encoding the desired polypeptide can be inserted into an expression vector which is then transfected into a suitable host cell. Suitable host cells are prokaryotic and eukaryotic cells. Examples for prokaryotic host cells are e.g. bacteria, examples for eukaryotic hosts cells are yeasts, insects and insect cells, plants and plant cells, transgenic animals, or mammalian cells. In some embodiments, the DNAs encoding the heavy and light chains are inserted into separate vectors. In other embodiments, the DNA encoding the heavy and light chains is inserted into the same vector. It is understood that the design of the expression vector, including the selection of regulatory sequences is affected by factors such as the choice of the host cell, the level of expression of protein desired and whether expression is constitutive or inducible.

Therefore, an embodiment of the present invention are also host cells comprising the vector or a nucleic acid molecule, whereby the host cell can be a higher eukaryotic host cell, such as a mammalian cell, a lower eukaryotic host cell, such as a yeast cell, and may be a prokaryotic cell, such as a bacterial cell.

Another embodiment of the present invention is a method of using the host cell to produce an antibody and antigen binding fragments, comprising culturing the host cell under suitable conditions and recovering said antibody.

Therefore, another embodiment of the present invention is the production of the antibodies according to this invention with the host cells of the present invention and purification of these antibodies to at least 95% homogeneity by weight.

Bacterial Expression

Useful expression vectors for bacterial use are constructed by inserting a DNA sequence encoding a desired protein together with suitable translation initiation and termination signals in operable reading phase with a functional promoter. The vector will comprise one or more phenotypic selectable markers and an origin of replication to ensure maintenance of the vector and, if desirable, to provide amplification within the host. Suitable prokaryotic hosts for transformation include but are not limited to E. coli, Bacillus subtilis, Salmonella typhimurium and various species within the genera Pseudomonas, Streptomyces, and Staphylococcus.

Bacterial vectors may be, for example, bacteriophage-, plasmid- or phagemid-based. These vectors can contain a selectable marker and a bacterial origin of replication derived from commercially available plasmids typically containing elements of the well-known cloning vector pBR322 (ATCC 37017). Following transformation of a suitable host strain and growth of the host strain to an appropriate cell density, the selected promoter is de-repressed/induced by appropriate means (e.g., temperature shift or chemical induction) and cells are cultured for an additional period. Cells are typically harvested by centrifugation, disrupted by physical or chemical means, and the resulting crude extract retained for further purification.

In bacterial systems, a number of expression vectors may be advantageously selected depending upon the use intended for the protein being expressed. For example, when a large quantity of such a protein is to be produced, for the generation of antibodies or to screen peptide libraries, for example, vectors which direct the expression of high levels of fusion protein products that are readily purified may be desirable.

Therefore, an embodiment of the present invention is an expression vector comprising a nucleic acid sequence encoding for the novel antibodies of the present invention.

Antibodies of the present invention or antigen-binding fragments thereof or variants thereof include naturally purified products, products of chemical synthetic procedures, and products produced by recombinant techniques from a prokaryotic host, including, for example, E. coli, Bacillus subtilis, Salmonella typhimurium and various species within the genera Pseudomonas, Streptomyces, and Staphylococcus, preferably, from E. coli cells.

Mammalian Expression

Preferred regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. Expression of the antibodies may be constitutive or regulated (e.g. inducible by addition or removal of small molecule inductors such as Tetracyclin in conjunction with Tet system). For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al. The recombinant expression vectors can also include origins of replication and selectable markers (see e.g., U.S. Pat. Nos. 4,399,216, 4,634,665 and 5,179,017). Suitable selectable markers include genes that confer resistance to drugs such as G418, puromycin, hygromycin, blasticidin, zeocin/bleomycin or methotrexate or selectable marker that exploit auxotrophies such as Glutamine Synthetase (Bebbington et al., Biotechnology (N Y). 1992 February; 10(2):169-75), on a host cell into which the vector has been introduced. For example, the dihydrofolate reductase (DHFR) gene confers resistance to methotrexate, neo gene confers resistance to G418, the bsd gene from Aspergillus terreus confers resistance to blasticidin, puromycin N-acetyl-transferase confers resistance to puromycin, the Sh ble gene product confers resistance to zeocin, and resistance to hygromycin is conferred by the E. coli hygromycin resistance gene (hyg or hph). Selectable markers like DHFR or Glutamine Synthetase are also useful for amplification techniques in conjunction with MTX and MSX.

Transfection of the expression vector into a host cell can be carried out using standard techniques such as electroporation, nucleofection, calcium-phosphate precipitation, lipofection, polycation-based transfection such as polyethlylenimine (PEI)-based transfection and DEAE-dextran transfection.

Suitable mammalian host cells for expressing the antibodies, antigen binding fragments thereof or variants thereof provided herein include Chinese Hamster Ovary (CHO cells) such as CHO-K1, CHO-S, CHO-KISV [including dhfr-CHO cells, described in Urlaub and Chasin, (1980) Proc. Natl. Acad. Sci. USA 77:4216-4220 and Urlaub et al., Cell. 1983 June; 33(2):405-12, used with a DHFR selectable marker, e.g., as described in R. J. Kaufman and P. A. Sharp (1982) Mol. Biol. 159:601-621; and other knockout cells exemplified in Fan et al., Biotechnol Bioeng. 2012 April; 109(4):1007-15], NS0 myeloma cells, COS cells, HEK293 cells, HKB11 cells, BHK21 cells, CAP cells, EB66 cells, and SP2 cells.

Expression might also be transient or semi-stable in expression systems such as HEK293, HEK293T, HEK293-EBNA, HEK293E, HEK293-6E, HEK293-Freestyle, HKB11, Expi293F, 293EBNALT75, CHO Freestyle, CHO-S, CHO-K1, CHO-KISV, CHOEBNALT85, CHOS-XE, CHO-3E7 or CAP-T cells (for instance Durocher et al., Nucleic Acids Res. 2002 Jan. 15; 30(2):E9).

In some embodiments, the expression vector is designed such that the expressed protein is secreted into the culture medium in which the host cells are grown. The antibodies, antigen binding fragments thereof or variants thereof can be recovered from the culture medium using standard protein purification methods.

Purification

Antibodies of the invention or antigen-binding fragments thereof or variants thereof can be recovered and purified from recombinant cell cultures by well-known methods including, but not limited to ammonium sulfate or ethanol precipitation, acid extraction, Protein A chromatography, Protein G chromatography, anion or cation exchange chromatography, phospho-cellulose chromatography, hydrophobic interaction chromatography, affinity chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

Antibodies of the present invention or antigen-binding fragments thereof or variants thereof include naturally purified products, products of chemical synthetic procedures, and products produced by recombinant techniques from an eukaryotic host, including, for example, yeast, higher plant, insect and mammalian cells. Depending upon the host employed in a recombinant production procedure, the antibody of the present invention can be glycosylated or can be non-glycosylated. Such methods are described in many standard laboratory manuals, such as Sambrook, supra, Sections 17.37-17.42; Ausubel, supra, Chapters 10, 12, 13, 16, 18 and 20.

In preferred embodiments, the antibody is purified (1) to greater than 95% by weight of antibody as determined e.g. by the Lowry method, UV-Vis spectroscopy or by SDS-Capillary Gel electrophoresis (for example on a Caliper LabChip GXII, GX 90 or Biorad Bioanalyzer device), and in further preferred embodiments more than 99% by weight, (2) to a degree sufficient to obtain at least 15 residues of N-terminal or internal amino acid sequence, or (3) to homogeneity by SDS-PAGE under reducing or non-reducing conditions using Coomassie blue or, preferably, silver stain. Isolated naturally occurring antibody includes the antibody in situ within recombinant cells since at least one component of the antibody's natural environment will not be present. Ordinarily, however, isolated antibody will be prepared by at least one purification step.

Pharmaceutical Compositions and Administration

Pharmaceutical compositions for use in accordance with the present invention may be formulated in a conventional manner using one or more physiologically acceptable carriers or excipients. An antibody of the invention or antigen-binding fragment thereof can be administered by any suitable means. Possible administration routes include parenteral (e.g., intramuscular, intravenous, intra-arterial, intraperitoneal, or subcutaneous), intrapulmonary and intranasal, and, if desired for local immunosuppressive treatment, intralesional administration. In addition, an antibody of the invention or an antigen-binding fragment thereof or a variant thereof might be administered by pulse infusion, with, e.g., declining doses of the antibody. Preferably, the dosing is given by injections, most preferably intravenous or subcutaneous injections, depending in part on whether the administration is brief or chronic. The amount to be administered will depend on a variety of factors such as the clinical symptoms, weight of the individual, whether other drugs are administered.

An embodiment of the present invention are pharmaceutical compositions which comprise anti-076D-M007-H04-CDRL3-N110D antibodies or antigen-binding fragments thereof (such as Fab fragments), or variants thereof, alone or in combination with at least one other agent, such as a stabilizing compound, which may be administered in any sterile, biocompatible pharmaceutical carrier, including, but not limited to, saline, buffered saline, dextrose, and water. A further embodiment are pharmaceutical compositions comprising a 076D-M007-H04-CDRL3-N110D binding antibody or antigen-binding fragment thereof and a further pharmaceutically active compound that is suitable to treat FXI/a related diseases. Any of these molecules can be administered to a patient alone, or in combination with other agents, drugs or hormones, in pharmaceutical compositions where it is mixed with excipient(s) or pharmaceutically acceptable carriers. In one embodiment of the present invention, the pharmaceutically acceptable carrier is pharmaceutically inert.

The present invention also relates to the administration of pharmaceutical compositions. Such administration is accomplished orally or parenterally. Methods of parenteral delivery include topical, intra-arterial (directly to the tumor), intramuscular, subcutaneous, intramedullary, intrathecal, intraventricular, intravenous, intraperitoneal, or intranasal administration. In addition to the active ingredients, these pharmaceutical compositions may contain suitable pharmaceutically acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Ed. Maack Publishing Co, Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, or other plants; cellulose such as methyl-cellulose, hydroxypropylmethylcellulose, or sodium carboxymethyl cellulose; and gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores can be provided with suitable coatings such as concentrated sugar solutions, which may also contain gum arabic, talc, polyvinyl pyrrolidone, carbopol gel, polyethylene glycol and/or titanium dioxide, lacquer solutions, and suitable organic solvents or solvent mixtures. Dyestuffs or pigments may be added to the tablets or dragee coatings for product identification or to characterize the quantity of active compound, i.e. dosage.

Pharmaceutical preparations that can be used orally include push-fit capsules made of gelatin, as well as soft, sealed capsules made of gelatin and a coating such as glycerol or sorbitol. Push-fit capsules can contain active ingredients mixed with a filler or binders such as lactose or starches, lubricants such as talc or magnesium stearate, and optionally, stabilizers. In soft capsules, the active compounds may be dissolved or suspended in suitable liquids, such as fatty oils, liquid paraffin, or liquid polyethylene glycol with or without stabilizers.

Pharmaceutical formulations for parenteral administration include aqueous solutions of active compounds. For injection, the pharmaceutical compositions of the invention may be formulated in aqueous solutions, preferably in physiologically compatible buffers such as Hank's solution, Ringer's solution, or physiologically buffered saline. Aqueous injection suspensions may contain substances that increase viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

For topical or nasal administration, penetrants appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping or lyophilizing processes.

The pharmaceutical composition may be provided as a salt and can be formed with acids, including but not limited to hydrochloric, sulfuric, acetic, lactic, tartaric, malic, succinic, etc. Salts tend to be more soluble in aqueous or other protonic solvents that are the corresponding free base forms. In other cases, the preferred preparation may be a lyophilized powder in 1 mM-50 mM histidine or phosphate or Tris, 0.1%-2% sucrose and/or 2%-7% mannitol at a pH range of 4.5 to 7.5 optionally comprising additional substances like polysorbate that is combined with buffer prior to use.

After pharmaceutical compositions comprising a compound of the invention formulated in an acceptable carrier have been prepared, they can be placed in an appropriate container and labeled for treatment of an indicated condition. For administration of anti-076D-M007-H04-CDRL3-N110D antibodies or antigen-binding fragment thereof, such as Fab fragments, such labeling would include amount, frequency and method of administration.

Kits

The invention further relates to pharmaceutical packs and kits comprising one or more containers filled with one or more of the ingredients of the aforementioned compositions of the invention. Associated with such container(s) can be a notice in the form prescribed by a governmental agency regulating the manufacture, use or sale of pharmaceuticals or biological products, reflecting approval by the agency of the manufacture, use or sale of the product for human administration.

Prophylactic and Therapeutic Uses

The present disclosure relates to methods for neutralizing (e.g., partially neutralizing) the therapeutic activity of an anti-FXIa antibody in a patient being treated with the anti-FXIa antibody or antigen-binding fragment thereof, comprising administering an effective amount of a reversal agent provided herein, e.g., a reversal agent (e.g., antibody or antigen-binding fragment thereof, such as a Fab fragment) which binds an anti-FXIa antibody and is capable of neutralizing its therapeutic activity. In specific aspects, neutralizing the therapeutic activity of an anti-FXIa antibody may be needed by a patient for emergency surgery/urgent procedures and in life-threatening or uncontrolled bleeding. In particular aspects, a patient is being treated with an anti-FXI/FXIa antibody to manage, treat, prevent, or reduce the risk of a thromboembolic disease or disorder, for example reducing the risk of stroke or thrombosis (e.g., systemic embolism) in patients with atrial fibrillation (e.g., non-valvular atrial fibrillation), chronic kidney disease, such as end stage renal failure (ESRD) undergoing hemodialysis or following surgery (e.g. orthopaedic surgery). In further specific aspects, the patient has a demonstrated high risk of bleeding. In specific aspects, non-limiting examples of anti-FXIa antibody reversal agents for use in these methods include antibodies and antigen-binding fragments, such as Fab fragments, described herein, e.g., in Table 1, for example, antibodies “TPP-8236”, “TPP-8237”, “TPP-8238”, “TPP-8239”, “TPP-8240”, “TPP-8241”, “TPP-8343”, “TPP-8246”, “TPP-9238”, “TPP-9251”, “TPP-9252”, “TPP-9258”, monovalent antibody “TPP-20816” or Fab fragment “TPP-10089”; antibodies comprising VH CDRs and VL CDRs of such antibodies; antibodies that bind the same epitope(s) within target antibody anti-FXIa antibody 076D-M007-H04-CDRL3-N110D as such antibodies.

In specific aspects, the present disclosure relates to methods for neutralizing (e.g., partially neutralizing) the therapeutic activity of anti-FXIa antibody 076D-M007-H04-CDRL3-N110D, and to related methods as essential part of a general bleeding management in a patient being treated with this anti-FXIa antibody comprising administering an effective amount of a reversal agent provided herein, e.g., a reversal agent (e.g., antibody or antigen-binding fragment thereof, such as a Fab fragment) which binds anti-FXIa antibody 076D-M007-H04-CDRL3-N110D and is capable of neutralizing its therapeutic activity. In specific aspects, neutralization of the therapeutic activity of anti-FXIa antibody 076D-M007-H04-CDRL3-N110D may be needed by a patient for emergency surgery/urgent procedures and in life-threatening or uncontrolled bleeding. In particular aspects, a patient is being treated with the anti-FXIa antibody 076D-M007-H04-CDRL3-N110D to manage, treat, prevent, or reduce the risk of a thromboembolic disease or disorder, for example reducing the risk of stroke or thrombosis (e.g., systemic embolism) in patients with atrial fibrillation (e.g., non-valvular atrial fibrillation), chronic kidney disease, such as end stage renal failure (ESRD) undergoing hemodialysis, or following surgery (e.g. orthopaedic surgery). In further specific aspects, the patient has a demonstrated high risk of bleeding. In specific aspects, non-limiting examples of anti-FXIa antibody reversal agents for use in these methods include antibodies and antigen-binding fragments, such as Fab fragments, described herein, e.g., in Table 1, for example, antibodies “TPP-8236”, “TPP-8237”, “TPP-8238”, “TPP-8239”, “TPP-8240”, “TPP-8241”, “TPP-8343”, “TPP-8246”, “TPP-9238”, “TPP-9251”, “TPP-9252”, “TPP-9258”, monovalent antibody “TPP-20816” or Fab fragment “TPP-10089”; antibodies comprising VH CDRs and VL CDRs of such antibodies; antibodies that bind the same epitope(s) within target antibody anti-FXIa antibody 076D-M007-H04-CDRL3-N110D as such antibodies.

In a particular aspect, provided herein are methods for neutralizing the therapeutic activity an anti-FXIa antibody, and related methods as essential part of a general bleeding management in a patient treated or administered an anti-FXIa antibody as described in WO2013/167669, preferentially anti-FXIa antibody 076D-M007-H04-CDRL3-N110D, comprising the step of administering to the patient in need thereof, a reversal agent according to this invention, wherein the reversal agent specifically binds to the anti-FXIa antibody 076D-M007-H04-CDRL3-N110D and blocks the anti-FXIa antibody from binding to FXIa or reduces binding of the anti-FXIa antibody to FXIa. In specific embodiments, a reversal agent according to this invention neutralizes the therapeutic activity of an anti-FXIa antibody as described in WO2013/167669, preferentially anti-FXIa antibody 076D-M007-H04-CDRL3-N110D to mitigate bleeding risks, for example during urgent major surgery or trauma or to manage, treat, prevent, or reduce the risk of a thromboembolic disease or disorder, for example reducing the risk of stroke or thrombosis (e.g., systemic embolism) in patients with atrial fibrillation (e.g., non-valvular atrial fibrillation), chronic kidney disease, such as end stage renal failure (ESRD) undergoing hemodialysis, or following surgery (e.g. orthopaedic surgery).

In specific aspects, a reversal agent according to this invention neutralizes the therapeutic activity of an anti-FXIa antibody. In particular aspects, the reversal agent is administered to a patient in need thereof to temporarily neutralize the therapeutic activity of an anti-FXIa antibody as described in WO2013/167669, preferentially anti-FXIa antibody 076D-M007-H04-CDRL3-N110D.

In a particular aspect, provided herein are methods for neutralizing the therapeutic activity an anti-FXIa antibody, and to related methods as essential part of a general bleeding management in a patient treated or administered an anti-FXIa antibody as described in WO2013/167669, preferentially anti-FXIa antibody 076D-M007-H04-CDRL3-N110D, comprising the step of administering to the patient in need thereof, a reversal agent according to this invention, wherein the reversal agent specifically binds to the anti-FXIa antibody 076D-M007-H04-CDRL3-N110D and blocks the anti-FXIa antibody from binding to FXIa or reduces binding of the anti-FXIa antibody to FXIa. In a specific embodiment, the reversal agent according to this invention neutralizes the therapeutic activity of the anti-FXIa antibody 076D-M007-H04-CDRL3-N110D. In certain embodiments, a temporary neutralization of the therapeutic activity of the anti-FXIa antibody 076D-M007-H04-CDRL3-N110D is achieved. In specific embodiments, following the temporary neutralization of the anti-FXIa antibody 076D-M007-H04-CDRL3-N110D, the anti-FXIa antibody 076D-M007-H04-CDRL3-N110D is again administered to the patient.

As used herein, the terms “effective amount” or “therapeutically effective amount” refer to an amount of a therapy (e.g., a reversal agent provided herein such as antibody that binds an anti-FXIa antibody, preferentially anti-FXIa antibody 076D-M007-H04-CDRL3-N110D, or a pharmaceutical composition provided herein) which is sufficient to reduce and/or ameliorate the severity and/or duration of a given condition, disorder, or disease and/or a symptom related thereto. These terms also encompass an amount necessary for the reduction, slowing, or amelioration of the advancement or progression of a given condition, disorder, or disease, reduction, slowing, or amelioration of the recurrence, development or onset of a given condition, disorder or disease, and/or to improve or enhance the prophylactic or therapeutic effect(s) of another therapy (e.g., a therapy other than an anti-FXIa antibody reversal agent provided herein). In some aspects, “effective amount” as used herein also refers to the amount of an antibody described herein to achieve a specified result, for example, neutralization of the therapeutic activity (e.g., aPTT prolongation, and reduction in the amount of thrombin in a thrombin generation assay (TGA) in human plasma) of a target anti-FXIa antibody; and reduction in, or blocking, binding of a target anti-FXIa antibody to FXIa.

Determining a therapeutically effective amount of the reversal agents of this invention largely will depend on particular patient characteristics, route of administration, and the nature of the disorder being treated. General guidance can be found, for example, in the publications of the International Conference on Harmonization and in REMINGTON'S PHARMACEUTICAL SCIENCES, chapters 27 and 28, pp. 484-528 (18th ed., Alfonso R. Gennaro, Ed., Easton, Pa.: Mack Pub. Co., 1990). More specifically, determining a therapeutically effective amount will depend on such factors as toxicity and efficacy of the medicament. Toxicity may be determined using methods well known in the art and found in the foregoing references. Efficacy may be determined utilizing the same guidance in conjunction with the methods described below in the Examples.

For any compound, the therapeutically effective dose can be estimated initially either in cell culture assays, e.g., neoplastic cells, or in animal models, usually mice, rabbits, dogs, pigs or monkeys. The animal model is also used to achieve a desirable concentration range and route of administration. Such information can then be used to determine useful doses and routes for administration in humans.

Therapeutic efficacy and toxicity of a compound can be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, ED₅₀/LD₅₀. Pharmaceutical compositions that exhibit large therapeutic indices are preferred. The data obtained from cell culture assays and animal studies are used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The exact dosage is chosen by the individual physician in view of the patient to be treated. Dosage and administration are adjusted to provide sufficient levels of the active moiety or to maintain the desired effect. Additional factors that may be taken into account include the severity of the disease state; age, weight and gender of the patient; diet, time and frequency of administration, drug combination(s), reaction sensitivities, and tolerance/response to therapy. Long acting pharmaceutical compositions might be administered for example every 3 to 4 days, every week, once every two weeks, once every three weeks, once every 4 weeks, once every two month or once every three month depending on half-life and clearance rate of the particular formulation.

Normal dosage amounts may vary from 0.1 to 100,000 micrograms, up to a total dose of about 10 g, depending upon the route of administration. Guidance as to particular dosages and methods of delivery is provided in the literature. See U.S. Pat. Nos. 4,657,760; 5,206,344; or 5,225,212. Those skilled in the art will employ different formulations for polynucleotides than for proteins or their inhibitors. Similarly, delivery of polynucleotides or polypeptides will be specific to particular cells, conditions, locations, etc. Preferred specific activities for a radiolabelled antibody may range from 0.1 to 10 mCi/mg of protein (Riva et al., Clin. Cancer Res. 5:3275-3280, 1999; Ulaner et al., 2008 Radiology 246(3):895-902).

In specific aspects, a patient, who may be in need of, or may benefit from, the methods described herein (e.g., methods for neutralizing the therapeutic activity of an anti-FXIa antibody with anti-FXIa antibody reversal agents), has been treated with an anti-FXIa antibody, e.g. anti-FXIa antibody 076D-M007-H04-CDRL3-N110D, to manage, treat, prevent, or reduce the risk of a thromboembolic disease or disorder, e.g., thrombic stroke, atrial fibrillation, stroke prevention in atrial fibrillation (SPAF), deep vein thrombosis, venous thromboembolism (VTE), pulmonary embolism (PE), acute coronary syndromes (ACS), ischemic stroke, acute limb ischemia, chronic thromboembolic pulmonary hypertension, systemic embolism, or atherothrombosis. In further specific aspects, the patient has a demonstrated high risk of bleeding.

In other aspects, a patient, who may be in need of, or may benefit from, the methods described herein (e.g., methods for neutralizing the therapeutic activity of an anti-FXIa antibody with anti-FXIa antibody reversal agents), has been treated with an anti-FXIa antibody (e.g. anti-FXIa antibody 076D-M007-H04-CDRL3-N110D) for treatment and/or prophylaxis of FXI/FXIa related disorders, in particular cardiovascular disorders, preferably thrombotic or thromboembolic disorders and/or thrombotic or thromboembolic complications such as acute VTE, primary and extended secondary prevention of VTE, prevention of major adverse thromboembolic events in patient undergoing dialysis (with or without AF), prevention of major cardiovascular and cerebral events (MACCE) in patients with CAD undergoing PCI and receiving single or dual antiplatelet therapy, post-acute coronary syndromes (ACS) patients, heparin induced thrombocytopenia (HIT), prevention of thromboembolic events in heart failure patients and secondary stroke prevention.

For the purpose of the present invention, the “thrombotic or thromboembolic disorders” include disorders which occur both in the arterial and in the venous vasculature and which can be treated with the binding molecules of the invention, preferably antibodies and antigen-binding fragments thereof, in particular disorders in the coronary arteries of the heart, such as acute coronary syndrome (ACS), myocardial infarction with ST segment elevation (STEMI) and without ST segment elevation (non-STEMI), stable angina pectoris, unstable angina pectoris, reocclusions and restenoses after coronary interventions such as angioplasty, stent implantation or aortocoronary bypass, but also thrombotic or thromboembolic disorders in further vessels leading to peripheral arterial occlusive disorders, pulmonary embolisms, venous thromboembolisms, venous thromboses, in particular in deep leg veins and kidney veins, transitory ischaemic attacks and also thrombotic stroke and thromboembolic stroke.

In the context of the present invention, the term “pulmonary hypertension” includes pulmonary arterial hypertension, pulmonary hypertension associated with disorders of the left heart, pulmonary hypertension associated with pulmonary disorders and/or hypoxia and pulmonary hypertension owing to chronic thromboembolisms (CTEPH).

In specific aspects, a subject, who may be in need of, or benefit from, the methods described herein (e.g., methods for neutralizing the therapeutic activity of an anti-FXIa antibody with FXIa antibody reversal agents), has been treated with an anti-FXIa antibody (e.g., anti-FXIa antibody 076D-M007-H04-CDRL3-N110D) to manage, treat, prevent, or reduce the risk of one of the following conditions:
thromboembolism in subjects with suspected or confirmed cardiac arrhythmia such as paroxysmal, persistent or permanent atrial fibrillation or atrial flutter;
stroke prevention in atrial fibrillation (SPAF), a subpopulation of which is AF patients undergoing percutaneous coronary interventions (PCI);
acute venous thromboembolic events (VTE) treatment and extended secondary VTE prevention in patients at high risk for bleeding;
cerebral and cardiovascular events in secondary prevention after transient ischemic attack (TIA) or non-disabling stroke and prevention of thromboembolic events in heart failure with sinus rhythm;
clot formation in left atrium and thromboembolism in subjects undergoing cardioversion for cardiac arrhythmia; thrombosis before, during and after ablation procedure for cardiac arrhythmia;
venous thrombosis, this includes but not exclusively, treatment and secondary prevention of deep or superficial veins thrombosis in the lower members or upper member, thrombosis in the abdominal and thoracic veins, sinus thrombosis and thrombosis of jugular veins;
thrombosis on any artificial surface in the veins like catheter or pacemaker wires;
pulmonary embolism in patients with or without venous thrombosis;
Chronic Thromboembolic Pulmonary Hypertension (CTEPH);
arterial thrombosis on ruptured atherosclerotic plaque, thrombosis on intra-arterial prosthesis or catheter and thrombosis in apparently normal arteries, this includes but not exclusively acute coronary syndromes, ST elevation myocardial infarction, non ST elevation myocardial infarction, unstable angina, stent thrombosis, thrombosis of any artificial surface in the arterial system and thrombosis of pulmonary arteries in subjects with or without pulmonary hypertension;
thrombosis and thromboembolism in patients undergoing percutaneous coronary interventions (PCI);
cardioembolic and cryptogenic strokes;
thrombosis in patients with invasive and non-invasive cancer malignancies;
thrombosis over an indwelling catheter;
thrombosis and thromboembolism in severely ill patients;
cardiac thrombosis and thromboembolism, this includes but not exclusively cardiac thrombosis after myocardial infarction, cardiac thrombosis related to condition such as cardiac aneurysm, myocardial fibrosis, cardiac enlargement and insufficiency, myocarditis and artificial surface in the heart;
thromboembolism in patients with valvular heart disease with or without atrial fibrillation;
thromboembolism over valvular mechanic or biologic prostheses;
injuries or trauma in patients who had native or artificial cardiac patches, arterial or venous conduit tubes after heart repair of simple or complex cardiac malformations;
venous thrombosis and thromboembolism after knee replacement surgery, hip replacement surgery, and orthopedic surgery, thoracic or abdominal surgery;
arterial or venous thrombosis after neurosurgery including intracranial and spinal cord interventions;
congenital or acquired thrombophilia including but not exclusively factor V Leiden, prothrombin mutation, antithrombin III, protein C and protein S deficiencies, factor XIII mutation, familial dysfibrinogenemia, congenital deficiency of plasminogen, increased levels of factor XI, sickle cell disease, antiphospholipid syndrome, autoimmune disease, chronic bowel disease, nephrotic syndrome, hemolytic uremia, myeloproliferative disease, disseminated intra vascular coagulation, paroxysmal nocturnal hemoglobinuria and heparin induced thrombopenia;
thrombosis and thromboembolism in chronic kidney disease;
thrombosis and thromboembolism in end stage renal disease (ESRD);
thrombosis and thromboembolism in patients with chronic kidney disease or ESRD undergoing hemodialysis; and
thrombosis and thromboembolism in patients undergoing hemodialysis and/or extracorporeal membrane oxygenation.

In a specific aspect, a reversal agent according to the invention is for use in methods for neutralizing the therapeutic activity of an anti-FXIa antibody, and for use in related methods as essential part of a general bleeding management, in a patient being treated or administered the anti-FXIa antibody 076D-M007-H04-CDRL3-N110D to reduce the risk of stroke and/or systemic embolism, wherein the patient has ESRD and is undergoing dialysis.

In a specific aspect, a reversal agent according to the invention is for use in methods for neutralizing the therapeutic activity of an anti-FXIa antibody, and for use in related methods as essential part of a general bleeding management, in a patient being treated or administered the anti-FXIa antibody 076D-M007-H04-CDRL3-N110D to reduce the risk of stroke and/or systemic embolism, wherein the patient has non-valvular atrial fibrillation and ESRD and is undergoing dialysis.

In specific aspects, a subject, who may be in need of, or benefit from, the methods described herein (e.g., methods for neutralizing the therapeutic activity of an anti-FXIa antibody with anti-FXIa antibody reversal agents), has been treated with an anti-FXIa antibody (e.g., anti-FXIa antibody 076D-M007-H04-CDRL3-N110D) in combination with other agents for the prevention, treatment, or improvement of thromboembolic disorders. For example, statin therapies may be used in combination with FXIa antibodies and antigen binding fragments for the treatment of patients with thrombotic and/or thromboembolic disorders. Such subjects undergoing combination therapy may be in need of, or benefit from, the methods described herein (e.g., methods for neutralizing the therapeutic activity with anti-FXIa antibody reversal agents).

In a specific aspect, provided herein are methods for neutralizing the therapeutic activity of an anti-FXIa antibody, and related methods as essential part of a general bleeding management, in a patient being treated or administered an anti-FXIa antibody (e.g., anti-FXIa antibody 076D-M007-H04-CDRL3-N110D), said method comprises administering a reversal agent which specifically binds to the anti-FXIa antibody anti-FXIa antibody 076D-M007-H04-CDRL3-N110D, and neutralizes the therapeutic activity of the anti-FXIa antibody. In particular aspects, the bleeding or bleeding risk is associated with trauma, surgery, or post-delivery. In another particular aspect, the bleeding or bleeding risk is associated with emergency surgery or urgent procedures. In other particular aspects, the bleeding is life-threatening or uncontrolled. In specific aspects, the reversal agent is an antibody which specifically binds to anti-FXIa antibody 076D-M007-H04-CDRL3-N110D. In further specific aspects, the reversal agent is a Fab fragment of an antibody which specifically binds to anti-FXIa antibody 076D-M007-H04-CDRL3-N110D. In particular aspects, the reversal agent is an antibody or antigen-binding fragment thereof comprising amino acid sequences selected from Table 1. In particular aspects, the reversal agent is an antibody or antigen-binding fragment thereof, such as a Fab fragment, described herein, e.g., in Table 1, for example, antibodies “TPP-8236”, “TPP-8237”, “TPP-8238”, “TPP-8239”, “TPP-8240”, “TPP-8241”, “TPP-8343”, “TPP-8246”, “TPP-9238”, “TPP-9251”, “TPP-9252”, “TPP-9258”, monovalent antibody “TPP-20816” or Fab fragment “TPP-10089”; antibodies comprising VH CDRs and VL CDRs of such antibodies; antibodies that bind the same epitope(s) within target antibody anti-FXIa antibody 076D-M007-H04-CDRL3-N110D as such antibodies. In particular aspects, the reversal agent is an antibody or antigen-binding fragment thereof, such as a Fab fragment, comprising VH and VL amino acid sequences of antibody “TPP-9238”, “TPP-9251”, “TPP-9252” or “TPP-9258”, as set forth in Table 1. In particular preferred aspects, the reversal agent is an antibody comprising VH and VL amino acid sequences of antibody “TPP-9252”, as set forth in Table 1. In further particularly preferred aspects, the reversal agent is the corresponding Fab fragment “TPP-10089” of full-length IgG “TPP-9252”. In further particularly preferred aspects, the reversal agent is the corresponding monovalent antibody “TPP-20816” derived from full-length IgG “TPP-9252”.

In certain aspects, a temporary neutralization of the therapeutic activity of an anti-FXIa antibody (e.g., antibody 076D-M007-H04-CDRL3-N110D) is desired. In a particular aspect, provided herein are methods for neutralizing the therapeutic activity of an anti-FXIa antibody, and related methods as essential part of a general bleeding management in a patient treated or administered an anti-FXIa antibody such as antibody 076D-M007-H04-CDRL3-N110D, comprising the step of administering to the patient in need thereof, a reversal agent described herein, such as antibody “TPP-8236”, “TPP-8237”, “TPP-8238”, “TPP-8239”, “TPP-8240”, “TPP-8241”, “TPP-8343”, “TPP-8246”, “TPP-9238”, “TPP-9251”, “TPP-9252”, “TPP-9258”, monovalent antibody “TPP-20816”, or a Fab fragment thereof (e.g. Fab fragment “TPP-10089), once or twice, over a period of time (e.g., 1 hour to 24 hours or to 48 hours), followed by administering the anti-FXIa antibody, wherein a temporary neutralization of the therapeutic activity of the anti-FXIa antibody is achieved.

In certain aspects, an anti-FXIa antibody reversal agent described herein can be administered in combination with another anticoagulant reversal therapy. Non-limiting examples of conventional strategies for reversing anticoagulant effects include (i) fluid replacement using colloids, crystalloids, human plasma or plasma proteins such as albumin; or (ii) transfusion with packed red blood or whole blood. Examples of therapies for reversal of the effects of anticoagulants, for example, in cases of severe emergency, include, but are not limited to, prohemostasis blood components such as fresh frozen plasma (FFP), prothrombin complex concentrates (PCC) and activated PCC [(APCC); e.g. factor VIII inhibitor bypass activity (FEIBA)] and recombinant activated factor VII (rFVIIa).

In specific aspects, the present disclosure relates to methods for neutralizing the therapeutic activity of an anti-FXIa antibody (e.g., antibody 076D-M007-H04-CDRL3-N110D) in a patient being treated with the anti-FXIa antibody or antigen-binding fragment thereof, comprising (i) administering to the patient an effective amount of a reversal agent provided herein, e.g., a reversal agent (e.g., full-length antibody, monovalent antibody or antigen-binding fragment thereof, such as a Fab fragment) which binds an anti-FXIa antibody and is capable of neutralizing its therapeutic activity; and (ii) administering to the patient another anticoagulant reversal therapy, such as fresh frozen plasma (FFP), prothrombin complex concentrates (PCC), activated PCC or recombinant activated factor VII (rFVIIa). In specific aspects, the present disclosure relates to methods for neutralizing the therapeutic activity of an anti-FXIa antibody (e.g., antibody 076D-M007-H04-CDRL3-N110D) in a patient being treated with the anti-FXIa antibody or antigen-binding fragment thereof, comprising (i) administering to the patient an effective amount of a reversal agent provided herein, e.g., a reversal agent (e.g., full-length antibody, monovalent antibody or antigen-binding fragment thereof, such as a Fab fragment) which binds an anti-FXIa antibody and is capable of neutralizing its therapeutic activity; and (ii) administering to the patient fresh frozen plasma (FFP). In specific aspects, such method achieves homeostasis.

In certain aspects, provided herein is a method of managing bleeding in a patient being treated with an anti-FXIa antibody (e.g., antibody 076D-M007-H04-CDRL3-N110D), said method comprises temporarily reversing of the anticoagulant effect for a sufficient time to manage the bleeding. In specific embodiments, the step of reversing of the anticoagulant effect comprises (i) fluid replacement using colloids, crystalloids, human plasma or plasma proteins such as albumin; or (ii) transfusion with packed red blood or whole blood. In specific aspects, therapeutic agents for reversal of the effect of anticoagulants, for example, in cases of severe emergency, include, but are not limited to, prohemostasis blood components such as fresh frozen plasma (FFP), prothrombin complex concentrates (PCC) and activated PCC (APCC) (e.g. factor VIII inhibitor bypass activity (FEIBA)), and recombinant activated factor VII (rFVIIa).

In specific aspects, the present disclosure relates to methods for neutralizing the therapeutic activity of an anti-FXIa antibody (e.g., antibody 076D-M007-H04-CDRL3-N110D) in a patient being treated with the anti-FXIa antibody or antigen-binding fragment thereof, comprising (i) administering to the patient an effective amount of a reversal agent provided herein, which binds an anti-FXIa antibody and is capable of neutralizing its therapeutic activity; and (ii) administering to the patient another anticoagulant reversal therapy, such as rFVIIa (recombinant Factor Vla), emicizumab (ACE910), tranexamic acid, Fresh Frozen Plasma (FFP), Hemoeleven, Prothrombin Complex Concentrate (PCC), Activated PCC, or FEIBA (a FVIII inhibitor complex).

In certain aspects, in cases wherein administration of the reversal agents provided herein to a patient is not possible or not desired, the reversal agents according to this invention can also be used for extracorporeal depletion of an anti-FXIa antibody (e.g., antibody 076D-M007-H04-CDRL3-N110D). In specific aspects, extracorporeal depletion of an anti-FXIa antibody can for example be done by apheresis or dialysis. Therefore, a reversal agent according to this invention is immobilized onto a solid supporting surface. In preferred aspects of this invention, a full-length monoclonal antibody described herein, such as antibody “TPP-8236”, “TPP-8237”, “TPP-8238”, “TPP-8239”, “TPP-8240”, “TPP-8241”, “TPP-8343”, “TPP-8246”, “TPP-9238”, “TPP-9251”, “TPP-9252” or “TPP-9258” is used for this purpose. In especially preferred aspects antibody “TPP-9252” is used. Solid supporting surfaces for use in this method can be in form of beads or other solid matrices filled into columns or filter systems. These beads, other solid matrices, or filters can be coated with moieties, which are able to bind a reversal agent according to this invention in a way that does not block the reversal agent's active site during the reversal agent-anti-FXIa antibody interaction. In certain aspects, these moieties can for example, but not limited to, be selected from bacterial proteins including Protein A, G, L, Z, as well as recombinant derivatives thereof, linear, branched or cyclic peptides that bind specifically to the Fc-domain of antibodies, extracellular domains of Fc receptors or derivatives thereof, molecules like Streptavidin for capturing biotinylated antibodies, or chemical linker molecules with which the reversal agent is covalently linked to beads or other type of matrices.

In specific aspects, the risk of thromboembolic events including stroke, systemic embolism, coronary or peripheral artery thrombosis, venous thrombosis and pulmonary embolism increases with presence of predisposing factors such as thrombophilia, vessel wall damage and stasis. Evaluation of medical history, familiar antecedents and associated comorbidities can help to stratify patients according to their thromboembolic risks. In patients with atrial fibrillation, several scoring systems e.g., CHADS2 and CHA2DS2-VASc have been developed to assess stroke risk. Each was developed based on data from randomized trials, and clinical and epidemiologic cohort studies, and translated a weighted, multivariate formula of stroke risk factors to a simplified, easy-to-use mnemonic device, algorithm, calculator, or online tool. The CHADS2 risk score was used stratification tool to predict thromboembolic risk in atrial fibrillation patients (Lip (2011) Am J Med; 124(2): 111-4; Camm et al (2012) Eur Heart J; 33: 2719-2747); however, accumulated evidence shows that CHA2DS2-VASc is at least as good as or possibly better than, scores such as CHADS2 in identifying patients who develop stroke and thromboembolism and definitively better at identifying ‘truly low-risk’ patients with atrial fibrillation. The CHA2DS2-VASc score is presently recommended by Guidelines (Camm et al (2012) Eur Heart J 33, 2719-2747; January et al, AHA/ACC/HRS Atrial Fibrillation Guideline; J Am Coll Cardiol 2014; 64:2246-80) to guide the decision with regard to patients who should benefit of anticoagulant therapy and also to identify low risk patients in whom anticoagulation therapy is not warranted.

In certain aspects, subjects with a bleeding risk, for example a demonstrated high risk of bleeding, may be identified by previous medical history of bleeding, for example, bleeding during or after surgery or bleeding when treated with an anticoagulant (e.g. Warfarin). In addition, subjects with a bleeding risk, for example a demonstrated high risk of bleeding, may be identified by in vitro/ex vivo assays known in the art, for example, assays with a subject's plasma measuring aPTT and other biomarkers of the extrinsic coagulation pathways, such as prothrombin time (PT) and thrombin time (TT).

In certain aspects, methods for neutralizing the therapeutic activity of anti-FXIa antibody 076D-M007-H04-CDRL3-N110D with an anti-FXIa antibody reversal agent described herein, result in (i) reduction or reversal of the function blocking activity of the anti-FXIa antibody as determined by biochemical FXIa assays; (ii) reduction or reversal of the function blocking activity of the anti-FXIa antibody as determined by plasma based FXIa activity assays, (iii) reduction or reversal in aPTT prolongation as determined by plasma based aPTT assays and/or (iv) reduction or reversal of the anti-thrombotic activity of the anti-FXIa antibody as determined in plasma based aPPT assays in rabbits. In specific aspects, neutralization of the therapeutic activity is less than 100%, but is sufficient to achieve a clinically beneficial outcome. In further specific aspects, neutralization of the therapeutic activity is transient.

In certain aspects, methods for neutralizing the therapeutic activity of an anti-FXIa antibody 076D-M007-H04-CDRL3-N110D with an anti-FXIa antibody reversal agent described herein, result in reduction or reversal in aPTT prolongation, by at least 10%, at least 20%, at least 30%, at least 40%, at least 50%, at least 60%, at least 70%, at least 80%, or at least 90%.

### EXAMPLES

### Example 1: Generation of Anti-076D-M007-H04-CDRL3-N110D Antibodies

A fully human antibody phage display library (BioInvent n-CoDeR Fab lambda library) was used to isolate human monoclonal antibodies of the present invention by selection against solid phase immobilized antigen.

MaxiSorp™ Nunc-Immuno™ Tubes (immunotubes) (VWR, CatNo. 443990) were coated over night at 4° C. with the antigen (anti-FXIa antibody 076D-M007-H04-CDRL3-N110D/) (1 tube) and the off-target human Kallikrein (3 tubes), respectively. Immunotubes were washed and subsequently blocked for 1 h at room temperature (RT).

For depletion of off-target binders the blocked Fab-phage library was added to the blocked off-target loaded immunotubes and incubated for 10 min at room temperature with end-over-end rotation. This depletion step was repeated 3 times.

The depleted Fab-phage library was added to the blocked target loaded immunotube and incubated for 60 min at RT with end-over-end rotation.

After stringent washing (3× in blocking buffer and 9× in PBS (150 mM NaCl; 8 mM Na2HPO4; 1.5 mM KH2PO4; adjusted to pH=7.4-7.6) with 0.05% Tween-20) Fab-phages binding specifically to the coated target were eluted from the immunotubes by using trypsin solution (1 mg/ml, diluted in PBS). After a 30 min incubation step at RT eluted phages were transferred to a fresh tube. Aprotinin (2 mg/ml) was added to inhibit trypsin activity. The eluted phage stock was amplified in Escherichia coli strain HB101.

In the following selection rounds the target concentration was decreased in three doss steps of 500 nM to 200 nM to 100 nM to augment the selection pressure for high affinity binders.

For a first qualitative assessment, for each clone pool monoclonal cultivation and expression of 88 randomly picked Fab-phage clones was performed and subsequently tested for binding to the respective target used before for panning. A “binder” has been defined as a Fab-phage molecule showing in the ELISA assay at least a signal intensity of the average signal intensity of non-binding control Fab-phage molecules plus 10 times the standard deviation (average+10× standard deviation of non-target binding Fab-phage). From an overall number of 11960 tested Fab variants 55 binders with non-redundant sequences were selected as candidates (Example 4).

### Example 2: IgG and Fab Reformatting

Cloning of full human IgG₁ and Fab molecules for expression in eukaryotic expression systems.

The respective nucleic acid sequences encoding variable regions of the heavy and/or light chains of the reversal reagent antibody candidates were operatively linked, (such that the amino acid sequences encoded by the two DNA fragments are in-frame) to an antibody constant region by using recombinant DNA techniques (Sambrook, J. et al. eds., MOLECULAR CLONING: A LABORATORY MANUAL (2d Ed. 1989) Cold Spring Harbor Laboratory Press, NY. Vols. 1-3). The sequences of human heavy chain and light chain constant regions are known in the art (see e.g., Kabat, E. A., el al. (1991).

As a first step for the generation of Fabs and of human IgG₁ DNA fragments encoding VH, VL, human lambda constant, and CH1 constant domain of human IgG₁ Fc were synthesized.

For the generation of Fabs the VH fragment and the human IgG₁ CH1 domain fragment were assembled into one pTT5 expression plasmid, whereas the VL fragment and the human lambda constant IgG₁ domain fragment were assembled in a second pTT5 expression plasmid. The co-expression of both plasmids in HEK293E cells resulted in the molecules of interest.

For the generation of full-length human IgG₁ antibodies, the VH fragment and the human IgG₁ Fc domain fragment were assembled into one pTT5 expression plasmid, whereas the VL fragment and the human lambda constant IgG₁ domain fragment were assembled in a second pTT5 expression plasmid. The co-expression of both plasmids in HEK293E cells resulted in human IgG₁ molecule.

### Example 3: Expression and Quantification of Antibodies and Antibody Variants

The above mentioned IgGs were transiently expressed in mammalian cells as described in Tom et al., Chapter 12 in Methods Express: Expression Systems edited by Micheal R. Dyson and Yves Durocher, Scion Publishing Ltd, 2007. Briefly, a CMV-Promoter based expression plasmid is was transfected into HEK293-6E cells and incubated in Fernbach-Flasks or Wave-Bags. Transfected cells were cultivated at 37° C. for 5 to 6 days in F17 Medium (Invitrogen). 1% Ultra-Low IgG FCS (Invitrogen) and 0.5 mM valproic acid (Sigma) was supplemented 24 h post transfection.

IgGs were separated from cells by centrifugation. The IgG concentration was assessed by an IgG-Fc quantification ELISA according to well-known methods in the art. Briefly, 1:1500 diluted supernatant and a 2-fold dilution series of Human Reference Serum (Bethyl, RS-110-4) starting with 400 ng/ml were immobilized in black Maxisorp 384 micro titer plates (MTP) coated with anti-human Fc [Sigma 12136] in a 1:440 dilution in 1× coating buffer (Candor, 121125) for 1 h, 37° C. After blocking with 100% SMART Block (Candor, 113125) anti-human Fc-HRP [Sigma, A0170] was applied in a 1:10000 dilution for the detection of antibodies in supernatants of transfected cells and in reference samples.

Antibodies were purified by Protein A chromatography and further characterized by their binding affinity using an Enzyme-linked immunosorbent assay (ELISA).

Fabs were purified from sterile filtered HEK293 6E supernatants using a 3-step research downstream process. As capture step a “Capture Select IgG-CH1” affinity column (Life Technologies) equilibrated in PBS pH 7.4 was used. After washing in wash buffer (PBS pH 7.4) for 10 column volumes, elution of the Fab was achieved using Glycine 0.1M pH 3.0 (6 CV). Upon neutralization with Tris Base a size exclusion chromatography (Superdex 200 50/60 increase GL, GE Healthcare) was used for buffer exchange into DPBS pH 7.4 and aggregate removal. Analytical size exclusion chromatography demonstrated that no dimer was present in the resulting batch.

For quantification of full-length antibodies, the anti-human IgG Fc specific antibody (I2136, Sigma) was coated at a concentration of 5 μg/ml over night at 4° C. to 384-well microtiter plates (Nunc). Solutions containing the IgGs of interest were added at different concentrations an incubated for 1 hour at room temperature. For detection, the detection antibody AG170 (Sigma) and as substrate Amplex Red were added. Fluorescence was monitored at 535/590 nm using a SpectraFluorplus Reader (Tecan).

For quantification of antibody variants like Fabs, the Human Kappa ELISA Kit (Abcam, ab157709) was used according to the manufacturer's instructions.

### Example 4: Enzyme-Linked Immunosorbent Assay (ELISA)

A standard ELISA format was used for analyzing the binding affinity of the reversal agents of this invention to 076D-M007-H04-CDRL3-N110D. This antigen was coated to black 384 well Maxisorp microtiter plates (Nunc; Cat. No: 460518), diluted to a concentration of 1 μg/ml in 1× Coating Buffer (Candor Bioscience; Cat. No. 121125). Plates were incubated overnight at 4° C. After overnight incubation, plates were washed 2× with 50 μl/well using PBS+0.05% Tween 20. Following this, 50 μl/well of blocking buffer (Smart Block; Candor Bioscience; Cat. No. 113500) was added and the plates were incubated for 1 hour at room temperature. Afterwards, plates were washed for 3× using 50 μl/well of a PBS+0.05% Tween 20 buffer. Antibodies of this invention were added at different concentrations in a final volume of 30 μl/well. Plates were incubated for 1 hour at room temperature. Following this incubation step, plates were washed for 3× using 50 μl/well of a PBS+0.05% Tween 20 buffer. For the detection of bound reversal agents, the anti-Human Lambda Light Chains (Bound and Free)—Peroxidase antibody (Sigma; Cat. No. A5175) was diluted by the factor of 1:10.000 in 10% Blocking Buffer. 30 μl/well of this diluted detection antibody was added and plates are incubated for 1 hour at room temperature. Following this incubation step, plates were washed for 3× using 50 μl/well of a PBS+0.05% Tween 20 buffer. As substrate, a mixture of 30 μl/well of 1:1000 diluted Amplex red (Invitrogen; Cat. No. 12222; stock solution 10 mM in DMSO) and 1:10.000 of Hydrogen peroxide (Merck; Cat. No. 107209; 30% stock solution) was added and the plates incubated for 20 minutes in the dark.

For measurement, the Infinite f500 reader (Tecan) was used. Measurement mode: Fluorescence; Top reading; Ex 535 nm; Em 590 nm.

Data were analyzed using the GraphPadPrism software. The binding activities of the Reversal Agents of this invention were calculated as EC50 values. Two to three independent experiments were performed in quadruplicate.

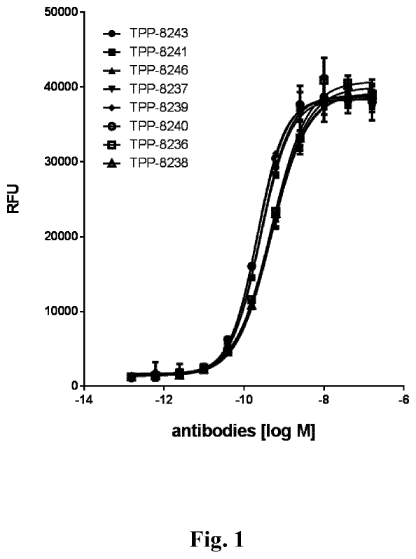

From an overall number of 11960 tested Fab variants 55 binders with non-redundant sequences were selected as candidates. Whereas for the majority of these 55 binders binding activities were in the lower three-digit nanomolar range. the following antibodies showed the most effective binding activity: TPP-8243 (SEQ ID NO 95 and SEQ ID NO. 96) TPP-8241 (SEQ ID NO 81 and SEQ ID NO. 82), TPP-8246 (SEQ ID NO. 109 and SEQ ID NO. 110), TPP-8237 (SEQ ID NO. 25 and SEQ ID NO. 26), TPP-8239 (SEQ ID NO. 53 and SEQ ID NO. 54), TPP-8240 (SEQ ID NO. 67 and SEQ ID NO. 68), TPP-8236 (SEQ ID NO. 11 and SEQ ID NO. 12), and TPP-8238 (SEQ ID 39 and SEQ ID 40) (FIG. 1). The corresponding EC50 values are listed in Table 2.

TABLE 2
Summary of binding data for the most effective binders identified:

| TPP No. | EC50 [log M] |
|---|---|
| TPP-8243 | 2.56E−10 |
| TPP-8241 | 2.592E−10 |
| TPP-8246 | 4.775E−10 |
| TPP-8237 | 4.902E−10 |
| TPP-8239 | 2.119E−10 |
| TPP-8240 | 2.158E−10 |
| TPP-8236 | 4.845E−10 |
| TPP-8238 | 4.847E−10 |

As shown in Table 2, for these candidates, binding activities to 076D-M007-H04-CDRL3-N110Din the sub-nanomolar range have been determined.

### Example 5: Activity testing

In order to determine the function blocking activity of the potential reversal agents, the catalytic activity of human FXIa was determined. For this, the activity of FXIa (Haematologic Technologies, Inc., catalogue number HCXIA-0160) was determined by measuring the cleavage of a specific, fluorogenically-labeled substrate (I-1575, Bachem, final concentration 25 μM) and the fluorescence was monitored continuously at 360/465 nm using a SpectraFluorplus Reader (Tecan Infinite M1000Pro).

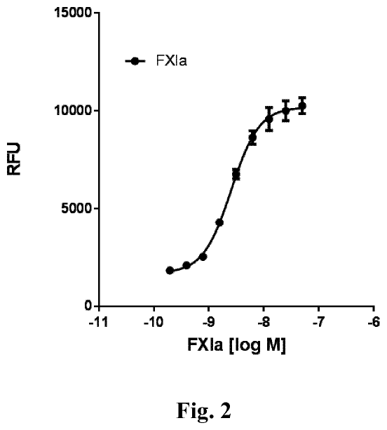

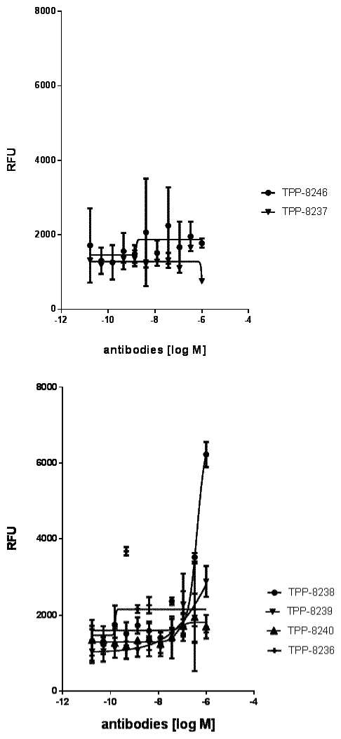

For testing the FXIa blocking activity of the anti-FXIa antibody 076D-M007-H04-CDRL3-N110D, a range of concentrations (50-25-12.5-6.25-3.125-1.56-0.78-0.39-0.19 nM) of this antibody was pre-incubated for 10 minutes at 37° C. with 10 nM FXIa in a buffer containing 50 mM Tris/HCl, 100 mM NaCl, 5 mM CaCl₂) and 0.1% BSA. Following this incubation step, the substrate I-1575 was added, the signals from the plates were measured and the data analyzed. As shown in FIG. 2, EC50 values of human FXIa were 1 to 2 nanomolar.

For testing the neutralizing activity of the potential reversal agents, these antibodies were pre-incubated in dose-effect concentrations starting with 160 nM, followed by 1:4 dilutions for 10 dilution steps for 10 minutes at 37° C. with 1 nM of anti-FXIa antibody 076D-M007-H04-CDRL3-N110D. Following this incubation step, 10 nM FXIa in a buffer containing 50 mM Tris/HCl, 100 mM NaCl, 5 mM CaCl₂) and 0.1% BSA was added. This mixture was incubated for 10 minutes at 37° C. Following this incubation step, the substrate I-1575 was added, the signals from the plates were measured and the data were analyzed.

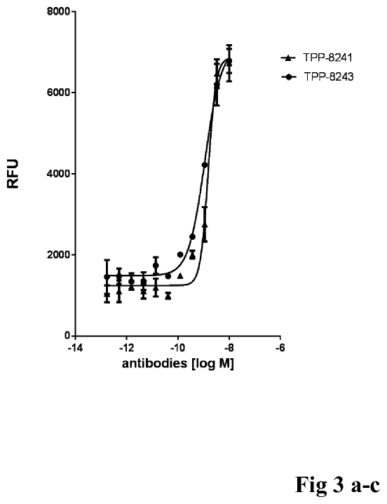

The neutralizing activities of the antibodies of this invention are shown in FIGS. 3 a-c and are listed in Table 3. (IC50 values are given in nanomolar).

TABLE 3
Neutralizing activity of selected antibodies of
this invention expressed in IC50 as log M values:

| TPP | IC50 [M] |
|---|---|
| TPP-8246 | >1.00E−06 |
| TPP-8237 | >1.00E−06 |
| TPP-8238 | 75% inhibition at 1.00E−06 |
| TPP-8239 | >1.00E−06 |
| TPP-8240 | >1.00E−06 |
| TPP-8236 | >1.00E−06 |
| TPP-8243 | 1.126E−09 |
| TPP-8241 | 1.522E−09 |

Only two of the eight antibodies showing high binding activity to the antigen 076D-M007-H04-CDRL3-N110D namely TPP8243 and TPP-8241, were able to neutralize the function blocking activity of anti-FXIa antibody 076D-M007-H04-CDRL3-N110D significantly and in a dose dependent manner.

### Example 6: Plasma Based Activity Assay

In order to analyze neutralizing activity of TPP-8241 and TPP-8243 in more depth, a plasma based FXIa assay was used. For this human citrate buffered plasma (Harlan Laboratories) was diluted in a buffer composed of 50 mM Tris/HCl, 100 mM NaCl, pH 7.4 to a final concentration of 30%. To avoid unspecific cleavage of the FIXa substrate 299F (American Diagnostica) a specific Thrombin inhibitor was added at a final concentration of 1 μM. Additionally, phospholipids at a concentration of 9% were added. For testing the neutralizing activity of the two antibodies, these were diluted at various concentrations in the plasma/buffer mixture. In a next step, anti-FXIa antibody 076D-M007-H04-CDRL3-N110D was added at a fixed concentration of 1 nM. These mixtures were incubated for 30 minutes at room temperature. To induce the intrinsic coagulation pathway, the insoluble aluminum silicate Kaolin and CaCl₂) were added at final concentrations of 12 μg/ml and 12 mM, respectively. For detecting FIXa activity, generated via the conversion of the corresponding zymogen FIX by FXIa, the flurogenic Thrombin substrate 299F (American Diagnostica) was added at a final concentration of 140 μM and the fluorescence was monitored continuously at 360/465 nm using a SpectraFluorplus Reader (Tecan). Afterwards, the data were analyzed using the GraphPadPrism software.

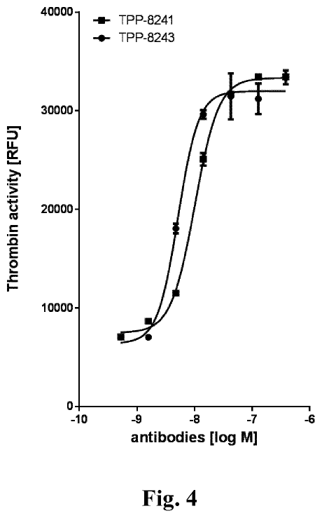

As shown in FIG. 4, in this assay, TPP-8241 and TPP-8243 exhibited IC50 values of 10 nM and 5 nM, respectively.

### Example 7: Activated Partial Thromboplastin Time (aPTT)

Aliquots of plasma were incubated with increasing concentrations of the antibodies of this invention for 3 min at 37° C. To initiate the intrinsic coagulation pathway, 0.05 ml of plasma was incubated with 0.05 ml of aPTT reagent (Diagnostica Stago, K.C Prest 5) for exactly 3 min. Coagulation was started by re-calcifying the samples with 0.05 ml of 0.025 M pre-warmed calcium chloride solution. An automated coagulometer (AMAX 200, Trinity Biotech, Lemgo, Germany) mixed the plasma at 37° C. and mechanically recorded the time to clotting. The test drug concentration prolonging aPTT by a factor of 1.5 is calculated and reported as EC150 or 1.5 times of elongation. Results are listed in Table 4.

TABLE 4
aPTT values (EC150, μM) for the antibodies of this invention.

| designation | aPTT 1.5 X [μM] |
|---|---|
| TPP-8241 | 0.33 |
| TPP-8243 | 0.30 |

### Example 8: Germlining and Sequence Optimization of Reversal Agents

Due to its better activity in the plasma-based activity assay, TPP-8243 was chosen for further optimization. In order to reduce the intrinsic immunogenicity risk, those molecules showing the most promising activity regarding the neutralization of selected reversal agents were selected for further sequence optimization and germlining.

Therefore, amino acids which differ from the nearest germline sequence were exchanged, the corresponding cDNAs were synthesized, HEK293 cells were transiently transfected, the expressed antibodies of this invention were quantified and tested for their ability to bind anti-FXIa antibody 076D-M007-H04-CDRL3-N110D.

Outcome of this approach were four antibodies TPP-9251 (SEQ ID NO. 137 and SEQ ID NO. 138), TPP-9252 (SEQ ID NO. 151 and SEQ ID NO. 152), TPP-9258 (SEQ ID NO. 165 and SEQ ID NO. 166) and TPP-9238 (SEQ ID NO. 123 and SEQ ID NO. 124).

### Example 9: Comparative Activity Analysis of Antibodies of this Invention

In direct comparison to TPP-8243 (SEQ ID NO 95 and SEQ ID NO. 96), TPP-9251 (SEQ ID NO. 137 and SEQ ID NO. 138), TPP-9252 (SEQ ID NO. 151 and SEQ ID NO. 152), TPP-9258 (SEQ ID NO. 165 and SEQ ID NO. 166) and TPP-9238 (SEQ ID NO. 123 and SEQ ID NO. 124) were tested for their neutralizing activity in the biochemical FXIa assay (as described in Example 5), in the plasma based assay (as described in Example 6) as well as in the aPTT assay (as described in Example 7). Results are shown in Table 5:

TABLE 5
Comparative activity analysis of different
antibodies of this invention

| | biochemical FXIa assay | plasma based assay | aPTT 1.5 |
|---|---|---|---|
| designation | IC 50 [nM] | IC 50 [nM] | X [μM] |
| TPP-8243 | 1.1 | 5 | 0.3 |
| TPP-9251 | 0.2 | 6 | 0.06 |
| TPP-9252 | 0.2 | 5 | 0.05 |
| TPP-9258 | 0.5 | 7 | 0.1 |
| TPP-9238 | 0.5 | 10 | 0.1 |

Surprisingly, for a 1.5-fold induction of the activated partial prothrombin time, a 6 fold lower concentration was necessary for TPP-9252 than for example for the initial variant TPP-8243.

### Example 10: Fab Generation and Testing

As alternative to the full-length IgG TPP-9252, the corresponding Fab fragment TPP-10089 was also produced and tested for its activity in vitro and in vivo.

For this, HEK 293 cells were transiently transfected a mammalian expression vector encoding for the Fab fragment. This molecule was purified from sterile filtered cell culture supernatants using a 3-step process. As capture step a “Capture Select IgG-CH1” affinity column (Life Technologies) equilibrated in PBS pH 7.4 was used. After washing in wash buffer (PBS pH 7.4) for 10 column volumes, elution of the Fab was achieved using Glycine 0.1M pH 3.0 (6 CV). Upon neutralization with Tris Base a size exclusion chromatography (Superdex 200 50/60 increase GL, GE Healthcare) was used for buffer exchange into DPBS pH 7.4 and aggregate removal. Analytical size exclusion chromatography demonstrated that no dimer was present in the resulting batch. Designation of this Fab molecule is TPP-10089.

The activity of TPP-10089 was tested as described in Example 4, Example 5, Example 6, and Example 7.

A comparison of the activity of the full-length IgG (TPP-9252) versus the corresponding Fab fragment (TPP-10089) and the monovalent antibody TPP-20816 in certain assay systems is given in Table 6.

TABLE 6
Activity of antibody TPP-9252, the corresponding Fab fragment
TPP-10089 and the monovalent antibody TPP-20816

| | TPP- | TPP- | TPP- |
|---|---|---|---|
| activity | 9252 | 10089 | 20816 |
| binding activity [EC50 nM] | 0.1 | 0.1 | |
| biochemical FXIa assay [IC50 nM] | 1.1 | 1 | 1 |
| (tested @ 1 nM 076D-M007-H04- | | | |
| CDRL3-N110D) | | | |
| plasma-based assay [IC50 nM] | 5 | 5 | |
| aPTT [EC150 μM] | 0.05 | 0.06 | 0.07 |
| (tested @ 0.1 μM 076D-M007-H04- | | | |
| CDRL3-N110D) | | | |

In most assay formats, the activities of TPP-9252, TPP-10089, and TPP-20816 are comparable. Especially in the plasma-based assay as well as in the aPTT assay, the activities of the full-length IgG compared to the corresponding Fab are barely distinguishable.

### Example 11: In Vivo Testing

To test if TPP-9252 can reverse the anti-thrombotic activity of anti-FXIa antibody 076D-M007-H04-CDRL3-N110D, a PD study was performed in rabbits (New Zealand White) in a short time model under anesthesia (Ketamine/Xylazine). A single dose of anti-FXIa antibody 076D-M007-H04-CDRL3-N110D (3 mg/kg) was administered to male rabbits followed by single applications of the reversal agent TPP-9252 (1.5-5-15 mg/kg). The 3 mg/kg of anti-FXIa antibody 076D-M007-H04-CDRL3-N110D were administered 15 min prior to the applications of TPP-9252.

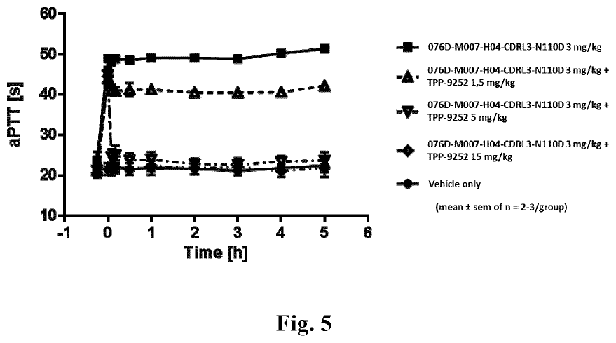

As shown in FIG. 5, aPTT was nearly at baseline level (>90% normalization) after 5 mg/kg TPP-9252, which corresponds to a molar excess of 1.7 fold. When anti-FXIa antibody 076D-M007-H04-CDRL3-N110D was given in excess (at 1.5 mg/kg TPP-9252) only a minor decrease in aPTT was observed. Outcome of this experiment was, that a molar excess of the reversal agent greater than 2 fold is expected to provide full return to baseline.

In order to analyze the effect of the corresponding Fab fragment TPP-10089 on aPTT normalization, this Fab fragment was administered at a concentration of 10 mg/kg 2 times with a time interval of 60 min.

In contrast to TPP-9252, which leads to a long-lasting normalization of the aPTT, the effect induced by TPP-10089 is only transient.

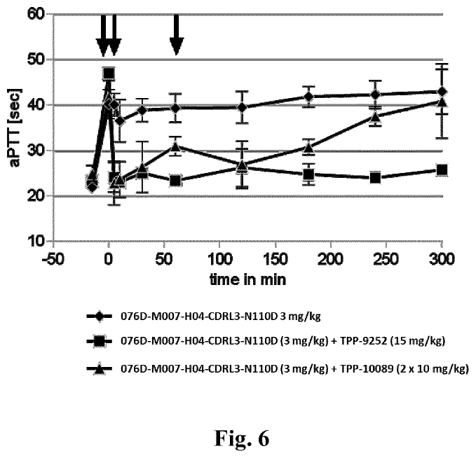

Following an initial drop to aPTT baseline after the first administration of TPP-10089, a slow but steady increase in aPTT elongation was observable. After a time period of 5 hours, aPTT elongation was at the same level as the treatment group which received the therapeutic antibody only. Even the second application of 10 mg/kg of TPP-10089 led only to a partial reduction of aPTT time (see FIG. 6).

### Example 12: Generation of a Monovalent Antibody and Testing Thereof

As alternative to the full-length IgG TPP-9252, a monovalent antibody TPP-20816 derived from TPP-9252 was also produced and tested for its activity in vitro. TPP-20816 was expressed as described in Example 2, with the following variation. For the generation of monovalent human IgG₁ antibodies, one VH fragment and the human IgG₁ Fc domain fragment were assembled into one pTT5 expression plasmid (SEQ ID NO: 191), the second human IgG₁ Fc domain fragment was assembled into another pTT5 expression plasmid (SEQ ID NO: 193), whereas the VL fragment and the human lambda constant IgG₁ domain fragment were assembled in a third pTT5 expression plasmid (SEQ ID NO: 192). The co-expression of all three plasmids in HEK293E cells resulted in the monovalent human IgG₁ molecule TPP-20816.

TPP-20816 was purified as described in Example 3.

The activity of the monovalent antibody was tested as described in Example 4 and Example 7.

A comparison of the activity of the full-length IgG (TPP-9252) versus the corresponding Fab fragment (TPP-10089) and the monovalent antibody TPP-20816 in certain assay systems is given in Table 6.

1: A monoclonal antibody or antigen-binding fragment thereof that specifically binds to anti-FXIa antibody 076D-M007-H04-CDRL3-N110D and thereby inhibits the neutralizing activity of antibody 076D-M007-H04-CDRL3-N110D, wherein the antibody or antigen binding fragment thereof comprises HCDR1-3 and LCDR1-3 comprising the amino acid sequences of:
a) SEQ ID NOs: 72, 73, 74, 76, 77, and 78, respectively;
b) SEQ ID NOs: 86, 87, 88, 90, 91, and 92, respectively;
c) SEQ ID NOs: 114, 115, 116, 118, 119, and 120, respectively;
d) SEQ ID NOs: 128, 129, 130, 132, 133, and 134, respectively;
e) SEQ ID NOs: 142, 143, 144, 146, 147, and 148, respectively;
f) SEQ ID NOs: 156, 157, 158, 160, 161, and 162, respectively;
g) SEQ ID NOs: 170, 171, 172, 174, 175, and 176, respectively; or
h) SEQ ID NOs: 184, 185, 186, 188, 189, and 190, respectively.

2: The monoclonal antibody or antigen-binding fragment according to claim 1, wherein the antibody or antigen binding fragment thereof comprises a heavy chain sequence and a light chain sequence comprising the amino acid sequences of:
a) SEQ ID NOs: 81 and 82, respectively;
b) SEQ ID NOs: 95 and 96, respectively;
c) SEQ ID NOs: 123 and 124, respectively;
d) SEQ ID NOs: 137 and 138, respectively;
e) SEQ ID NOs: 151 and 152, respectively;
f) SEQ ID NOs: 165 and 166, respectively;
g) SEQ ID NOs: 179 and 180, respectively; or
h) SEQ ID NOs: 191 and 192, respectively.

3: The monoclonal antibody or antigen-binding fragment thereof according to claim 1, wherein the antibody or antigen binding fragment thereof comprises HCDR1-3 and LCDR1-3 comprising the amino acid sequences of SEQ ID NOs: 142, 143, 144, 146, 147, and 148, respectively.

4: The monoclonal antibody according to claim 1, wherein the antibody or antigen binding fragment thereof comprises the heavy chain sequence of SEQ ID NOs: 151 and the light chain sequence of SEQ ID NO: 152.

5: The monoclonal antibody or antigen-binding fragment according to claim 1, wherein the antibody or antigen binding fragment thereof is chimeric, humanized, or human.

6: The monoclonal antibody according to claim 1, wherein the antibody comprises a human IgG heavy chain constant region.

7: The monovalent antibody or antigen-binding fragment thereof according to claim 1, wherein the antibody or antigen binding fragment thereof comprises HCDR1-3 and LCDR1-3 comprising the amino acid sequences of SEQ ID NOs: 184, 185, 186, 188, 189, and 190, respectively.

8: The monovalent antibody or antigen-binding fragment thereof according to claim 1, wherein the antibody or antigen finding fragment thereof comprises the heavy chain sequences of SEQ ID NOs: 191 and 193, respectively and the light chain sequence of SEQ ID NO: 192.

9: The antigen-binding fragment according to claim 1, wherein the antigen-binding fragment is a Fab fragment.

10: The antigen-binding fragment according to claim 9, wherein the antigen-binding fragment comprises HCDR1-3 and LCDR1-3 comprising the amino acid sequences of SEQ ID NOs: 170, 171, 172, 174, 175, and 176, respectively.

11: The antigen-binding fragment according to claim 9, wherein the antigen-binding fragment comprises the heavy chain sequence of SEQ ID NOs: 179 and the light chain sequence of SEQ ID NO: 180.

12: A polynucleotide encoding a monoclonal antibody or antigen-binding fragment thereof that specifically binds to anti-FXIa antibody 076D-M007-H04-CDRL3-N110D and thereby inhibits the neutralizing activity of antibody 076D-M007-H04-CDRL3-N110D, wherein the antibody or antigen binding fragment thereof comprises HCDR1-3 and LCDR1-3 comprising the amino acid sequences of:
a) SEQ ID NOs: 72, 73, 74, 76, 77, and 78, respectively;
b) SEQ ID NOs: 86, 87, 88, 90, 91, and 92, respectively;
c) SEQ ID NOs: 114, 115, 116, 118, 119, and 120, respectively;
d) SEQ ID NOs: 128, 129, 130, 132, 133, and 134, respectively;
e) SEQ ID NOs: 142, 143, 144, 146, 147, and 148, respectively;
f) SEQ ID NOs: 156, 157, 158, 160, 161, and 162, respectively;
g) SEQ ID NOs: 170, 171, 172, 174, 175, and 176, respectively: or SEQ ID NOs: 184, 185, 186, 188, 189, and 190, respectively.

13: A vector, which comprises a polynucleotide as defined in claim 12.

14: A host cell comprising a vector according to claim 13.

15: A process for the production of a monoclonal antibody or antigen-binding fragment thereof, said process comprising culturing a host cell defined in claim 14 under conditions allowing the expression of the monoclonal antibody or antigen-binding fragment thereof.

16: A pharmaceutical composition comprising a monoclonal antibody or antigen-binding fragment thereof according to claim 1 and a pharmaceutically acceptable excipient.

17. (canceled)

18: A method of neutralizing the therapeutic activity of anti-FXIa antibody 076D-M007-H04-CDRL3-N110D in a patient treated with anti-FXIa antibody 076D-M007-H04-CDRL3-N110D comprising administering to the patient an effective amount of the monoclonal antibody or antigen-binding fragment thereof according to claim 1.

19: A kit comprising the pharmaceutical composition according to claim 16.

20: A method of extracorporeal depletion of anti-FXIa antibody 076D-M007-H04-CDRL3-N110D in a patient treated with anti-FXIa antibody 076D-M007-H04-CDRL3-N110D comprising administering to the patient an effective amount of the monoclonal antibody or antigen-binding fragment thereof according to claim 1.

21: The process of claim 15, further comprising recovering the produced antibody or antigen-binding fragment thereof from the culture.
